(19) United States
(12) Patent Application Publication
(10) Pub. No.: US 2009/0069256 A1
(43) Pub. Date: Mar. 12, 2009

(54) Enhancing protein expression

(76) Inventors: Larry R. Smith, San Diego, CA (US); Vafa Shahabi, Valley Forge, PA (US); Maninder K. Sidhu, New City, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/628,455

(22) PCT Filed: Jun. 6, 2005
(86) PCT No.: PCT/US05/19592

§ 371 Date: Nov. 8, 2007

Related U.S. Application Data
(60) Provisional application No. 60/576,819, filed on Jun. 4, 2004.

(51) Int. Cl. A61K 31/7088; C12N 15/11; C12N 15/87; A61P 43/00; C12N 15/00

(52) U.S. Cl. 514/44; 536/23.1; 536/23.53; 536/23.5; 536/23.6; 536/23.7; 536/23.72; 536/23.74; 435/455; 435/320.1

(57) ABSTRACT

Modified polynucleotide compositions providing enhanced gene expression and methods for preparing said compositions are disclosed. Methods of using the compositions, such as in screening assays, diagnostic tools, kits, etc. and for prevention and/or treatment of diseases and disorders are also disclosed.

### FIELD OF THE INVENTION

The present invention relates to polynucleotide compositions that provide enhanced efficiency in the expression of proteins or polypeptides by genes in mammalian cells (i.e., resulting in an increase in the levels of the proteins or polypeptides encoded by the genes), such as viral, bacterial and mammalian genes, as well as methods for preparing said compositions. In particular, the invention provides polynucleotide sequences that provide enhanced gene expression over the corresponding wild-type polynucleotides. Also provided are methods of using the polynucleotide compositions in prevention and treatment of diseases and disorders (e.g., immuno-therapeutic, immuno-prophylactic and genetic therapy uses and the like), such as in DNA and RNA vaccines (e.g., DNA vaccines for preventing/treating HIV/AIDS) as well as in biological assays, diagnostics and the like.

### BACKGROUND OF THE INVENTION

The level of protein expressed by a gene is crucial to in vivo responses/effects involving the protein, as well as in vitro assays involving the protein. Under some circumstances and for reasons not fully characterized, however, in vitro and/or in vivo benefits of the protein product of a gene are compromised because the gene is not adequately expressed in cells. Poor protein expression is encountered in a number of different contexts. For example, poor expression of proteins by eukaryotic genes in prokaryotic cells has been previously reported (see Seed et al., U.S. Pat. Nos. 5,786,464 and 5,795,737). The poor expression of proteins by viral genes in mammalian cells has also been described (see Schwartz et al., J. Virol. 66(12):7176-7182 (1992), Schneider et al., J. Virol., 71(7):4892-4903 (1997) and Pavlakis et al., U.S. Pat. No. 6,414,132 B1). However, the poor expression of certain viral, bacterial and mammalian genes, in mammalian cells remains a significant problem from the standpoint of both in vivo uses of the protein products and in vitro uses in assays and the like.

There are a number of factors that influence the levels of gene expression of proteins in mammalian cells and that account for, or at least contribute to, the poor expression observed for certain genes in these cells. In some instances, translational mechanisms are responsible for the poor expression. For example, it has been recognized that in certain wild-type genes, the naturally occurring nucleic acid sequences of the genes are rich in adenine (A) and/or uracil (U) (if the polynucleotide is RNA) or adenine (A) and/or thymine (T) (if the polynucleotide is DNA) and biased toward “disfavored codons”. The term “disfavored codons,” as used herein, refers to codons that contain A, U, or T in the third (“wobble”) position of the codon nucleotide triplet. It has been suggested in the art (see Haas et al., Current Biol. 6:315-324, 1996) that certain wild-type genes are not handled efficiently by the translational machinery of mammalian cells.

Also, in addition to translational mechanisms accounting for poorly-expressed genes, there have been various AU rich RNA instability sequences discovered in several messenger RNAs (mRNAs) which do not directly impact the translatability of a given mRNA, but limit protein expression by increasing mRNA turnover. Further, several specific “inhibitory” sequences contained within the HIV-1 gag ORF have been described (see Pavlakis, U.S. Pat. No. 6,414,132 B1) which limit the expression levels of gag by inhibiting nuclear export of these transcripts.

IL-15 exemplifies the problem inherent in poor gene expression. IL-15 is a pluripotent cytokine that is secreted by antigen presenting cells such as monocytes/macrophages and dendritic cells, but also a variety of nonlymphoid tissues. IL-15, in addition to being a growth and survival factor for memory CD8+ T cells, is also a potent activator of effector-memory CD8+ T cells, both in healthy and HIV-infected individuals. Because IL-15 is a prototypic Th1 cytokine, and by virtue of its activity as a stimulator of T cells, NK cells, LAK (lymphokine-activated killer) and TILs (tumor infiltrating lymphocytes), IL-15 is a potential candidate for use as a molecular adjuvant along with HIV DNA vaccines to enhance cellular immune responses. However, one major limiting factor for its use as a genetic adjuvant, remains its poor expression due to its complex regulation at the levels of mRNA transcription and translation and, protein translocation and secretion.

Further, DNA vaccines, which are being studied for many diseases, including HIV, influenza, tuberculosis and malaria, usually work by injecting specially reproduced genetic material of the organism directly into the body. This genetic material encodes information that gets the individual's own cells to make the vaccine. DNA vaccines have shown some impressive results in animals. Studies by Merck & Co. demonstrated that a DNA vaccine can prevent influenza in animals.

In the area of HIV disease, DNA vaccines have generally not been able to stimulate strong immune responses in people. It has been suggested that DNA vaccines are less effective in humans than in smaller animals as a result of the problem of scaling up doses, where it is not practical to give large enough amounts of these vaccines to match the doses given to mice or monkeys. Interest in DNA vaccines either for prevention or treatment is therefore likely to depend on finding new and more efficient ways to present them to the immune system. An approach that improves the expression of a protein, such as IL-15 for use as an adjuvant in a DNA vaccine against HIV/AIDS, for example, is thus highly desirable.

Various techniques have been proposed for optimizing expression of genes, particularly for poorly expressed genes. For example, one approach involved selectively replacing wild-type codons encompassing inhibitory sequences with other codons to eliminate the inhibitory effect. However, the sequence motifs that define either instability or inhibitory sequences are not readily apparent and therefore not easily identified. Several genes (e.g. E7 and En among others) which appear to also contain inhibitory sequences have not yet been mapped to identify the location of inhibitory sequences and there are no straightforward prescriptions from the gag work to predict how to eliminate inhibitory sequences from these genes.

Further, a complete “codon optimized” version of gp120 envelope has been described (see Haas et al., Current Biology, 6:315-324, 1996; Andre et al., J. Virology, 72:1497-1503) in which all “non-preferred” wild-type codons from env were replaced with “preferred” codons and found to enhance expression levels.

Previously available approaches, as described above, impose stringent requirements in their application. In particular, these approaches require the use of “preferred codons,” or alternatively, identification of specific “inhibitory sequences.” For example, the technology described by Seed requires incorporation of “preferred codons” and purportedly depends on invoking the translational enhancement as the mechanism of increased protein levels.

“Preferred codons,” as defined by Seed, are GCC for Ala, CGC for Arg, AAC for Asn, GAC for Asp, TGC for Cys, CAG for Gln, GGC for Gly, CAC for His, ATC for lie, CTG for Leu, AAG for Lys, CCC for Pro, TTC for Phe, AGC for Ser, ACC for Thr, TAC for Tyr, and GTG for Val. According to Seed, “less preferred codons” are GGG for Gly, ATT for lie, CTC for Leu, TCC for Ser, and GTC for Val. Seed also teaches that all codons which do not fit the description of preferred codons or less preferred condons are “non-preferred codons.”

Accordingly, Seed's approach demands the use of the one specific codon prescribed in each instance and the replacement of every codon or nearly every codon in a sequence.

Likewise, the technology described by Pavlakis requires identification of inhibitory/instability sequences and the alteration of those specifically identified inhibitory/instability sequences. According to Pavlakis, an inhibitory/instability sequence of a transcript is a regulatory sequence that resides within an mRNA transcript and is either (1) responsible for rapid turnover of that mRNA and can destabilize a second indicator/reporter mRNA when fused to that indicator/reporter mRNA, or is (2) responsible for underutilization of a mRNA and can cause decreased protein production from a second indicator/reporter mRNA when fused to that second indicator/reporter mRNA or (3) both of the above. The procedures to locate and mutate the inhibitory/instability sequences are described in detail by Pavlakis. Accordingly, this approach is experimental result-dependent in that it requires preliminary experimentation to identify specific regions of sequence for targeted mutation.

Polynucleotide compositions that provide enhanced gene expression while obviating any requirement to alter each codon to a “preferred codon” or identify “inhibitory sequences” provide certain benefits. These benefits include not only improved efficiency, cost-effectiveness, consistency and accuracy in improving the expression of certain genes, but also the ability to achieve a far greater scope of applicability (i.e., the ability to attain such improved gene expression possible for genes for which it was previously not possible (or at least highly inefficient) using previously available technology). It would be desirable to have an approach to attain enhanced gene expression that avoids the stringent requirements of previous approaches. Accordingly, it would be desirable to have an approach to attain enhanced gene expression without having to alter all the codons of the gene to preferred codons or identify inhibitory sequences of the gene and then altering those sequences. Moreover, it would be desirable to have an approach that does not target, define, nor rely upon a specific transcriptional or translational mechanism for improved gene expression.

### SUMMARY OF THE INVENTION

The present invention provides enhanced gene expression in mammalian cells. In particular, the present invention provides modified polynucleotides with significantly improved expression over their wild-type counterparts. The present invention also provides compositions for preventing and treating conditions, as well as compositions for use in assays, vectors, diagnostic tools and the like.

According to an embodiment, the present invention provides a method of preventing or treating a disease in a mammal comprising: administering to the mammal an effective amount of one or more compositions of the invention.

According to a further embodiment, the present invention provides a method for enhancing expression of a gene comprising: expressing in vivo or in vitro a modified polynucleotide of the invention.

According to another embodiment, the present invention provides a method for preparing a polynucleotide that provides enhanced expression of a gene comprising: assembling oligonucleotides comprising surrogate codons to form a modified polynucleotide comprising a predetermined nucleic acid sequence wherein the nucleotides cytosine (C) or guanine (G) occupy the wobble position of each of said surrogate codons in place of the corresponding nucleotides adenine (A), uracil (U) or thymine (T) of a naturally-occurring polynucleotide that expresses the same protein or polypeptide as said modified polynucleotide.

According to yet another embodiment, the present invention provides a method for preparing a polynucleotide that provides enhanced expression of a gene comprising: (1) determining for said gene a modified nucleic acid sequence comprising surrogate codons in which the nucleotides cytosine (C) or guanine (G) occupy the wobble position in place of the corresponding nucleotides adenine (A) or uracil (U) or thymine (T) of a naturally-occurring polynucleotide that expresses the same protein or polypeptide as said modified polynucleotide; (2) selecting oligonucleotides having nucleotide sequences corresponding to portions of said determined recombinant nucleic acid sequence; and (3) assembling the oligonucleotides to form a recombinant polynucleotide comprising the determined recombinant nucleic acid sequence.

According to a still further embodiment, the present invention provides a method for enhancing expression of a gene comprising: altering a wild-type polynucleotide so that a naturally-occurring codon having adenine (A), uracil (U) or thymine (T) in the wobble position is replaced by a surrogate codon having cytosine (C) or guanine (G) in the wobble position, said surrogate codon encoding the same amino acid as the naturally-occurring codon.

According to another embodiment, the present invention provides a modified polynucleotide comprising a nucleic acid sequence comprising surrogate codons in which the nucleotides cytosine (C) or guanine (G) occupy the wobble position in place of the corresponding nucleotides adenine (A) or uracil (U), in RNA, or adenine (A) or thymine (T), in DNA, of a naturally-occurring polynucleotide that expresses the same protein or polypeptide as said modified polynucleotide.

According to a further embodiment, the present invention provides a modified polynucleotide comprising a nucleic acid sequence in which each codon encoding alanine is GCG, each codon encoding arginine is CGG or AGG, each codon encoding leucine is CTC, each codon encoding proline is CCT or CCG, each codon encoding glutamic acid is GAG, each codon encoding glycine is GGG, each codon encoding isoleucine is ATT, each codon encoding serine is TCC, each codon encoding threonine is ACG, and each codon encoding valine is GTC.

According to still another embodiment, the present invention provides a modified polynucleotide comprising a nucleic acid sequence having the general formula: —(X)ᵢ—(Y)ⱼ—(X)ᵢ—, wherein X represents non-surrogate codons having the nucleic acid sequence of any of the corresponding wild-type codons in the naturally-occurring polynucleotide that encode the same protein or polypeptide as said recombinant polynucleotide, said wild-type codons having cytosine (C) or guanine (G) in the wobble position, wherein Y represents surrogate codons having a nucleic acid sequence that is different from the corresponding wild-type codons in the naturally-occurring polynucleotide that encode the same protein or polypeptide as said recombinant polynucleotide, said wild-type codons having adenine (A) or uracil (U) or thymine (T) in the wobble position, said surrogate codons having cytosine (C), guanine (G) or thymine (T) in the wobble position and encoding the same amino acid as the corresponding wild-type codons in the naturally-occurring polypeptide that encodes the same protein or polypeptide as said modified polynucleotide, wherein i is any positive integer of at least 0; and wherein j is any positive integer of at least 1.

According to a still further embodiment, the present invention provides a modified polynucleotide comprising: (a) the nucleic acid sequence of any of SEQ ID NOS: 1, 3 or 5; (b) an immunogenic encoding portion of (a); or (c) a nucleic acid sequence that hybridizes under stringent conditions to any of (a) or (b).

According to another embodiment, the present invention provides a composition comprising: a modified polynucleotide comprising a nucleic acid sequence in which the nucleotides cytosine (C) or guanine (G) occupy the wobble position of surrogate codons in place of the corresponding nucleotides adenine (A), thymine (T) or uracil (U) in the nucleic acid sequence of a naturally-occurring polynucleotide that expresses the same protein or polypeptide as said recombinant polynucleotide; and a pharmaceutically acceptable buffer, diluent, adjuvant, carrier and/or vector.

According to yet another embodiment, the present invention provides a composition comprising a modified polynucleotide comprising a nucleic acid sequence in which each codon encoding alanine is GCG, each codon encoding arginine is CGG or AGG, each codon encoding leucine is CTC, each codon encoding proline is CCT or CCG, each codon encoding glutamic acid is GAG, each codon encoding glycine is GGG, each codon encoding isoleucine is ATT, each codon encoding serine is TCC, each codon encoding threonine is ACG, and each codon encoding valine is GTC; and a pharmaceutically acceptable buffer, diluent, adjuvant, carrier and/or vector.

According to a further embodiment, the present invention provides a composition comprising a pharmaceutically acceptable buffer, diluent, adjuvant, carrier and/or vector; and a modified polynucleotide comprising a nucleic acid sequence having the general formula: —(X)ᵢ—(Y)ⱼ—(X)ᵢ—; wherein X represents non-surrogate codons having the nucleic acid sequence of any of the corresponding wild-type codons in the naturally-occurring polynucleotide that encode the same protein or polypeptide as said modified polynucleotide, said wild-type codons having cytosine (C) or guanine (G) in the wobble position; wherein Y represents surrogate codons having a nucleic acid sequence that is different from the corresponding wild-type codons in the naturally-occurring polynucleotide that encode the same protein or polypeptide as said modified polynucleotide, said wild-type codons having adenine (A), uracil (U) or thymine (T) in the wobble position, said surrogate codons having cytosine (C) or guanine (G) in the wobble position and encoding the same amino acid as the corresponding wild-type codons in the naturally-occurring polynucleotide that encodes the same protein or polypeptide as said modified polynucleotide; wherein i is any positive integer of at least 0; and wherein j is any positive integer of at least 1.

According to another embodiment, the present invention provides a composition comprising: (a) the nucleic acid sequence of any of SEQ ID NOS: 1, 3 or 5; (b) an immunogenic encoding portion of (a); or (c) a nucleic acid sequence that hybridizes under stringent conditions to any of (a) or (b).

According to a still further embodiment, the present invention provides a composition comprising a polynucleotide comprising the nucleic acid sequence of any of SEQ ID NOS: 1, 3 or 5; and a vector.

According to another embodiment, the present invention provides a composition comprising: a recombinantly expressed protein or polypeptide encoded by a modified polynucleotide comprising any of: (a) the nucleic acid sequence of any of SEQ ID NOS: 1, 3 or 5; (b) an immunogenic encoding portion of (a); or (c) a nucleic acid sequence that hybridizes under stringent conditions to any of (a) or (b).

According to yet another embodiment, the present invention provides a composition comprising a recombinantly expressed protein or polypeptide encoded by a modified polynucleotide comprising a nucleic acid sequence comprising surrogate codons in which the nucleotides cytosine (C) or guanine (G) occupy the wobble position in place of the corresponding nucleotides adenine (A), uracil (U) or thymine (T) of a naturally-occurring polynucleotide that expresses the same protein or polypeptide as said recombinant polynucleotide.

According to a further embodiment, the present invention provides a composition comprising an antibody that immunospecifically binds to a recombinantly expressed protein of the invention.

According to an even further embodiment, the present invention provides a composition prepared by a process comprising inserting into a vector a modified nucleic acid sequence comprising surrogate codons in which the nucleotides cytosine (C) or guanine (G) occupy the wobble position in place of the corresponding nucleotides adenine (A), uracil (U) or thymine (T) of a naturally-occurring polynucleotide that expresses the same protein or polypeptide as said modified polynucleotide.

According to a still further embodiment, the present invention provides a composition prepared by a process comprising: inserting into a vector a modified nucleic acid sequence in which each codon encoding alanine is GCG, each codon encoding arginine is CGG or AGG, each codon encoding leucine is CTC, each codon encoding proline is CCT or CCG, each codon encoding glutamic acid is GAG, each codon encoding glycine is GGG, each codon encoding isoleucine is ATT, each codon encoding serine is TCC, each codon encoding threonine is ACG, and each codon encoding valine is GTC.

According to another embodiment, the present invention provides a composition prepared by a process comprising: inserting into a vector a polynucleotide comprising a modified nucleic acid sequence having the general formula: —(X)ᵢ—(Y)ⱼ—(X)ᵢ—; wherein X represents non-surrogate codons having the nucleic acid sequence of any of the corresponding wild-type codons in the naturally-occurring polynucleotide that encode the same protein or polypeptide as said modified polynucleotide, said wild-type codons having cytosine (C) or guanine (G) in the wobble position; wherein Y represents surrogate codons having a nucleic acid sequence that is different from the corresponding wild-type codons in the naturally-occurring polynucleotide that encode the same protein or polypeptide as said modified polynucleotide, said wild-type codons having adenine (A) or uracil (U) in the wobble position, said surrogate codons having cytosine (C), guanine (G) or thymine (T) in the wobble position and encoding the same amino acid as the corresponding wild-type codons in the naturally-occurring polypeptide that encodes the same protein or polypeptide as said modified polynucleotide; wherein i is any positive integer of at least 0; and wherein j is any positive integer of at least 1.

According to yet another embodiment, the present invention provides a composition prepared by a process comprising: inserting into a vector any of: (a) the nucleic acid sequence of any of SEQ ID NOS: 1, 3 or 5; (b) an immunogenic encoding portion of (a); or (c) a nucleic acid sequence that hybridizes under stringent conditions to any of (a) or (b).

According to a further embodiment, the present invention provides for the use of a composition in the preparation of a medicament for inducing an immune response in a mammal.

According to another embodiment, the present invention provides for the use of a composition in the preparation of a medicament for treating a condition in a mammal.

According to a still further embodiment, the present invention provides a transformed, transfected, lipofected or infected cell line comprising: a recombinant cell that expresses any of: (a) the nucleic acid sequence of any of SEQ ID NOS: 1, 3 or 5; (b) an immunogenic encoding portion of (a); or (c) a nucleic acid sequence that hybridizes under stringent conditions to any of (a) or (b).

According to another embodiment, the present invention provides a modified polynucleotide comprising: (a) the nucleic acid sequence of any of SEQ ID NOS: 12-16; (b) an immunogenic encoding portion of (a); or (c) a nucleic acid sequence that hybridizes under stringent conditions to any of (a) or (b).

According to yet another embodiment, the present invention provides a composition that comprises a modified polynucleotide comprising: (a) a non-native leader sequence; and (b) a nucleic acid sequence comprising cytosine (C) or guanine (G) at the wobble position of at least one codon that encodes any of the amino acids alanine, arginine, leucine, proline, glutamic acid, glycine, isoleucine, serine, threonine, or valine where adenine (A), uracil (U) or thymine (T) occupy the wobble position of the corresponding codon of the naturally-occurring nucleic acid sequence.

According to a further embodiment, the present invention provides a composition that comprises a recombinant polynucleotide comprising: (a) an IgE leader sequence; and (b) a nucleic acid sequence comprising cytosine (C) or guanine (G) at the wobble position of at least one codon that encodes any of the amino acids alanine, arginine, leucine, proline, glutamic acid, glycine, isoleucine, serine, threonine, or valine where adenine (A), uracil (U) or thymine (T) occupy the wobble position of the corresponding codon of the naturally-occurring nucleic acid sequence.

According to a still further embodiment, the present invention provides a composition comprising: a polynucleotide comprising (a) a nucleic acid sequence having at least about 70% sequence identity to the nucleic acid sequence of SEQ ID NO:14; or (b) a nucleic acid sequence that hybridizes to SEQ ID NO:14 under stringent conditions.

### BRIEF DESCRIPTION OF THE DRAWINGS

FIG. 1 is a graph comparing the expression of protein from the recombinant HIV-1 6106 env gp160 gene prepared in accordance with an embodiment of the present invention relative to the expression of protein from the wild-type gp160 gene and gp160 gene having modified inhibitory sequences.

FIG. 2 is a plasmid map of the plasmid construct of SEQ ID NO:7.

FIG. 3 is a plasmid map of the plasmid construct of SEQ ID NO:8.

FIG. 4 is a plasmid map of the plasmid construct of SEQ ID NO:9.

FIG. 5 is a plasmid map of the plasmid construct of SEQ ID NO:10.

FIG. 6 is a graph comparing expression of protein from IL-15 modified polypeptide (LP) with an IgE leader sequence in accordance with an embodiment of the present invention relative to the expression of protein from alternative IL-15 constructs in (a) RD cells; (b) COS7 cells, and (c) Hela cells.

FIG. 7 is a graph comparing expression of protein from IL-15 modified polypeptide (LP) with an IgE leader sequence in accordance with an embodiment of the present invention relative to the expression of protein from alternative IL-15 constructs in (a) RD cells, and (b) 293 cells.

FIG. 8 is a table comparing expression (fold increase) of protein from IL-15 modified polypeptide (LP) with an IgE leader sequence in accordance with an embodiment of the present invention relative to the expression of protein from alternative IL-15 constructs in RD cells, COS7 cells, Hela cells, and 293 cells.

FIG. 9 is a graph comparing expression of protein from IL-15 modified polypeptide (LP) with an IgE leader sequence in accordance with an embodiment of the present invention relative to the expression of protein from alternative IL-15 constructs in a CTLL2 mouse cell proliferation assay.

FIG. 10 is a graph comparing in vivo expression of protein from IL-15 modified polypeptide (LP) with an IgE leader sequence in accordance with an embodiment of the present invention relative to the expression of protein from alternative IL-15 over time.

FIG. 11 is a plasmid map for the O-IL-15-IgE leader plasmid construct according to an embodiment of the present invention.

FIG. 12 is a plasmid map for the LP-IL-15-IgE leader plasmid construct according to an embodiment of the present invention.

FIG. 13 is a plasmid map for the BH-15-IgE leader plasmid construct according to an embodiment of the present invention.

### DETAILED DESCRIPTION OF THE INVENTION

An appropriate level of a protein in mammalian cells is essential in vivo for enhanced immunological and/or therapeutic responses, e.g., the use of the gene and its protein product as an immunogen, DNA vaccine, co-immunogen, adjuvant, carrier protein or vector, therapeutic agent, diagnostic agent, therapeutic, immuno-prophylactic, immuno-therapeutic, etc., as well as for in vitro recombinant protein expression purposes, e.g., the use of the gene and its protein product in assays, tests, diagnostics, research tools, etc. The efficiency of a gene in expressing its protein product is a controlling factor in the attainment of appropriate levels of the protein in cells. Certain wild-type genes fail to provide appropriate protein levels in mammalian cells. The present invention is directed to improving the expression efficiency of such genes.

An effective IL-15 plasmid for DNA vaccination that secretes enhanced levels of IL-15 was unexpectedly identified. In particular, it was found that 1) the replacement of native signal peptide with the Human IgE leader sequence; 2) non preferred codons are replaced with either optimized or less preferred codons while preserving the native amino acid sequence; 3) the nucleotide sequence was modified to reduce the secondary mRNA structure for improved translation.

### Modified Polynucleotides

As described herein, the inventors have devised modified polynucleotides that provide unexpectedly improved gene expression in mammalian cells both in vitro and in vivo for various poorly-expressed genes.

These polynucleotides represent a new version of a wild-type gene. In particular, the inventors discovered that enhanced expression was unexpectedly provided by a new version of a gene in the form of a synthesized polynucleotide which comprises “surrogate codons” in the open reading frame (ORF) of the gene sequence, wherein the “surrogate codons” still encode identical amino acid residues (although biologically equivalent amino acid sequences/proteins, substantially identical amino acid sequences/proteins, etc. are also contemplated by the present invention, as described in further detail below).

A “surrogate codon”, as used herein, refers to a codon for an ORF, other than the naturally occurring (i.e., wild-type) codon when that wild-type codon has an A, T (in the case of DNA) or U (in the case of RNA) in the wobble position, but encoding the same amino acid as that corresponding naturally occurring codon (i.e., the codon at the same position in the wild-type ORF). As used herein, the terms, “naturally-occurring and “wild-type” are used interchangeably herein. In certain embodiments, the surrogate codon has C or G in its wobble position. In another embodiment, the surrogate codon is not a “preferred codon” as defined by Seed et al. The surrogate codons of the present invention are used in modified polynucleotides in place of corresponding disfavored codons, e.g., the naturally-occurring codon with A or T (if DNA) or U (if RNA) in the wobble position, of the wild-type form of the gene, for certain of the amino acids as described below. As used herein, the “wobble” position of a codon is the third nucleotide position of a codon triplet, as read in the 5′ to 3′ direction.

The invention disclosed herein utilizes a general approach directed to modified forms of a gene (i.e., recombinant polynucleotides). According to this general approach, modified polynucleotides are formed. These polynucleotides comprise a nucleic acid sequence comprising surrogate codons in place of at least some of the codons of the corresponding wild-type polynucleotide for the gene. For example, in accordance with embodiments of the invention, a modified polynucleotide comprises a nucleic acid sequence comprising surrogate codons in which the nucleotides cytosine (C) or guanine (G) occupy the wobble position in place of the corresponding nucleotides adenine (A) or uracil (U) or thymine (T) of a naturally-occurring polynucleotide that expresses substantially the same protein or polypeptide as said modified polynucleotide (or a functionally equivalent protein or polypeptide, as would be known to a person of skill in the art). The modified polynucleotide of the invention need not be an exact replica of the wild-type ORF wherein every codon having A or U in the wobble position is substituted with a surrogate codon. Merely a sufficient number of surrogate codons in place of naturally occurring codons to achieve enhanced gene expression is necessary.

A minimally sufficient number of surrogate codons or any number greater than that amount is contemplated by the invention. A suitable number of surrogate codons for a polynucleotide in accordance with the present invention is readily determined by one of skill by routine testing. It is not necessary that a predetermination of a specific number of surrogate codons be made. However, a predetermined number of replacements may be used in the interest of efficiency. For example, in constructing a polynucleotide of the invention, one may predetermine that a specified percentage of the codons of the ORF may be re-engineered, for example, about 5%, 10%, 20%, 30%, 40%, 50%, 60%, 70%, 80%, 90%, 95%, 96%, 97%, 98%, 99% or 100% of the codons, without limitation, may be the subject of re-engineering. Normally, at least 10% of the codons are the subject of reengineering (e.g., 10% of the ORF is the new version of the gene while the remaining 90% is the same as or functionally the same as the wild-type ORF). In certain embodiments, at least about 50% of the codons are the subject of re-engineering. In other embodiments, at least about 90% of the codons are the subject of re-engineering with surrogate codons.

The surrogate codons of the present invention are the non-naturally-occurring codons (of a gene) that encode for the following amino acids: alanine (Ala), asparagine or aspartate (Asx), cysteine (Cys), aspartate (Asp), glutamate (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), tyrosine (Tyr), or glutamine or glutamate (Glx). In a particular embodiment, the surrogate codons of the invention are the non-naturally-occurring codons (of a gene) with C or G in the wobble position that encode for any of alanine (Ala), asparagine or aspartate (Asx), cysteine (Cys), aspartate (Asp), glutamate (Glu), phenylalanine (Phe), glycine (Gly), histidine (His), lysine (Lys), leucine (Leu), methionine (Met), asparagine (Asn), proline (Pro), glutamine (Gln), arginine (Arg), serine (Ser), threonine (Thr), tyrosine (Tyr), or glutamine or glutamate (Glx), without limitation. A recombinant polynucleotide of the invention need not include surrogate codons for each amino acid encoded. Select surrogate codons that encode any number of amino acids may be predetermined for inclusion in the recombinant version of the gene provided that the objective of improving expression of the gene is achieved. A person of skill in the art would be able to determine through routine testing a minimally effective number. In one particular embodiment, each of the codons for alanine (Ala), asparagine or aspartate (Asx), cysteine (Cys), aspartate (Asp), glutamate (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), tyrosine (Tyr), or glutamine or glutamate (Glx) is replaced with a surrogate codon to form the recombinant version of the gene in accordance with an embodiment of the invention.

Accordingly, in the present invention, it is unnecessary to replace each codon that has A, T or U in the wobble position for every amino acid, substitute in specifically determined “preferred codons” or remove inhibitory sequences.

In certain embodiments, the surrogate codons used in the modified polynucleotides of the present invention are those that encode alanine, arginine, leucine, proline, glutamic acid, glycine, isoleucine, serine, threonine and valine. In other embodiments, the surrogate codons used in the polynucleotides of the invention are those that encode alanine, arginine, leucine, proline, glycine, isoleucine, serine, threonine and valine. In one particular embodiment, the surrogate codons used in the modified polynucleotides of the invention are those that encode alanine, arginine, leucine, proline, glycine, serine, threonine and valine.

In accordance with an embodiment of the invention, the surrogate codons are a randomized selection of at least about 10% of the codons in said modified polynucleotide that encode for any of the amino acids alanine, arginine, leucine, proline, glycine, isoleucine, serine, threonine and valine. In accordance with another embodiment, the surrogate codons are a randomized selection of at least about 50% of the codons in said polynucleotide that encode for any of the amino acids alanine, arginine, leucine, proline, glycine, isoleucine, serine, threonine and valine. In a further embodiment, the surrogate codons are a randomized selection of at least about 90% of the codons in said polynucleotide that encode for any of the amino acids alanine, arginine, leucine, proline, glycine, isoleucine, serine, threonine and valine. In yet another embodiment, the surrogate codons are each of the codons in said polynucleotide (i.e., 100%) that encode for the amino acids alanine, arginine, leucine, proline, glycine, isoleucine, serine, threonine and valine.

The present invention contemplates embodiments directed to any gene that is poorly expressed or any gene for which improved levels of protein expression is desirable for in vivo and/or in vitro uses. For example, a subject gene may be a viral, bacterial, protist, fungal, plant or animal gene, without limitation. Any such gene that is poorly expressed in mammalian cells is contemplated by the present invention.

In the case of viral genes, without limitation, the viral gene may be associated with a DNA (double stranded or single stranded) or RNA (double stranded or single stranded) virus, without limitation. Viral genes of viruses from any viral family are contemplated by the present invention, including, for example, Adenoviridae, Arenaviridae, Arterivirus, Astroviridae, Baculoviridae, Badnavirus, Barnaviridae, Brinaviridae, Bromoviridae, Bunyaviridae, Caliciviridae, Capillovirus, Carlavirus, Caulimovirus, Circoviridae, Closteroviridae, Comoviridae, Coronaviridae, Corticoviridae, Cystoviridae, Deltavirus, Dianthovirus, Enamovirus, Filoviridae, Flaviviridae, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteovirus, Machlomovirus, Marafivirus, Microviridae, Myoviridae, Necrovirus, Nodaviridae, Orthomyxoviridae, Papovaviridae, Paramyxoviridae, Partitiviridae, Parvaviridae, Phycodnaviridae, Picornaviridae, Plasmaviridae, Podoviridae, Polydnaviridae, Potexvirus, Potyviridae, Poxyiridae, Reoviridae, Retroviridae, Rhabdoviridae, Rhizidiovirus, Sequiviridae, Siphoviridae, Sobemovirus, Tectiviridae, Tenuivirus, Tetraviridae, Tobamovirus, Tobravirus, Togavridae, Tombusviridae, Totiviridae, Trichovirus, Tymovirus, Umbravirus, Viroids, Mononegavirales, Tailed Phages, and as yet unclassified viruses, without limitation.

In one embodiment of the invention, a viral gene is associated with lentiviruses, retroviruses, herpes viruses, adenoviruses, adeno-associated viruses, vaccinia virus, or baculovirus, without limitation. In certain embodiments, viral genes include, for example, those of Human immunodeficiency virus, Simian immunodeficiency virus, Respiratory syncytial virus, Parainfluenza virus types 1-3, Influenza virus, Herpes simplex virus, Human cytomegalovirus, Hepatitis A virus, Hepatitis B virus, Hepatitis C virus, Human papillomavirus, poliovirus, rotavirus, caliciviruses, Measles virus, Mumps virus, Rubella virus, adenovirus, rabies virus, vesicular stomatitis virus, canine distemper virus, rinderpest virus, Human metapneumovirus, avian pneumovirus (formerly turkey rhinotracheitis virus), Hendra virus, Nipah virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus and various Encephalitis viruses, without limitation.

Specific viral genes contemplated by the present invention include, for example, any of the genes of HIV or any of the genotypes of HPV, including high-risk and low-risk genotypes. For example, genes of HIV contemplated by the invention include gag, pol, env, tat, rev, vif, nef, vpr, vpu and vpx, without limitation. Genes of HPV contemplated by the invention include, for example, E1, E2, L1, L2, E6 and E7 without limitation. The genotypes of HPV contemplated by the present invention include, for example, high-risk genotypes, such as HPV 16, 18, 31, 33, 45, 52, 56 or 58 and low-risk genotypes, such as 6 and 11, without limitation. According to an embodiment, the gene is the human papillomavirus 16 (HPV16) E7 gene (E7), or human immuno-deficiency virus (HIV-1) gag gene (gag) or gp160 envelope gene (env). Compositions, fusion constructs or any other multi-gene structures containing any combination of the foregoing are also contemplated by the present invention.

Specific bacterial genes include the genes of any bacterial species, including for example, without limitation, Haemophilus influenzae (both typable and nontypable), Haemophilus somnus, Moraxella catarrhalis, Streptococcus pneumoniae, Streptococcus pyogenes, Streptococcus agalactiae, Streptococcus faecalis, Helicobacter pylori, Neisseria meningitidis, Neisseria gonorrhoeae, Chlamydia trachomatis, Chlamydia pneumoniae, Chlamydia psittaci, Bordetella pertussis, Alloiococcus otiditis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium-Mycobacterium intracellulare complex, Proteus mirabilis, Proteus vulgaris, Staphylococcus aureus, Staphylococcus epidermidis, Clostridium tetani, Leptospira interrogans, Borrelia burgdorferi, Pasteurella haemolytica, Pasteurella multocida, Actinobacillus pleuropneumoniae and Mycoplasma gallisepticum.

Further, the present invention is applicable to any gene which is a suitable subject for improved efficiency in the manner of the present invention, i.e., engineering a recombinant polynucleotide for the gene with surrogate codons in place of naturally occurring codons with A or U in the wobble position. Thus, although the term “poorly-expressed” genes is used throughout, the present invention is by no means intended to be limited to genes that meet some threshold requirement of poor expression. Instead, modified polynucleotides directed to poorly-expressed genes are merely exemplary to illustrate the dramatic improvement in protein levels in the circumstances where such improvement is most pertinent. Therefore, the present invention contemplates applicability to genes that may not be considered to be poorly-expressed by persons skilled in the art, as well as to those that are generally considered or proven to be poorly-expressed, without limitation.

Upon selection of a desired target gene of a desired species (e.g., the E1 gene of HPV 16), a person of skill in the art, based upon the guidance provided herein, would be able to formulate the sequence of a desired recombinant in accordance with an embodiment of the present invention. The sequencing is performed for example, by hand or is computer-assisted. A person of skill in the art may make a replacement at each disfavored wobble position, or at some percentage of the disfavored wobble positions. For example, the first 50% of disfavored wobble positions or the second 50% of disfavored wobble positions. The modified sequence is tested by routine methods to determine whether the percentage change provides a desired level of expression. The examples herein provide guidance as to such testing, however, it is well within the abilities of a person of skill in the art to conduct such routine testing in a variety of ways. In certain embodiments, replacement is made at each disfavored wobble position, thus eliminating the need to select certain portions of the gene and certain percentages of wobble positions for replacement. Once the sequence of the polynucleotide is determined, it is well within the ability of a person of skill in the art to prepare the modified polynucleotide using well known techniques and methods, as further described in the examples below.

Several poorly-expressed viral genes illustrate the benefits of the present invention. For example, the following wild-type viral genes demonstrate poor expression in mammalian cells: human papillomavirus 16 (HPV16) E7, human immuno-deficiency virus type-1 (HIV-1) gag and gp160 (envelope) (hereafter denoted E7, gag, and env, respectively). In each of these wild-type genes, the naturally occurring nucleic acid sequences of the genes are AU rich and biased toward “disfavored codons” (containing an A or U in the 3d or “wobble” position of the codon nucleotide triplet). As noted above, mammalian genes that express proteins at high levels have a G/C preference in the wobble position. Thus, these wild-type genes with A or U in the wobble position may not be handled efficiently by the mammalian translational machinery.

Further, as discussed above, separately from the translational mechanisms accounting for poorly-expressed genes, there have been various AU rich RNA instability sequences discovered in several messenger RNAs (mRNAs) which do not directly impact the translatability of a given mRNA but limit protein expression by increasing mRNA turnover. In addition, several specific “inhibitory” sequences contained within the HIV-1 gag ORF have been described (see Pavlakis) which limit the expression levels of gag by inhibiting nuclear export of these transcripts. Codons encompassing these inhibitory sequences are difficult to selectively replace to eliminate the inhibitory effect because the sequence motifs that define either instability or inhibitory sequences are not easily identified. Moreover, several genes (e.g. E7 and En among others) which appear to also contain inhibitory sequences have not yet been mapped to identify the location of inhibitory sequences and there are no straightforward prescriptions from the gag work to predict how to eliminate inhibitory sequences from these genes.

According to an embodiment of the present invention, codons throughout a gene sequence are replaced (e.g., surrogate codons replace wild-type codons in a modified construct) without the need to identify and then mutate inhibitory sequences (as performed for gag) and without altering every codon by use of preferred codons (as performed for env). When a naturally occurring disfavored codon (e.g., with A or U in the wobble position) is replaced with (i.e., its position in the modified form is occupied by) a “surrogate codon” encoding the same amino said, there is an opportunity to eradicate inhibitory sequence(s), instability sequence(s), and/or provide codons that are more efficiently translated than their naturally occurring counterparts.

It was surprisingly discovered that alteration of all possible codons and utilization of “preferred” codons was not necessary to achieve improved protein levels expressed by the genes cited above. Thus, it is possible to exploit the degeneracy of the genetic code to develop recombinant polynucleotides with improved protein expression of a gene relative to the wild-type polynucleotide of the gene (or other recombinant polynucleotides for the gene). Thus, it is unnecessary to construct a complete “codon optimized” version of gp120 envelope as previously described (see Haas et al., Andre et al.) in which non-preferred wild-type codons from env were replaced with “preferred” codons to enhance protein levels expressed by the gene.

Table I below lists non-limiting examples of surrogate codons of the present invention. In particular, Table I shows the surrogate codons for ten of the twenty L-amino acids that have been utilized as replacements for existing disfavored codons, according to an implementation of the present invention. In accordance with this embodiment of the invention, codons encoding the remaining ten amino acids were not replaced by surrogate codons in the modified form of the gene.

TABLE I
SURROGATE CODONS

| | Amino acid | | Amino acid |
|---|---|---|---|
| Codon | encoded | Codon | encoded |
| GCG | Alanine | GAG | Glutamic Acid |
| CGG or AGG | Arginine | GGG | Glycine |
| CTC | Leucine | ATT | Isoleucine |
| CCT or CCG | Proline | TCC | Serine |
| ACG | Threonine | GTC | Valine |

In accordance with an embodiment of the present invention, recombinant polynucleotides were prepared in which disfavored codons (A or U at the wobble position) were replaced by the surrogate codons listed in Table I above for the amino acid encoded by the disfavored codon, and the corresponding new (i.e. modified) nucleic acid sequence was created by joining oligonucleotides encoding the new sequence and assembling the fragments to create the modified polynucleotide comprising the new sequence.

The recombinant ORF was cloned into a plasmid DNA expression vector that allowed in vitro expression-studies for comparing the levels of protein expression of the modified polynucleotide and the wild-type polynucleotide. Transient transfection assays (data not shown) performed with several cell lines revealed increases in protein expression levels for three gene products (i.e., E7, gag, and env) when their gene sequence was modified as described above. The increased protein expression (as measured by Western blot, ELISA and the like) demonstrated by the altered codon constructs compared to wild-type (naturally occurring) construct for three different genes indicated that this method is applicable to a variety of poorly expressed proteins.

In recognition that several codon choices are possible for some of the twenty amino acids, for example, the amino acids alanine, arginine, glycine, glutamic acid, isoleucine, leucine, proline, serine, threonine, and valine, an embodiment of the present invention is directed to the codons encoding those amino acids. Thus, in accordance with an embodiment of the invention, a modified polynucleotide has a nucleic acid sequence, which differs from that of the wild-type sequence, in which each codon, that corresponds to a naturally-occurring codon having A, U or T in the wobble position, encoding alanine is GCG, each codon encoding arginine is CGG or AGG, each codon encoding leucine is CTC, each codon encoding proline is CCT or CCG, each codon encoding glutamic acid is GAG, each codon encoding glycine is GGG, each codon encoding isoleucine is ATT, each codon encoding serine is TCC, each codon encoding threonine is ACG, and each codon encoding valine is GTC.

In certain other embodiments, codons for amino acids other than the ten listed above also serve as surrogate codons. In other words, replacement of the naturally-occurring codons, with A, U or T in the wobble position, encoding other amino acids is contemplated. It is also contemplated that certain embodiments of the invention provide surrogate codons for only some of the ten amino acids listed in Table I. Upon grasping the concept of the invention as fully described herein, a person skilled in the art would routinely be able to determine a minimally or optimally desired number of codons through routine methods, based upon the guidance provided herein. In certain embodiments, the polynucleotides of the present invention comprise surrogate codons for just the nine amino acids, alanine, arginine, glycine, isoleucine, leucine, proline, serine, threonine, and valine in place of each of the corresponding codons having A or U in the wobble position. It should be noted, however, that any changes to those changed codons and/or the other codons that permit the protein to retain its functionality are contemplated by the present invention. Examples of such changes are provided below.

The modified polynucleotides of the invention are prepared in any suitable manner as would be known to persons skilled in the art. For example, the present invention contemplates the use of chemical synthesis, nucleotide substitution, codon substitution, DNA libraries, mutagenesis, isolation and purification from native entity, etc. and any combinations thereof, without limitation.

In one embodiment, a full length polynucleotide sequence is determined by selecting surrogate codons for the disfavored codons. This may be done by hand, computer-assisted or any other method. Once the desired sequence is determined, then oligonucleotides comprising fragments of the determined sequence are obtained or prepared. Such oligonucleotides are readily obtained from commercial vendors, such as Invitrogen™ (Carlsbad, Calif.). The fragments are selected such that they can form a staggered, overlapping arrangement. The modified polynucleotides are synthesized by joining oligonucleotides that comprise fragments of the recombinant nucleic acid sequence. The fragments are hybridized and subsequently filled in by a DNA polymerase (such as Pfx Turbo, Invitrogen). This staggered, overlapping arrangement of the fragments is then ligated, for example, using a heat stable ligase (Ampligase).

Specific protocols for preparing the polynucleotides of the present invention are provided in the Examples below. These specific protocols are merely illustrative. A person skilled in the art would readily be able to employ a variety of suitable techniques to accomplish the objectives of the present invention, upon grasping the inventive concepts disclosed herein. All such suitable techniques for preparing recombinant polynucleotides are contemplated by the present invention.

According to an embodiment of the invention, the leader sequence of the polynucleotide is altered or substituted with a non-native leaders sequence. For example, a non-native leader sequence is added to a modified polynucleotide of the presents invention and replaces the native leader sequence of the polynucleotide. Thus, the present invention contemplates a modified polynucleotide comprising a non-native leader sequence. The non-native leader sequence may be any suitable sequence or combination thereof that provides enhanced expression. It has been suprisingly found that the combination of modifying the polynucleotides using surrogate codons as described herein with the use of a non-native leader sequence provides synergistically improved expression, as described in Example 5 below. The non-native leader sequence may be human non-native leader sequence. The non-native leader sequence may be an immunoglobulin leader sequence.

According to an embodiment, the non-native leader sequence is (a) an IgE leader sequence or (b) a leader sequence that hybridizes to an IgE leader sequence under stringent conditions. According to another embodiment, the non-native leader sequence is: (a) a leader sequence having SEQ ID NO:11; or (b) a leader sequence that hybridizes to SEQ ID NO:11 under stringent conditions. The non-native leader sequence has at least 70%, 80%, 90%, 95%, 97%, 98% or 99% sequence identity to the nucleic acid sequence of SEQ ID NO:11 according to other embodiments of the present invention. According to another embodiment, the non-native leader sequence has the nucleic acid sequence of SEQ ID NO:11. A person skilled in the art would readily be able to construct or alter a polynucleotide to include a non-native leader sequence in the manner of the present invention, based upon the guidance provided herein.

The polynucleotides are prepared in various forms (e.g., single-stranded, double-stranded, vectors, probes, primers) as desired. The term “polynucleotide” includes any strand of DNA and RNA, single stranded and double stranded, and also their analogs, such as those containing modified backbones. The term “modified polynucleotide” as used herein, describes any strand of DNA or RNA, including single or double stranded, that are recombinantly prepared or that have been altered from their naturally-occurring state (through insertion, deletion, substitution, etc.) with surrogate codons or as otherwise consistent with the embodiments of the present invention as described herein. The DNA may be of any type, such as cDNA, genomic DNA, synthesized DNA, isolated DNA or a hybrid thereof. The RNA may be also be of any type RNA molecule such as mRNA. The constructs of the present invention contemplate any regulator elements necessary or desirable for expression of the sequence, such as a promoter, an initiation codon, a stop codon, and a polyadenylation signal, for example, without limitation. Any suitable enhancer is also contemplated by the present invention. Non-limiting exemplary enhancers include human Actin, human Myosin, human Hemolobin, human muscle creatine, and viral enhancers such as those from CMV, RSV and EBV.

Several specific recombinant polynucleotides, including specific nucleic acid sequences, for various viral genes are provided herein. These are merely exemplary and the invention is not intended to be limited thereto. Rather, the inventive concept is broadly applicable as described herein. Moreover, the present invention contemplates modified polynucleotides which are variations on any of the recombinant polynucleotides described herein, such as, for example, the specifically disclosed sequences, without limitation. For example, these would include variations wherein the variant nucleic sequence encodes a different amino acid sequence than the specifically disclosed sequence, however, the functionality of the different amino acid sequence is the same as that encoded by the sequence described herein.

According to an embodiment the modified polynucleotide expresses a viral polypeptide. The present invention contemplates modified polynucleotides from any agent or organism, such as pathogenic organisms, for example, HIV, HSV, HCV, WNV or HBV. For example, according to an embodiment immunogenic compositions are prepared from the pathogenic organisms for the purpose of immunizing an individual against the pathogen. For example, the modified polynucleotide may express the viral polypeptides HPV16 HIV-1 or gp160 or any combinations thereof, without limitation. According to an embodiment, a modified polynucleotide may comprise the ORF for HPV16 E7 gene. According to another embodiment, a modified polynucleotide comprises the ORF for the HIV-1 gag gene. According to another embodiment, a modified polynucleotide comprises the ORF for the gp160 envelope gene.

According to an embodiment, the modified polynucleotide encodes for a cytokine, growth factor, lymphokine, such as alpha-interferon, gamma-interferon, GM-CSF, platelet derived growth factor, TNF, EGF, ILA, IL-2, IL-4, IL-6, IL-10, IL-12, IL-15 as well as fibroblast growth factor, surface active agents such as immune-stimulating complexes (ISCOMS), Freunds incomplete adjuvant, LPS analog including monophsphoryl Lipid A (WL), muramyl peptides, quinone analogs and vesicles such as squalene and squalene, and hyaluronic acid. Any cytokine is contemplated by the present invention. According to another embodiment, the cytokine is an interleukin. According to another embodiment, polynucleotide encodes for IL-15 or a peptide or polypeptide having the activity of IL-15. According to another embodiment, the modified polynucleotide encodes for IL-15. According to another embodiment, the modified polynucleotide comprises the nucleic acid sequence of any of SEQ ID NOS: 12-16. According to another embodiment, the modified polynucleotide comprises the nucleic acid sequence of SEQ ID NO:14. The nucleotide and amino acid sequences of IL-15 are well known and set forth in Campbell, et al. (1987) Proc. Natl. Acad. Sci. USA 84:6629-6633, Tanabe, et al. (1987) J. Biol. Chem. 262:16580-16584, Campbell, et al. (1988) Eur. J. Biochem. 174:345-352, Azuma, et al. (1986) Nucl. Acids Res. 14:9149-9158, Yokota, et al. (1986) Proc. Natl. Acad. Sci. USA 84:7388-7392, and accession code Swissprot PO5113, which are each incorporated herein by reference in their entirety.

For example, according to an embodiment of the present invention, the modified polynucleotides comprise a nucleic acid sequence that is identical to any of the reference sequences of odd numbered SEQ ID NOS:1-5 or any of SEQ ID NOS:12-16 (which are sequences modified in accordance with the invention), that is 100% identical, or it may include a number of nucleotide alterations (e.g. at least 99%, 98%, 97%, 96%, 95%, 94%, 90%, 85%, 80%, 70%, or 60% identical, etc.) as compared to the reference sequence. Such alterations are selected from the group consisting of at least one nucleotide deletion, substitution, including transition and transversion, or insertion, and wherein said alterations occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in any of odd numbered SEQ ID NOS:1-5 or any of SEQ ID NOS:12-16 by the numerical percent of the respective percent identity (divided by 100) and subtracting that product from said total number of nucleotides in said sequence.

Certain embodiments of the invention relate to polynucleotides and sequence modifications thereof. In one embodiment, a polynucleotide of the invention is a polynucleotide comprising a nucleotide sequence having functional equivalency and at least about 95% identity to a nucleotide sequence chosen from one of the odd numbered SEQ ID NO:1-5 or any of SEQ ID NOS:12-16, a degenerate variant thereof, or a fragment thereof. As defined herein, a “degenerate variant” is defined as a polynucleotide that differs from the nucleotide sequence shown in the odd numbered SEQ ID NOS:1-5 or any of SEQ ID NOS:12-16 (and fragments thereof) due to degeneracy of the genetic code, but still encodes the same protein (e.g., the even numbered SEQ ID NOS: 2-6) as that encoded by the nucleotide sequence shown in the odd numbered SEQ ID NOS: 1-5 or any of SEQ ID NOS:12-16.

In other embodiments, the polynucleotide is a complement to a nucleotide sequence chosen from one of the odd numbered SEQ ID NOS: 1-5 or any of SEQ ID NOS:12-16, a degenerate variant thereof, or a fragment thereof. In yet other embodiments, the polynucleotide is selected from the group consisting of DNA, chromosomal DNA, cDNA and RNA and may further comprises heterologous nucleotides. In another embodiment, an isolated polynucleotide hybridizes to a nucleotide sequence chosen from one of odd numbered SEQ ID NOS: 1-5 or any of SEQ ID NOS:12-16, a complement thereof, a degenerate variant thereof, or a fragment thereof, under high stringency hybridization conditions. In yet other embodiments, the polynucleotide hybridizes under intermediate stringency hybridization conditions.

It will be appreciated that polynucleotides of the present invention are obtained from natural sources (and then altered) or are synthetic or semi-synthetic or some combination thereof. Furthermore, the nucleotide sequence is related by mutation, including single or multiple base substitutions, deletions, insertions and inversions, to a naturally occurring sequence, provided always that the nucleic acid molecule comprising such a sequence is capable of being expressed as a functionally equivalent polypeptide as described above. A nucleic acid molecule of the invention is RNA, DNA, single stranded or double stranded, linear or covalently closed circular form. In certain embodiments, the nucleotide sequence has expression control sequences positioned adjacent to it, such control sequences usually being derived from a heterologous source. In other embodiments, the recombinant expression of a nucleic acid sequence of the invention include a stop codon sequence, such as TAA, at the end of the nucleic acid sequence.

According to an embodiment, the invention also includes polynucleotides capable of hybridizing under reduced stringency conditions. According to another embodiment the invention includes polynucleotides capable of hybridizing under stringent conditions, and under another embodiment the present invention includes polynucleotides capable of hybridizing under highly stringent conditions, to the polynucleotides described above. Examples of stringency conditions are shown in the Stringency Conditions Table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE II
HYBRIDIZATION STRINGENCY CONDITIONS

| | Poly- | Hybrid | Hybridization | Wash |
|---|---|---|---|---|
| Stringency | nucleotide | Length | Temperature and | Temperature |
| Condition | Hybrid | (bp)I | BufferH | and BufferH |
| A | DNA:DNA | >50 | 65 C.; 1xSSC -or- | 65 C.; 0.3xSSC |
| | | | 42 C.; 1xSSC, 50% | |
| | | | formamide | |
| B | DNA:DNA | <50 | TB; 1xSSC | TB; 1xSSC |
| C | DNA:RNA | >50 | 67 C.; 1xSSC -or- | 67 C.; 0.3xSSC |
| | | | 45 C.; 1xSSC, 50% | |
| | | | formamide | |
| D | DNA:RNA | <50 | TD; 1xSSC | TD; 1xSSC |
| E | RNA:RNA | >50 | 70 C.; 1xSSC -or- | 70 C.; 0.3xSSC |
| | | | 50 C.; 1xSSC, 50% | |
| | | | formamide | |
| F | RNA:RNA | <50 | TF; 1xSSC | Tf; 1xSSC |
| G | DNA:DNA | >50 | 65 C.; 4xSSC -or- | 65 C.; 1xSSC |
| | | | 42 C.; 4xSSC, 50% | |
| | | | formamide | |
| H | DNA:DNA | <50 | TH; 4xSSC | TH; 4xSSC |
| I | DNA:RNA | >50 | 67 C.; 4xSSC -or- | 67 C.; 1xSSC |
| | | | 45 C.; 4xSSC, 50% | |
| | | | formamide | |
| J | DNA:RNA | <50 | TJ; 4Xssc | TJ; 4xSSC |
| K | RNA:RNA | >50 | 70 C.; 4xSSC -or- | 67 C.; 1xSSC |
| | | | 50 C.; 4xSSC, 50% | |
| | | | formamide | |
| L | RNA:RNA | <50 | TL; 2Xssc | TL; 2xSSC |
| M | DNA:DNA | >50 | 50 C.; 4xSSC -or- | 50 C.; 2xSSC |
| | | | 40 C.; 6xSSC, 50% | |
| | | | formamide | |
| N | DNA:DNA | <50 | TN; 6xSSC | TN; 6xSSC |
| O | DNA:RNA | >50 | 55 C.; 4xSSC -or- | 55 C.; 2xSSC |
| | | | 42 C.; 6xSSC, 50% | |
| | | | formamide | |
| P | DNA:RNA | <50 | TP; 6xSSC | TP; 6xSSC |
| Q | RNA:RNA | >50 | 60 C.; 4xSSC -or- | 60 C.; 2xSSC |
| | | | 45 C.; 6xSSC, 50% | |
| | | | formamide | |
| R | RNA:RNA | <50 | TR; 4xSSC | TR; 4xSSC |

The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarities.

bufferH: SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete.

TB through TR: The hybridization temperature for hybrids anticipated to be less than 50 base pairs in length should be about 5-10 C less than the melting temperature (Tm) of the hybrid, where Tm is determined according to the following equations. For hybrids less than 18 base pairs in length, Tm(C)=2(# of A+T bases)+4(# of G+C bases). For hybrids between 18 and 49 base pairs in length, Tm(C)=81.5+16.6(log 10[Na+])+0.41 (% G+C)-(600/N), where N is the number of bases in the hybrid, and [Na+] is the concentration of sodium ions in the hybridization buffer ([Na+] for 1×SSC=0.165 M).

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

In certain embodiments, modifications and changes are made in the structure of a polynucleotide of the present invention while retaining functional equivalency (such as immunogenicity, therapeutic benefit, binding affinity, etc) of the protein product encoded by the modified polypeptide. Such modifications and changes are fully contemplated by the present invention. For example, without limitation, certain amino acids can be substituted for other amino acids, including nonconserved and conserved substitution, in an amino sequence without appreciable loss of functionality/utility (e.g., immunogenicity, therapeutic benefit, etc.) and thus in the polynucleotide the corresponding codon encoding those amino acids can be changed accordingly, as would be understood by a person skilled in the art.

In fact, as it is the interactive capacity and nature of a polypeptide that defines that polypeptide's biological functional activity, a number of amino acid sequence substitutions are made in a polypeptide sequence, and thus its underlying nucleic acid coding sequence, and nevertheless obtain a polypeptide with like properties. The present invention contemplates any changes to the structure of the nucleic acid sequences encoding the subject polypeptides or proteins, wherein the polypeptide or protein retains its functionality or a biologically equivalent functionality. A person of ordinary skill in the art would be readily able to routinely modify the disclosed polypeptides and polynucleotides accordingly, based upon the guidance provided herein, while remaining consistent with the inventive concept and the purposes of the present invention (e.g., the use of the surrogate codons to enhance expression).

In making such changes, any techniques known to persons of skill in the art are utilized. For example, without intending to be limited thereto, the hydropathic index of amino acids can be considered, as described below with regard to the recombinant proteins and polypeptides of the present invention. The importance of the hydropathic amino acid index in conferring interactive biologic function on polypeptides is generally understood in the art. Kyte et al. 1982. J. Mol. Bio. 157:105-132.

According to further implementations of the invention, the polynucleotides comprise a polynucleotide library, such as a cDNA library. The preparation of such a library of polynucleotides is well known to persons of skill in the art. A person skilled in the art could readily prepare such a library in accordance with an embodiment of the present invention, using well known techniques and based upon the guidance provided herein. As described in further detail below, the polynucleotides of the invention are used in any suitable context, such as in vectors, immunogenic compositions, therapeutic compositions, recombinant cells and cell lines, assays, kits, tools, etc., as would be well understood by persons skilled in the art.

### Proteins and Polypeptides

The present invention also provides recombinant proteins or polypeptides encoded by the modified polynucleotides of the invention described herein. For example, in certain embodiments, a recombinant polypeptide or protein of the invention is a recombinant that is identical to the reference sequence of even numbered SEQ ID NOS: 2-6 or amino acid sequences encoded by any of odd numbered SEQ ID NOS:1-5 or any of SEQ ID NOS: 12-16 (which are sequences modified in accordance with the invention), that is, 100% identical, or it may include a number of amino acid alterations as compared to the reference sequence such that the percent identity is less than 100%. Such alterations include at least one amino acid deletion, substitution, including conservative and non-conservative substitution, or insertion. The alterations occur at the amino- or carboxy-terminal positions of the reference polypeptide sequence or anywhere between those terminal positions, interspersed either individually among the amino acids in the reference amino acid sequence or in one or more contiguous groups within the reference amino acid sequence.

Thus, the invention also provides proteins having sequence identity to an amino acid sequence of the invention, (e.g. even numbered SEQ ID NOS: 2-6 or proteins encoded by any of odd numbered SEQ ID NOS:1-5 or any of SEQ ID NOS:12-16). Depending on the particular sequence, the degree of sequence identity is greater than 60% (e.g., 60%, 70%, 80%, 85%, 90%, 94%, 95%, 97%, 98%, 99%, 99.9% or more). These homologous proteins include mutants and allelic variants.

In certain embodiments of the invention, the proteins or polypeptides (e.g., immunological portions and biological equivalents) generate antibodies. Specifically, the antibodies to the polypeptides protect from a challenge, such as intranasal. In further preferred embodiments, the polypeptides exhibit such protection for homologous strains and at least one heterologous strain. The polypeptide may be selected from even numbered SEQ ID NOS: 2-6 or amino acid sequences encoded by any of odd numbered SEQ ID NOS:1-5 or any of SEQ ID NOS: 12-16, or the polypeptide may be any immunological fragment or biological equivalent of the listed polypeptides. According to an embodiment, the polypeptide is selected from any of the even numbered SEQ ID NOS: 2-6 or amino acid sequences encoded by any of odd numbered SEQ ID NOS:1-5 or any of SEQ ID NOS: 12-16.

In certain embodiments, the invention relates to allelic or other variants of the polypeptides, which are biological equivalents. Suitable biological equivalents exhibit the ability to (1) elicit antibodies; (2) react with the surface of homologous strains and/or heterologous strains; (3) confer protection against a live challenge; and/or (4) prevent colonization.

Suitable biological equivalents have at least about 60% to about 100% similarity to one of the polypeptides specified herein (i.e., the even numbered SEQ ID NOS: 2-6 or amino acid sequences encoded by any of odd numbered SEQ ID NOS:1-5 or any of SEQ ID NOS: 12-16), provided the equivalent is capable of eliciting substantially the same immunogenic properties as one of the proteins of this invention.

Alternatively, the biological equivalents have substantially the same immunogenic properties of one of the proteins in the even numbered SEQ ID NOS: 2-6 or amino acid sequences encoded by any of odd numbered SEQ ID NOS:1-5 or any of SEQ ID NOS: 12-16. According to certain embodiments of the present invention, the biological equivalents have the same immunogenic properties as the even numbered SEQ ID NOS 2-6 or amino acid sequences encoded by any of odd numbered SEQ ID NOS:1-5 or any of SEQ ID NOS: 12-16.

The biological equivalents are obtained by generating variants and modifications to the proteins of this invention. These variants and modifications to the proteins are obtained by altering the amino acid sequences by insertion, deletion or substitution of one or more amino acids. The amino acid sequence is modified, for example by substitution in order to create a polypeptide having substantially the same or improved qualities. In a particular embodiment, a means of introducing alterations comprises making predetermined mutations of the nucleic acid sequence of the polypeptide by site-directed mutagenesis.

Modifications and changes can be made in the structure of a polypeptide of the present invention while retaining functional equivalency (such as immunogenicity, therapeutic benefit, binding affinity, etc). Such modifications and changes are fully contemplated by the present invention. For example, without limitation, certain amino acids can be substituted for other amino acids, including nonconserved and conserved substitution, in a sequence without appreciable loss of functionality/utility (e.g., immunogenicity, therapeutic benefit, etc.). The present invention contemplates any changes to the structure of the polypeptides herein, as well as the nucleic acid sequences encoding said polypeptides, wherein the polypeptide retains its functionality or a biologically equivalent functionality.

In making such changes, any techniques known to persons of skill in the art may be utilized. For example, without intending to be limited thereto, the hydropathic index, hydrophilicity, and the like, of amino acids are considered (Kyte et al. 1982. J. Mol. Bio. 157:105-132, U.S. Pat. No. 4,554,101).

Biological equivalents of a polypeptide are also prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. Such changes are desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is used, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the polypeptide sequence selected. An oligonucleotide primer bearing the desired mutated sequence is prepared (e.g., synthetically). This primer is then annealed to the single-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

The polypeptides of the invention include any protein or polypeptide comprising substantial sequence similarity and/or biological equivalence to a protein having an amino acid sequence of any of the proteins of the embodiments of the invention such as any of even numbered SEQ ID NOS 2-6 or proteins encoded by any of odd numbered SEQ ID NOS:1-5 and 12-16. In addition, the polypeptides of the invention are not limited to a particular source. Also, the polypeptides can be prepared recombinantly using any such technique in accordance with the purpose of the invention as described herein, as is well within the skill in the art, based upon the guidance provided herein, or in any other synthetic manner, as known in the art.

In certain embodiments, a polypeptide is cleaved into fragments for use in further structural or functional analysis, or in the generation of reagents such as related polypeptides and specific antibodies. This is accomplished by treating purified or unpurified polypeptides with a proteolytic enzyme (i.e., a proteinase) including, but not limited to, serine proteinases (e.g., chymotrypsin, trypsin, plasmin, elastase, thrombin, substilin) metal proteinases (e.g., carboxypeptidase A, carboxypeptidase B, leucine aminopeptidase, thermolysin, collagenase), thiol proteinases (e.g., papain, bromelain, Streptococcal proteinase, clostripain) and/or acid proteinases (e.g., pepsin, gastricsin, trypsinogen). Polypeptide fragments are also generated using chemical means such as treatment of the polypeptide with cyanogen bromide (CNBr), 2-nitro-5-thiocyanobenzoic acid, isobenzoic acid, BNPA-skatole, hydroxylamine or a dilute acid solution. In other embodiments, the polypeptide fragments of the invention are recombinantly expressed or prepared via peptide synthesis methods known in the art (Barany et al., 1997; U.S. Pat. No. 5,258,454).

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence. A typical variant of a polypeptide differs in amino acid sequence from another, reference polypeptide. Generally, differences are limited so that the sequences of the reference polypeptide and the variant are closely similar overall and, in many regions, identical (i.e., biologically equivalent). A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al 984), BLASTP, BLASTN, and FASTA (Altschul, S. F., et al, 1990). The BLASTX program is publicly available from NCBI and other sources (BLAST Manual, Altschul, S., et al., NCBI NLM NIH Bethesda, Md. 20894; Altschul, S., et al., 1990). The well known Smith Waterman algorithm may also be used to determine identity.

In certain embodiments, a polypeptide of the invention (e.g. any of the even numbered SEQ ID NOS:2-6) comprises modifications such as a mature processed form of a protein, lipidation, glycosylation, de-O-acylation, phosphorylation and the like.

In one particular embodiment, the polypeptides and nucleic acids encoding such polypeptides are used in immunogenic compositions for preventing or ameliorating infection.

The proteins of the invention, including the amino acid sequences of even numbered SEQ ID NOS: 2-6, their fragments, and analogs thereof, or cells expressing them, are also used as immunogens to produce antibodies immunospecific for the polypeptides of the invention.

### Antigens

In certain embodiments, an immunogenic composition, including proteins, polynucleotides and equivalents of the present invention, is administered as a sole active immunogen or alternatively, the composition includes other active immunogens and/or therapeutics, including other immunogenic polynucleotides, polypeptides, or immunologically-active proteins of one or more other microbial pathogens (e.g. virus, prion, bacterium, or fungus, without limitation) or capsular polysaccharide. The compositions may comprise one or more desired proteins, fragments or pharmaceutical compounds as desired for a chosen indication. In the same manner, the compositions of this invention which employ one or more nucleic acids in the composition may also include nucleic acids which encode the same diverse group of proteins, as noted above. In certain embodiments, a modified polynucleotide of the invention comprises a plasmid or a viral vector.

Any antigen, multi-antigen or multi-valent immunogenic composition is contemplated by the present invention. For example, the compositions of the present invention comprise a single protein, combinations of two or more proteins, one or more polysaccharides, a combination of one or more proteins, and one or more polysaccharides or any combination thereof. Persons of skill in the art would be readily able to formulate such immunogenic or therapeutic compositions.

The present invention also contemplates multi-immunization (e.g., a prime/boost regimen) or therapeutic regimens wherein any composition useful against a pathogen may be combined therein or therewith the compositions of the present invention. For example, without limitation, a mammalian subject is administered an immunogenic composition of the present invention and another composition, as part of a multi-drug regimen. Persons of skill in the art would be readily able to select compositions for use in conjunction with the immunogenic and/or therapeutic compositions of the present invention for the purposes of developing and implementing multi-drug regimens.

Specific embodiments of this invention relate to the use of one or more polypeptides of this invention, or nucleic acids encoding such, in a composition or as part of a treatment regimen for the prevention or amelioration of infection. One can combine the polypeptides or polynucleotides with any immunogenic composition for use against infection. One can also combine the polypeptides or polynucleotides with any other protein or polysaccharide-based immunogenic composition.

In certain embodiments, the polypeptides, fragments and equivalents are used as part of a conjugate immunogenic composition; wherein one or more proteins or polypeptides are conjugated to a carrier protein in order to generate a composition that has immunogenic properties against several serotypes and/or against several diseases. Alternatively, one of the polypeptides is used as a carrier protein for other immunogenic polypeptides.

The present invention also relates to a method of inducing immune responses in a mammal comprising the step of providing to said mammal an immunogenic composition of this invention. The immunogenic composition is a composition which is antigenic in the treated mammal such that an immunologically effective amount of the polypeptide(s) contained in such composition brings about the desired immune response against infection. Certain embodiments relate to a method for the treatment, including amelioration, or prevention of infection in a human comprising administering to a human an immunologically effective amount of the composition.

The phrase “immunologically effective amount,” as used herein, refers to the administration of that amount to a mammalian host (e.g., a human), either in a single dose or as part of a series of doses, sufficient to at least cause the immune system of the individual treated to generate a response that reduces the clinical impact of the bacterial or viral infection. This may range from a minimal decrease in bacterial or viral burden to prevention of the infection. Ideally, the treated individual will not exhibit the more serious clinical manifestations of the bacterial or viral infection. The dosage amount varies depending upon specific conditions of the individual. This amount is determined in routine trials or otherwise by means known to those skilled in the art.

The phrase “therapeutically effective amount”, as used herein, refers to the administration of that amount to a mammalian host (e.g., a human), either in a single dose or as part of a series of doses, sufficient to at least generate a response that reduces the impact of the pathogen on the host. The dosage amount can vary depending on the specific conditions of the host. The amount is determined through routine testing or otherwise as known to persons skilled in the art.

Another specific aspect of the present invention relates to using as the composition a vector or plasmid which expresses a protein of this invention, or an immunogenic or therapeutic portion thereof. Accordingly, a further aspect of the invention provides a method of inducing a desired response, e.g., immunogenic, in a mammal, which comprises providing to a mammal a vector or plasmid expressing at least one isolated polypeptide. The protein of the present invention is delivered to the mammal using a live, or live attenuted vectors. In certain embodiments, the virus is attenuated and comprises a modified polynucleotide encoding a bacterial protein, viral protein and the like, containing the genetic material necessary for the expression of the polypeptide or immunogenic portion as a foreign polypeptide.

### Viral and Non-Viral Vectors

The present invention also provides vectors comprising the polynucleotides of the present invention. According to various embodiments of the invention, vectors are used to transport recombinants of the invention to site of expression (e.g., transcription, translation/protein synthesis). Thus, the vectors are used in vivo or in vitro depending upon the desired objective. Any suitable vectors for accomplishing the objectives consistent with the inventive concept are contemplated by the present invention.

Viral vectors such as lentiviruses, retroviruses, herpes viruses, adenoviruses, adeno-associated viruses, vaccinia virus, baculovirus, and other recombinant viruses with desirable cellular tropism, are particularly useful for cellular assays in vitro and in vivo. Thus, a nucleic acid encoding a protein or immunogenic fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494, which is incorporated herein by reference in its entirety.

Viral vectors commonly used for in vivo or ex vivo targeting and therapy procedures include DNA vectors and RNA vectors. Methods for constructing and using viral vectors are known in the art (e.g., Miller and Rosman, BioTechniques, 1992, 7:980-990). In certain embodiments, the viral vectors are replication-defective, that is, they are unable to replicate autonomously in the target cell. In other embodiments, the viral vector is a live attenuated virus. In one particular embodiment, the replication defective virus is a minimal virus, i.e., it retains only the sequences of its genome which are necessary for encapsulating the genome to produce viral particles.

Various companies produce viral vectors commercially, including, but not limited to, Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors), incorporated by reference herein in its entirety.

Adenovirus vectors. Adenoviruses are eukaryotic DNA viruses that can be modified to efficiently deliver a nucleic acid of this invention to a variety of cell types. Various serotypes of adenovirus exist. In one particular embodiment, an adenovirus (Ad) is a type 2, type 4, type 5, or type 7 human adenoviruses (Ad 2, Ad 4, Ad 5 or Ad 7) or adenoviruses of animal origin (see PCT Publication No. WO 94/26914). Those adenoviruses of animal origin which can be used within the scope of the present invention include adenoviruses of canine, bovine, murine (e.g., Mav1, Beard et al., Virology, 1990, 75-81) bovine, porcine, avian, and simian (e.g., SAV) origin. In one embodiment, the adenovirus of animal origin is a canine adenovirus, such as a CAV2 adenovirus (e.g., Manhattan or A26/61 strain, ATCC VR-800). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques, which are well known to persons of ordinary skill in the art.

Adeno-associated viruses. The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retrovirus vectors. In another implementation of the present invention, the nucleic acid can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol, 1988, 62:1120; U.S. Pat. No. 5,124,263; European Publication Nos. EP 453 242 and EP178 220; Bernstein et al., Genet. Eng., 1985, 7:235; McCormick, BioTechnology, 1985, 3:689; PCT Publication No. WO 95/07358; and Kuo et al., Blood, 1993, 82:845, each of which is incorporated by reference in its entirety. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus”), MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences which may include a part of the gag gene (Bender et al., J. Virol, 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies transfection efficiency (see PCT Publication Nos. WO 95/22617, WO 95/26411, WO 96/39036 and WO 97/19182).

Lentivirus vectors. In another implementation of the present invention, lentiviral vectors are used as agents for the direct delivery and sustained expression of a transgene in several tissue types, including brain, retina, muscle, liver and blood. The vectors efficiently transduce dividing and nondividing cells in these tissues, and effect long-term expression of the gene of interest. For a review, see, Naldini, Curr. Opin. Biotechnol., 1998, 9:457-63; see also Zufferey, et al., J. Virol., 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 106 IU/mL for at least 3 to 4 days (Kafri, et al., J. Virol, 1999, 73: 576-584). The vector produced by the inducible cell line can be concentrated as needed for efficiently transducing non-dividing cells in vitro and in vivo.

In another implementation of the present invention, a modified polynucleotide of the invention is delivered via Mononegavirales. Viruses of the Order Mononegavirales are non-segmented, negative dtranded RNA viruses (e.g., described in U.S. Pat. No. 6,033,886, incorporated herein by reference)

In one particular embodiment, a modified polynucleotide of the invention is delivered via Vesicular Stomatitis Virus (VSV). Genetically modified VSV strains, attenuating VSV mutations and VSV rescue methods are well known in the art, e.g. see U.S. Pat. Nos. 6,033,886; 6,168,943; 6,596,529.

Non-viral vectors. In another implementation of the present invention, the vector can be introduced in vivo by lipofection, as “naked” DNA, or with other transfection facilitating agents (peptides, polymers, etc.). Synthetic cationic lipids are used to prepare liposomes for in vivo transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et al, supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

In certain embodiments, a polynucleotide modified for optimal expression in a mammalian host (i.e., comprising surrogate codons) is administered directly to the host as an immunogenic composition. The polynucleotide is introduced directly into the host either as “naked” DNA (U.S. Pat. No. 5,580,859) or formulated in compositions with agents which facilitate immunization, such as bupivicaine and other local anesthetics (U.S. Pat. No. 5,593,972) and cationic polyamines (U.S. Pat. No. 6,127,170).

In this polynucleotide immunization procedure, the polypeptides of the invention are expressed on a transient basis in vivo; no genetic material is inserted or integrated into the chromosomes of the host. This procedure is to be distinguished from gene therapy, where the goal is to insert or integrate the genetic material of interest into the chromosome. An assay is used to confirm that the polynucleotides administered by immunization do not give rise to a transformed phenotype in the host (U.S. Pat. No. 6,168,918).

It is also possible to introduce the vector in vivo as a naked DNA plasmid. Naked DNA vectors for vaccine purposes or gene therapy can be introduced into the desired host cells by methods known in the art, e.g., electroporation, microinjection, cell fusion, DEAE dextran, calcium phosphate precipitation, use of a gene gun, or use of a DNA vector transporter (e.g., Wu et al, J. Biol. Chem., 1992, 267:963-967; Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. More recently, a relatively low voltage, high efficiency in vivo DNA transfer technique, termed electrotransfer, has been described (Mir et al., C. P. Acad. Sci., 1988, 321:893; PCT Publication Nos. WO 99/01157; WO 99/01158; WO 99/01175). Accordingly, additional embodiments of the present invention relates to a method of inducing an immune response in a human comprising administering to said human an amount of a DNA molecule encoding a polypeptide of this invention, optionally with a transfection-facilitating agent, where said polypeptide, when expressed, retains the desired functionality and, when incorporated into an immunogenic composition and administered to a human, provides protection without inducing enhanced disease upon subsequent infection of the human with a pathogen. Transfection-facilitating agents are known in the art and include bupivicaine, and other local anesthetics (for examples see U.S. Pat. No. 5,739,118) and cationic polyamines (as published in International Patent Application WO 96/10038), which are hereby incorporated by reference.

According to an embodiment of the present invention, the IL-15 constructs as described herein are administered in a plasmid. According to an embodiment, the plasmid of the present invention comprises SEQ ID NOS: 18, 19, 20 or combinations thereof. The preparation of plasmids is well known in the art. A person of ordinary skill in the art could readily prepare a plasmid having the modified polynucleotide, such as the IL-15 constructs, for example, in accordance with the present invention, based upon the guidance provided herein. For example, the preparation of plasmids is described in U.S. Pat. No. 5,593,972, which is incorporated by reference in its entirety.

### Adjuvants

According to an embodiment of the present invention, the polynucleotides of the present invention may be used as adjuvants, for example, as adjuvants for vaccines, such as DNA and/or RNA vaccines. Techniques for the preparation of adjuvants, DNA vaccines and RNA vaccines are well known in the art. A person of skill in the art would readily be able to prepare an adjuvant, DNA vaccine and/or RNA vaccine and the like, using the embodiments of the present invention, based upon the guidance provided herein.

The present invention contemplates that the modified polynucleotides of the present invention may be used alone or in combination with other compounds or compositions for any desired effect. For example, the modified polynucleotides of the present invention may be administered in combination with a DNA and/or RNA vaccine or as part of the DNA and/or RNA vaccine (e.g., as part of a plasmid containing the DNA and/or RNA vaccine). The modified polyncleotides of the present invention may be administered separately but contemporaneously with the administration of the DNA and/or RNA vaccine, include administering during, before or after. Further, the polynucleotides of the present invention may be administered alone.

Exemplary DNA vaccines with which the present invention may be combined in any manner include, without limitation, nucleotides coding for the Plasmodium (malarial agent) proteins such as P. falciparum, P. vivax, P. malariae, and P. ovale CSP; SSP2(TRAP); Pfs16 (Sheba); LSA-1; LSA-2; LSA-3; STARP; MSA-1 (MSP-1, PMMSA, PSA, p185, p190); MSA-2 (MSP-2, Gymmsa, gp56, 38-45 kDa antigen); RESA (Pf155); EBA-175; AMA-1 (Pf83); SERA (p113, p126, SERP, Pf140); RAP-1; RAP-2; RhopH3; PfHRP-II; Pf55; Pf35; GBP (96-R); ABRA (p101); Exp-1 (CRA, Ag5.1); Aldolase; Duffy binding protein of P. vivax; Reticulocyte binding proteins; HSP70-1 (p75); Pfg25; Pfg28; Pfg48/45; and Pfg230. DNA and RNA vaccines also may comprise nucleotides coding for proteins associated with the GP or NP genes from the ebola virus; and the HPV6a L2, HPV6a E1, HPV6a E2, HPV6a E4, HPV6a E5, HPV6a E6, and HPV6a E7 proteins from the human Papillomavirus 6a (HPV6a). According to an embodiment, the DNA and RNA vaccines code for HIV proteins, including, but not limited to, the glycoproteins gp41, gp120, gp140, and gp160; and proteins encoded by the gag (the proteins p55, p39, p24, p17 and p15), env, rev, tat, nef, vpr, vpx, prot, and pol (the proteins p66/p51 and p31-34) genes found in HIV.

According to an embodiment of the present invention, the IL-15 constructs of the present invention (e.g., SEQ ID NOS:12-16) is used in combination with DNA and/or RNA vaccine. e.g, a DNA vaccine against HIV/AIDS. According to an embodiment, SEQ ID NO:14 is used (e.g., administered contemporaneously and/or combined in a plasmid or other vector or composition) in combination with a DNA vaccine against HIV/AIDS.

### Compositions

One aspect of the present invention provides compositions, such as immunogenic compositions and therapeutic compositions, etc., which comprise a modified polynucleotide of the present invention, a protein or polypeptide encoded by said recombinant polynucleotide, an antibody to said protein or polypeptide, or the like, including any combinations thereof. For example, compositions that have the ability to confer protection against a live challenge and/or prevent colonization are contemplated by the present invention.

The formulation of such compositions is well known to persons skilled in this field. Compositions of the invention, according to an embodiment, include a pharmaceutically acceptable carrier. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibody. The preparation and use of pharmaceutically acceptable carriers is well known in the art. Except insofar as any conventional media or agent is incompatible with the active ingredient, use thereof in the compositions of the present invention is contemplated.

An immunogenic composition of the invention is formulated to be compatible with its intended route of administration. Examples of routes of administration include parenteral (e.g., intravenous, intradermal, subcutaneous, intramuscular, intraperitoneal), mucosal (e.g., oral, rectal, intranasal, buccal, vaginal, respiratory) and transdermal (topical). Other modes of administration employ oral formulations, pulmonary formulations, suppositories, and transdermal applications, for example, without limitation. Oral formulations, for example, include such normally employed excipients as, for example, pharmaceutical grades of mannitol, lactose, starch, magnesium stearate, sodium saccharine, cellulose, magnesium carbonate, and the like, without limitation.

The present invention contemplates the use of embodiments of the invention as adjuvants or co-adjuvants, for example, as adjuvants to DNA or RNA vaccines/immunogenic composition. The immunogenic compositions of the invention can include one or more adjuvants, or be administered along with one or more adjuvants, including, but not limited to aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Mass.), described in U.S. Pat. No. 5,057,540, a pertussis toxin (PT), an E. coli heat-labile toxin (LT), particularly LT-K63, LT-R72, CT-S109, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, such as a histidine, in accordance with published International Patent Application number WO 00/18434).

Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine Interleukin-12 (IL-12) is another adjuvant which is described in U.S. Pat. No. 5,723,127. Other cytokines or lymphokines have been shown to have immune modulating activity, including, but not limited to, the interleukins 1-α, 1-β, 2, 4, 5, 6, 7, 8, 10, 13, 14, 15, 16, 17 and 18, the interferons-α, β and y, granulocyte colony stimulating factor, and the tumor necrosis factors α and β, and are suitable for use as adjuvants.

In certain embodiments, the proteins of this invention are used in a composition for oral administration which includes a mucosal adjuvant and used for the treatment or prevention of infection in a mammalian host (e.g., a human). The mucosal adjuvant can be a wild-type cholera toxin or; a derivative of a cholera holotoxin, wherein the A subunit is mutagenized or chemically modified. For a specific cholera toxin which may be particularly useful in preparing immunogenic compositions of this invention, see the mutant cholera holotoxin E29H, as disclosed in Published International Application WO 00/18434, which is hereby incorporated herein by reference in its entirety. These may be added to, or conjugated with, the polypeptides of this invention. The same techniques are applied to other molecules with mucosal adjuvant or delivery properties such as Escherichia coli heat labile toxin (LT). Other compounds with mucosal adjuvant or delivery activity may be used such as bile; polycations such as DEAE-dextran and polyornithine; detergents such as sodium dodecyl benzene sulphate; lipid-conjugated materials; antibiotics such as streptomycin; vitamin A; and other compounds that alter the structural or functional integrity of mucosal surfaces. Other mucosally active compounds include derivatives of microbial structures such as MDP; acridine and cimetidine. STIMULON™ QS-21, MPL, and IL-12, as described above, may also be used.

The compositions of this invention may be delivered in the form of ISCOMS (immune stimulating complexes), ISCOMS containing CTB, liposomes or encapsulated in compounds such as acrylates or poly(DL-lactide-co-glycoside) to form microspheres of a size suited to adsorption. The proteins of this invention may also be incorporated into oily emulsions.

Recombinant cells, recombinant cell lines, assays and kits that provide or use same and the like are also contemplated by the present invention. A person skilled in the art would readily understand how to prepare and use such embodiments of the present invention, based upon the guidance provided herein.

The present invention also relates to an antibody, which may either be a monoclonal or polyclonal antibody, specific for polypeptides as described above. Such antibodies may be produced by methods which are well known to those skilled in the art.

According to a further implementation of the present invention, a method is provided for diagnosing a condition in a mammal comprising: detecting the presence of immune complexes in the mammal or a tissue sample from said mammal, said mammal or tissue sample being contacted with an antibody composition comprising antibodies that immunospecifically bind with at least one polypeptide comprising the amino acid sequence of any of the even numbered SEQ ID NOS: 2-6; wherein the mammal or tissue sample is contacted with the antibody composition under conditions suitable for the formation of the immune complexes.

The description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art. A person skilled in the art would know, or be able to ascertain, using no more than routine experimentation, many equivalents to the specific embodiments of the invention described herein, based upon the guidance provided herein.

The following examples are included to demonstrate particular embodiments of the invention. However, those of skill in the art should, in view of the present disclosure, appreciate that many changes can be made in the specific embodiments which are disclosed and still obtain a like or similar result without departing from the spirit and scope of the invention. The following examples are offered by way of illustration and are not intended to limit the invention in any way.

### EXAMPLES

### Example 1

### Enhancement of HPV16 E7 expression

a. One example of a “modified” polynucleotide sequence demonstrating “enhanced” levels of protein expression is shown below in SEQ ID NO:1. The modified polynucleotide's sequence incorporates surrogate codons encoding the 98 amino acid human papillomavirus (HPV)16 E7 protein sequence (e.g., see HPV16 Accession No. K02718 in NCBI database).

The enhanced sequence of the polynucleotide in accordance with an embodiment of the invention is determined by selecting suitable surrogate codons. Surrogate codons were selected in order to alter the A and T (or A and U in the case of RNA) content of the naturally-occurring (wild-type) gene. The surrogate codons are those that encode the amino acids alanine, arginine, glutamic acid, glycine, isoleucine, leucine, proline, serine, threonine, and valine. Accordingly, the modified nucleic acid sequence had surrogate codons for each of these amino acids throughout the sequence. For the remaining 11 amino acids, no alterations were made, thereby leaving the corresponding naturally-occurring codons in place.

The modified sequence may be determined manually or by computer-assisted methods. As such, the information technology, including hardware, software, algorithms, arrays, databases and the like, directed to the determination of the modified sequences of the present invention are contemplated herein.

| SEQ ID NO:1 (polynucleotide) and SEQ ID NO:2 (protein) | |
|---|---|
| 1 | ATGCATGGGGATACGCCTACGCTCCATGAATATATGCTCGATCTCCAACCTGA |
| 1 | M H G D T P T L H E Y M L D L Q P E |
| 54 | GACGACGGATCTCTACTGTTATGAGCAACTCAATGACAGCTCCGAGGAGGAGG |
| 18 | T T D L Y C Y E Q L N D S S E E E |
| 107 | ATGAAATTGATGGGCCTGCGGGGCAAGCGGAACCTGACCGGGCCCATTACAAT |
| 36 | D E I D G P A G Q A E P D R A H Y N |
| 160 | ATTGTCACCTTTTGTTGCAAGTGTGACTCCACGCTCCGGCTCTGCGTCCAAAG |
| 54 | I V T F C C K C D S T L R L C V Q S |
| 213 | CACGCACGTCGACATTCGGACGCTCGAAGACCTGCTCATGGGCACGCTCGGGA |
| 71 | T H V D I R T L E D L L M G T L G |
| 266 | TTGTGTGCCCCATCTGTTCCCAGAAACCTTAATAG |
| 89 | I V C P I C S Q K P |

Referring to SEQ ID NO:1 above, the recombinant nucleotide sequence of HPV16 E7 (Accession No. K02718) incorporates surrogate codons but retains the capacity to encode the wild type E7 protein.

b. The nucleic acid sequence of SEQ ID NO:1 was assembled from oligonucleotides that were 100 nucleotides in length and corresponding in polarity to the positive (sense) strand sequence shown above. A person of skill in the art would readily be able to select suitable oligonucleotides depending upon the desired sequence in accordance with the present invention. Suitable oligonucleotides are available from a variety of commercial vendors, such as Invitrogen™ (Carlsbad, Calif.).

“Bridge” oligos 50 nucleotides in length and antisense in polarity were designed to straddle the joints at the ends of each sense 100-mer oligo. This strategy facilitated the hybridization of 25 nucleotides at the ends of each 100-mer targeted for ligation. A heat stable ligase (Ampligase, Epicentre, Wis.) was used at 68° C. to ligate the 100-mer sense oligos together. The entire open reading frame (for HPV16 E7, approximately 300 nucleotides) was then PCR amplified using oligos corresponding to the 5′ and 3′ boundaries of the ORF. The fidelity of the intended final ORF was verified by sequencing reactions.

This HPV16E7 gene containing surrogate codons was tested for expression levels by Western blot (data not shown). Rhabdomyosarcoma (RD) cells (American Type Culture Collection, Manassas, Va. ATCC# CCL136) were transfected with the indicated plasmid DNA expression vectors. All HPV16 E7 genes were cloned into pcDNA3.1 (Invitrogen, Carlsbad, Calif.). While a variety of different transfecting agents could be utilized, the experiments listed herein were performed using Lipofectamine (invitrogen) according to manufacturer's instructions. Total cell lysates were harvested 48 hours after transfection in SDS-sample buffer containing 1% SDS and 2-mercaptoethanol. Equivalent amounts of each transfectant lysate were loaded and electrophoresed on 4-20% tris-glycine gradient SDS-polyacrylamide gels. HPV16 E7 protein was detected by an E7-specific monoclonal antibody (Zymed Laboratories, San Francisco, Calif.).

The expression levels of the surrogate codon modified HPV16 E7 gene (SEQ ID NO:1) were markedly enhanced compared to the expression levels of the wild type HPV16 E7 gene. The expression levels of the surrogate codon modified HPV16E7 was comparable to the expression level of the “preferred” codon modified HPV16E7 (data not shown).

### Example 2

### Enhancement of HIV-1 Gag p37 Expression

A second example demonstrating the unexpected results of using “surrogate” codons in lieu of wild-type codons in a nucleic acid sequence was found for the HIV-1 gag gene, specifically the p37 component of the full-length p55 protein.

a. The amino acid sequence of the HXB2 strain of HIV-1 (NCBI Accession No. K03455) was selected as a representative HIV-1 gag gene.

| SEQ ID NO:3 (polynucleotide) and SEQ ID NO:4 (protein) | |
|---|---|
| 1 | ATGGGGGCGCGGGCGTCCGTCCTCTCCGGGGGGGAGCTCGATCGGTGGGAGAAA |
| 1 | M G A R A S V L S G G E L D R W E K |
| 55 | ATTCGGCTCCGGCCGGGGGGGAAGAAAAAATATAAACTCAAACATATTGTCTGG |
| 19 | I R L R P G G K K K Y K L K H I V W |
| 109 | GCGTCCCGGGAGCTCGAGCGGTTCGCGGTCAATCCGGGGCTGCTCGAGACGTCC |
| 37 | A S R E L E R F A V N P G L L E T S |
| 163 | GAGGGCTGTCGGCAAATTCTCGGGCAGCTCCAACCGTCCCTCCAGACGGGGTCC |
| 55 | E G C R Q I L G Q L Q P S L Q T G S |
| 217 | GAGGAGCTCCGGTCCCTCTATAATACGGTCGCGACGCTCTATTGTGTCCATCAA |
| 73 | E E L R S L Y N T V A T L Y C V H Q |
| 271 | CGGATTGAGATTAAAGACACGAAGGAGGCGCTCGACAAGATTGAGGAGGAGCAA |
| 91 | R I E I K D T K E A L D K I E E E Q |
| 325 | AACAAATCCAAGAAAAAAGCGCAGCAAGCGGCGGCGGACACGGGGCACTCCAAT |
| 109 | N K S K K K A Q Q A A A D T G H S N |
| 379 | CAGGTCTCCCAAAATTACCCGATTGTCCAGAACATTCAGGGGCAAATGGTCCAT |
| 127 | Q V S Q N Y P I V Q N I Q G Q M V H |
| 433 | CAGGCGATTTCCCCGCGGACGCTCAATGCGTGGGTCAAAGTCGTCGAGGAGAAG |
| 145 | Q A I S P R T L N A W V K V V E E K |
| 487 | GCGTTCTCCCCGGAGGTCATTCCGATGTTTTCAGCGCTCTCCGAGGGGGCGACG |
| 163 | A F S P E V I P M F S A L S E G A T |
| 541 | CCGCAAGATCTCAACACGATGCTCAACACGGTCGGGGGGCATCAAGCGGCGATG |
| 181 | P Q D L N T M L N T V G G H Q A A M |
| 595 | CAAATGCTCAAAGAGACGATTAATGAGGAGGCGGCGGAGTGGGATCGGGTCCAT |
| 199 | Q M L K E T I N E E A A E W D R V H |
| 649 | CCGGTCCATGCGGGGCCGATTGCGCCGGGGCAGATGCGGGAGCCGCGGGGGTCC |
| 217 | P V H A G P I A P G Q M R E P R G S |
| 703 | GACATTGCGGGGACGACGTCCACGCTCCAGGAGCAAATTGGGTGGATGACGAAT |
| 235 | D I A G T T S T L Q E Q I G W M T N |
| 757 | AATCCGCCGATTCCGGTCGGGGAGATTTATAAACGGTGGATTATTCTCGGGCTC |
| 253 | N P P I P V G E I Y K R W I I L G L |
| 811 | AATAAAATTGTCCGGATGTATTCCCCGACGTCCATTCTCGACATTCGGCAAGGG |
| 271 | N K I V R M Y S P T S I L D I R Q G |
| 865 | CCCAAGGAGCCGTTTCGGGACTATGTAGACCGGTTCTATAAAACGCTCCGGGCG |
| 289 | P K E P F R D Y V D R F Y K T L R A |
| 919 | GAGCAAGCGTCCCAGGAGGTCAAAAATTGGATGACGGAGACGCTCCTCGTCCAA |
| 307 | E Q A S Q E V K N W M T E T L L V Q |
| 973 | AATGCGAACCCGGATTGTAAGACGATTCTCAAAGCGCTCGGGCCGGCGGCTACG |
| 325 | N A N P D C K T I L K A L G P A A T |
| 1027 | CTCGAGGAGATGATGACGGCGTGTCAGGGGGTCGGGGGGCCGGGGCATAAGGCG |
| 343 | L E E M M T A C Q G V G G P G H K A |
| 1081 | CGGGTCCTCTAA |
| 361 | R V L |

Referring to SEQ ID NO:3, an altered nucleotide sequence of the HXB2 strain of HIV-1 gag gene (Accession No. K03455) incorporating surrogate codons but retaining the capacity to encode the 363 amino acid wild type p37 component of the gag protein, was constructed.

The HIV-1 gag p37 gene incorporating surrogate codons was assembled by a different method than that used for the HPV16 E7 (Example 1). This gene was assembled using a series of 100-mer sense and antisense oligos containing overlapping 25 nucleotides of sequence as illustrated below.

^{P}ATG . . . 3′ ^{P} . . . 3′
3′. . . ^{P} etc.

Each 100 mer was phosphorylated (^{P}) on the 5′ end to facilitate downstream ligation. For reference, the 5′ end of the gag gene, containing the initiation codon ATG, is depicted (sense oligo); an antisense oligo beneath it was designed to contain complementary sequence of 25 nucleotides to facilitate hybridization and subsequent fill in by a DNA polymerase (Pfx Turbo, Invitrogen). This staggered, overlapping arrangement was performed to assemble the entire ˜1.1 kb gag gene encoding p37. The double stranded but “nicked” assembled gene was then ligated using a heat stable ligase (Ampligase).

PCR oligos representing the 5′ and 3′ most regions of the p37 ORF were then used to amplify the entire gene, which was subsequently cloned into the vector and sequenced to confirm the fidelity in assembly of the predicted sequence.

The expression levels of a plasmid DNA construct containing the altered/“surrogate” gag p37 gene shown above were tested by transfection in Cos7 cells (ATCC CRL 1651). The levels of gag present in the supernatant 48 hours post infection was quantified with an ELISA assay using a commercially available kit (Coulter p24 kit, Beckman Coulter catalog #PN6604535). The plasmid construct set forth in SEQ ID NO:7 was used for transfection of the wild-type gag p37. The plasmid construct set forth in SEQ ID NO:8 was used for transfection of the recombinant gag gene (modified in accordance with an embodiment of the present invention).

| SEQ ID NO:7 | |
|---|---|
| aaatgggggc gctgaggtct gcctcgtgaa gaaggtgttg ctgactcata ccaggcctga | 60 |
| atcgccccat catccagcca gaaagtgagg gagccacggt tgatgagagc tttgttgtag | 120 |
| gtggaccagt tggtgatttt gaacttttgc tttgccacgg aacggtctgc gttgtcggga | 180 |
| agatgcgtga tctgatcctt caactcagca aaagttcgat ttattcaaca aagccgccgt | 240 |
| cccgtcaagt cagcgtaatg ctctgccagt gttacaacca attaaccaat tctgcgttca | 300 |
| aaatggtatg cgttttgaca catccactat atatccgtgt cgttctgtcc actcctgaat | 360 |
| cccattccag aaattctcta gcgattccag aagtttctca gagtcggaaa gttgaccaga | 420 |
| cattacgaac tggcacagat ggtcataacc tgaaggaaga tctgattgct taactgcttc | 480 |
| agttaagacc gacgcgctcg tcgtataaca gatgcgatga tgcagaccaa tcaacatggc | 540 |
| acctgccatt gctacctgta cagtcaagga tggtagaaat gttgtcggtc cttgcacacg | 600 |
| aatattacgc catttgcctg catattcaaa cagctcttct acgataaggg cacaaatcgc | 660 |
| atcgtggaac gtttgggctt ctaccgattt agcagtttga tacactttct ctaagtatcc | 720 |
| acctgaatca taaatcggca aaatagagaa aaattgacca tgtgtaagcg gccaatctga | 780 |
| ttccacctga gatgcataat ctagtagaat ctcttcgcta tcaaaattca cttccacctt | 840 |
| ccactcaccg gttgtccatt catggctgaa ctctgcttcc tctgttgaca tgacacacat | 900 |
| catctcaata tccgaatacg gaccatcagt ctgacgacca agagagccat aaacaccaat | 960 |
| agccttaaca tcatccccat atttatccaa tattcgttcc ttaatttcat gaacaatctt | 1020 |
| cattctttct tctctagtca ttattattgg tccgttcata acaccccttg tattactgtt | 1080 |
| tatgtaagca gacagtttta ttgttcatga tgatatattt ttatcttgtg caatgtaaca | 1140 |
| tcagagattt tgagacacaa cgtggctttc cccggcccat gaccaaaatc ccttaacgtg | 1200 |
| agttttcgtt ccactgagcg tcagaccccg tagaaaagat caaaggatct tcttgagatc | 1260 |
| ctttttttct gcgcgtaatc tgctgcttgc aaacaaaaaa accaccgcta ccagcggtgg | 1320 |
| tttgtttgcc ggatcaagag ctaccaactc tttttccgaa ggtaactggc ttcagcagag | 1380 |
| cgcagatacc aaatactgtc cttctagtgt agccgtagtt aggccaccac ttcaagaact | 1440 |
| ctgtagcacc gcctacatac ctcgctctgc taatcctgtt accagtggct gctgccagtg | 1500 |
| gcgataagtc gtgtcttacc gggttggact caagacgata gttaccggat aaggcgcagc | 1560 |
| ggtcgggctg aacggggggt tcgtgcacac agcccagctt ggagcgaacg acctacaccg | 1620 |
| aactgagata cctacagcgt gagctatgag aaagcgccac gcttcccgaa gggagaaagg | 1680 |
| cggacaggta tccggtaagc ggcagggtcg gaacaggaga gcgcacgagg gagcttccag | 1740 |
| ggggaaacgc ctggtatctt tatagtcctg tcgggtttcg ccacctctga cttgagcgtc | 1800 |
| gatttttgtg atgctcgtca ggggggcgga gcctatggaa aaacgccagc aacgcggcct | 1860 |
| ttttacggtt cctggccttt tgctggcctt ttgctcacat gttctttcct gcgttatccc | 1920 |
| ctgattctgt ggataaccgt attaccgcct ttgagtgagc tgataccgct cgccgcagcc | 1980 |
| gaacgaccga gcgcagcgag tcagtgagcg aggaagcgga agagcgcctg atgcggtatt | 2040 |
| ttctccttac gcatctgtgc ggtatttcac accgcatatg gtgcactctc agtacaatct | 2100 |
| gctctgatgc cgcatagtta agccagtatc tgctccctgc ttgtgtgttg gaggtcgctg | 2160 |
| agtagtgcgc gagcaaaatt taagctacaa caaggcaagg cttgaccgac aattgcatga | 2220 |
| agaatctgct tagggttagg cgttttgcgc tgcttcgcga tgtacgggcc agatatagcc | 2280 |
| gcggcatcga tgatatccat tgcatacgtt gtatctatat cataatatgt acatttatat | 2340 |
| tggctcatgt ccaatatgac cgccatgttg acattgatta ttgactagtt attaatagta | 2400 |
| atcaattacg gggtcattag ttcatagccc atatatggag ttccgcgtta cataacttac | 2460 |
| ggtaaatggc ccgcctggct gaccgcccaa cgacccccgc ccattgacgt caataatgac | 2520 |
| gtatgttccc atagtaacgc caatagggac tttccattga cgtcaatggg tggagtattt | 2580 |
| acggtaaact gcccacttgg cagtacatca agtgtatcat atgccaagtc cgccccctat | 2640 |
| tgacgtcaat gacggtaaat ggcccgcctg gcattatgcc cagtacatga ccttacggga | 2700 |
| ctttcctact tggcagtaca tctacgtatt agtcatcgct attaccatgg tgatgcggtt | 2760 |
| ttggcagtac atcaatgggc gtggatagcg gtttgactca cggggatttc caagtctcca | 2820 |
| ccccattgac gtcaatggga gtttgttttg gcaccaaaat caacgggact ttccaaaatg | 2880 |
| tcgtaacaac tccgccccat tgacgcaaat gggcggtagg cgtgtacggt gggaggtcta | 2940 |
| tataagcaga gctcgtttag tgaaccgtca gatcgcctgg agacgccatc cacgctgttt | 3000 |
| tgacctccat agaagacacc gggaccgatc cagcctccgc gggcgcgcgt cgacagagag | 3060 |
| atgggtgcga gagcgtcagt attaagcggg ggagaattag atcgatggga aaaaattcgg | 3120 |
| ttaaggccag ggggaaagaa aaaatataaa ttaaaacata tagtatgggc aagcagggag | 3180 |
| ctagaacgat tcgcagttaa tcctggcctg ttagaaacat cagaaggctg tagacaaata | 3240 |
| ctgggacagc tacaaccatc ccttcagaca ggatcagaag aacttagatc attatataat | 3300 |
| acagtagcaa ccctctattg tgtgcatcaa aggatagaga taaaagacac caaggaagct | 3360 |
| ttagacaaga tagaggaaga gcaaaacaaa agtaagaaaa aagcacagca agcagcagct | 3420 |
| gacacaggac acagcaatca ggtcagccaa aattacccta tagtgcagaa catccagggg | 3480 |
| caaatggtac atcaggccat atcacctaga actttaaatg catgggtaaa agtagtagaa | 3540 |
| gagaaggctt tcagcccaga agtgataccc atgttttcag cattatcaga aggagccacc | 3600 |
| ccacaagatt taaacaccat gctaaacaca gtggggggac atcaagcagc catgcaaatg | 3660 |
| ttaaaagaga ccatcaatga ggaagctgca gaatgggata gagtgcatcc agtgcatgca | 3720 |
| gggcctattg caccaggcca gatgagagaa ccaaggggaa gtgacatagc aggaactact | 3780 |
| agtacccttc aggaacaaat aggatggatg acaaataatc cacctatccc agtaggagaa | 3840 |
| atttataaaa gatggataat cctgggatta aataaaatag taagaatgta tagccctacc | 3900 |
| agcattctgg acataagaca aggaccaaaa gaacccttta gagactatgt agaccggttc | 3960 |
| tataaaactc taagagccga gcaagcttca caggaggtaa aaaattggat gacagaaacc | 4020 |
| ttgttggtcc aaaatgcgaa cccagattgt aagactattt taaaagcatt gggaccagcg | 4080 |
| gctacactag aagaaatgat gacagcatgt cagggagtag gaggacccgg ccataaggca | 4140 |
| agagttttgt aggtttaaac taagccgaat tctgcagatc gcgccgagct cgctgatcag | 4200 |
| cctcgactgt gccttctagt tgccagccat ctgttgtttg cccctccccc gtgccttcct | 4260 |
| tgaccctgga aggtgccact cccactgtcc tttcctaata aaatgaggaa attgcatcgc | 4320 |
| attgtctgag taggtgtcat tctattctgg ggggtggggt ggggcaggac agcaaggggg | 4380 |
| aggattggga agacaatagc aggcatgctg gggaattt | 4418 |
| SEQ ID NO:8 | |
| aaatgggggc gctgaggtct gcctcgtgaa gaaggtgttg ctgactcata ccaggcctga | 60 |
| atcgccccat catccagcca gaaagtgagg gagccacggt tgatgagagc tttgttgtag | 120 |
| gtggaccagt tggtgatttt gaacttttgc tttgccacgg aacggtctgc gttgtcggga | 180 |
| agatgcgtga tctgatcctt caactcagca aaagttcgat ttattcaaca aagccgccgt | 240 |
| cccgtcaagt cagcgtaatg ctctgccagt gttacaacca attaaccaat tctgcgttca | 300 |
| aaatggtatg cgttttgaca catccactat atatccgtgt cgttctgtcc actcctgaat | 360 |
| cccattccag aaattctcta gcgattccag aagtttctca gagtcggaaa gttgaccaga | 420 |
| cattacgaac tggcacagat ggtcataacc tgaaggaaga tctgattgct taactgcttc | 480 |
| agttaagacc gacgcgctcg tcgtataaca gatgcgatga tgcagaccaa tcaacatggc | 540 |
| acctgccatt gctacctgta cagtcaagga tggtagaaat gttgtcggtc cttgcacacg | 600 |
| aatattacgc catttgcctg catattcaaa cagctcttct acgataaggg cacaaatcgc | 660 |
| atcgtggaac gtttgggctt ctaccgattt agcagtttga tacactttct ctaagtatcc | 720 |
| acctgaatca taaatcggca aaatagagaa aaattgacca tgtgtaagcg gccaatctga | 780 |
| ttccacctga gatgcataat ctagtagaat ctcttcgcta tcaaaattca cttccacctt | 840 |
| ccactcaccg gttgtccatt catggctgaa ctctgcttcc tctgttgaca tgacacacat | 900 |
| catctcaata tccgaatacg gaccatcagt ctgacgacca agagagccat aaacaccaat | 960 |
| agccttaaca tcatccccat atttatccaa tattcgttcc ttaatttcat gaacaatctt | 1020 |
| cattctttct tctctagtca ttattattgg tccgttcata acaccccttg tattactgtt | 1080 |
| tatgtaagca gacagtttta ttgttcatga tgatatattt ttatcttgtg caatgtaaca | 1140 |
| tcagagattt tgagacacaa cgtggctttc cccggcccat gaccaaaatc ccttaacgtg | 1200 |
| agttttcgtt ccactgagcg tcagaccccg tagaaaagat caaaggatct tcttgagatc | 1260 |
| ctttttttct gcgcgtaatc tgctgcttgc aaacaaaaaa accaccgcta ccagcggtgg | 1320 |
| tttgtttgcc ggatcaagag ctaccaactc tttttccgaa ggtaactggc ttcagcagag | 1380 |
| cgcagatacc aaatactgtc cttctagtgt agccgtagtt aggccaccac ttcaagaact | 1440 |
| ctgtagcacc gcctacatac ctcgctctgc taatcctgtt accagtggct gctgccagtg | 1500 |
| gcgataagtc gtgtcttacc gggttggact caagacgata gttaccggat aaggcgcagc | 1560 |
| ggtcgggctg aacggggggt tcgtgcacac agcccagctt ggagcgaacg acctacaccg | 1620 |
| aactgagata cctacagcgt gagctatgag aaagcgccac gcttcccgaa gggagaaagg | 1680 |
| cggacaggta tccggtaagc ggcagggtcg gaacaggaga gcgcacgagg gagcttccag | 1740 |
| ggggaaacgc ctggtatctt tatagtcctg tcgggtttcg ccacctctga cttgagcgtc | 1800 |
| gatttttgtg atgctcgtca ggggggcgga gcctatggaa aaacgccagc aacgcggcct | 1860 |
| ttttacggtt cctggccttt tgctggcctt ttgctcacat gttctttcct gcgttatccc | 1920 |
| ctgattctgt ggataaccgt attaccgcct ttgagtgagc tgataccgct cgccgcagcc | 1980 |
| gaacgaccga gcgcagcgag tcagtgagcg aggaagcgga agagcgcctg atgcggtatt | 2040 |
| ttctccttac gcatctgtgc ggtatttcac accgcatatg gtgcactctc agtacaatct | 2100 |
| gctctgatgc cgcatagtta agccagtatc tgctccctgc ttgtgtgttg gaggtcgctg | 2160 |
| agtagtgcgc gagcaaaatt taagctacaa caaggcaagg cttgaccgac aattgcatga | 2220 |
| agaatctgct tagggttagg cgttttgcgc tgcttcgcga tgtacgggcc agatatagcc | 2280 |
| gcggcatcga tgatatccat tgcatacgtt gtatctatat cataatatgt acatttatat | 2340 |
| tggctcatgt ccaatatgac cgccatgttg acattgatta ttgactagtt attaatagta | 2400 |
| atcaattacg gggtcattag ttcatagccc atatatggag ttccgcgtta cataacttac | 2460 |
| ggtaaatggc ccgcctggct gaccgcccaa cgacccccgc ccattgacgt caataatgac | 2520 |
| gtatgttccc atagtaacgc caatagggac tttccattga cgtcaatggg tggagtattt | 2580 |
| acggtaaact gcccacttgg cagtacatca agtgtatcat atgccaagtc cgccccctat | 2640 |
| tgacgtcaat gacggtaaat ggcccgcctg gcattatgcc cagtacatga ccttacggga | 2700 |
| ctttcctact tggcagtaca tctacgtatt agtcatcgct attaccatgg tgatgcggtt | 2760 |
| ttggcagtac atcaatgggc gtggatagcg gtttgactca cggggatttc caagtctcca | 2820 |
| ccccattgac gtcaatggga gtttgttttg gcaccaaaat caacgggact ttccaaaatg | 2880 |
| tcgtaacaac tccgccccat tgacgcaaat gggcggtagg cgtgtacggt gggaggtcta | 2940 |
| tataagcaga gctcgtttag tgaaccgtca gatcgcctgg agacgccatc cacgctgttt | 3000 |
| tgacctccat agaagacacc gggaccgatc cagcctccgc gggcgcgcgt cgacgccacc | 3060 |
| atgggggcgc gggcgtccgt cctctccggg ggggagctcg atcggtggga gaaaattcgg | 3120 |
| ctccggccgg gggggaagaa aaaatataaa ctcaaacata ttgtctgggc gtcccgggag | 3180 |
| ctcgagcggt tcgcggtcaa tccggggctg ctcgagacgt ccgagggctg tgcgcaaatt | 3240 |
| ctcgggcagc tccaaccgtc cctccagacg gggtccgagg agctccggtc cctctataat | 3300 |
| acggtcgcga cgctctattg tgtccatcaa cggattgaga ttaaagacac gaaggaggcg | 3360 |
| ctcgacaaga ttgaggagga gcaaaacaaa tccaagaaaa aagcgcagca agcggcggcg | 3420 |
| gacacggggc actccaatca ggtctcccaa aattacccga ttgtccagaa cattcagggg | 3480 |
| caaatggtcc atcaggcgat ttccccgcgg acgctcaatg cgtgggtcaa agtcgtcgag | 3540 |
| gagaaggcgt tctccccgga ggtcattccg atgttttcag cgctctccga gggggcgacg | 3600 |
| ccgcaagatc tcaacacgat gctcaacacg gtcggggggc atcaagcggc gatgcaaatg | 3660 |
| ctcaaagaga cgattaatga ggaggcggcg gagtgggatc gggtccatcc ggtccatgcg | 3720 |
| gggccgattg cgccggggca gatgcgggag ccgcgggggt ccgacattgc ggggacgacg | 3780 |
| tccacgctcc aggagcaaat tgggtggatg acgaataatc cgccgattcc ggtcggggag | 3840 |
| atttataaac ggtggattat tctcgggctc aataaaattg tccggatgta ttccccgacg | 3900 |
| tccattctcg acattcggca agggccgaag gagccgtttc gggactatgt agaccggttc | 3960 |
| tataaaacgc tccgggcgga gcaagcgtcc caggaggtca aaaattggat gacggagacg | 4020 |
| ctcctcgtcc aaaatgcgaa cccggattgt aagacgattc tcaaagcgct cgggccggcg | 4080 |
| gctacgctcg aggagatgat gacggcgtgt cagggggtcg gggggccggg gcataaggcg | 4140 |
| cgggtcctct aatgaggcgc gccgagctcg ctgatcagcc tcgactgtgc cttctagttg | 4200 |
| ccagccatct gttgtttgcc cctcccccgt gccttccttg accctggaag gtgccactcc | 4260 |
| cactgtcctt tcctaataaa atgaggaaat tgcatcgcat tgtctgagta ggtgtcattc | 4320 |
| tattctgggg ggtggggtgg ggcaggacag caagggggag gattgggaag acaatagcag | 4380 |
| gcatgctggg gaattt | 4396 |

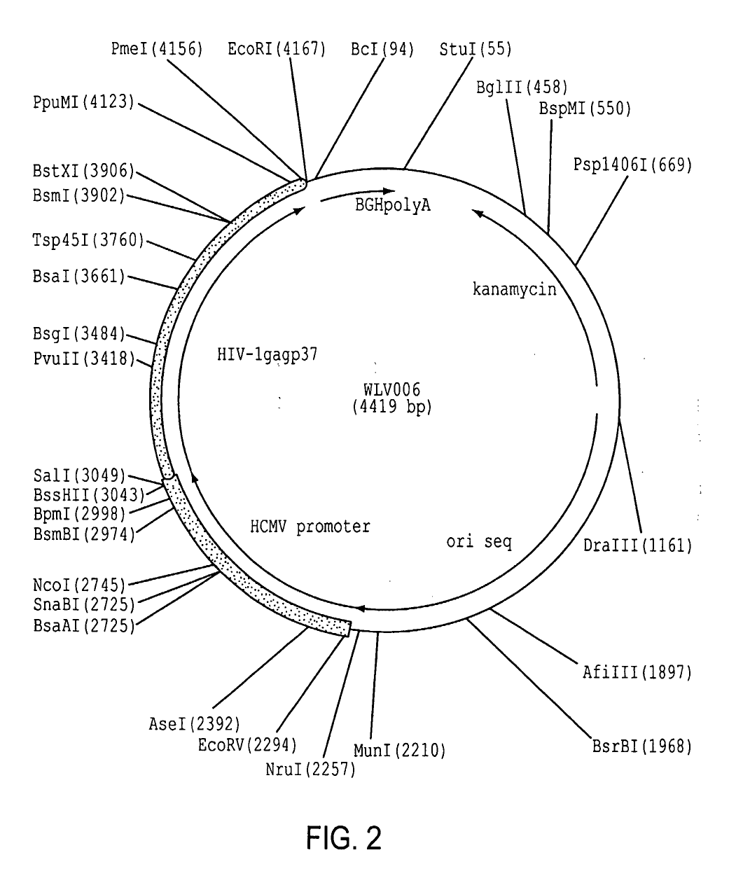

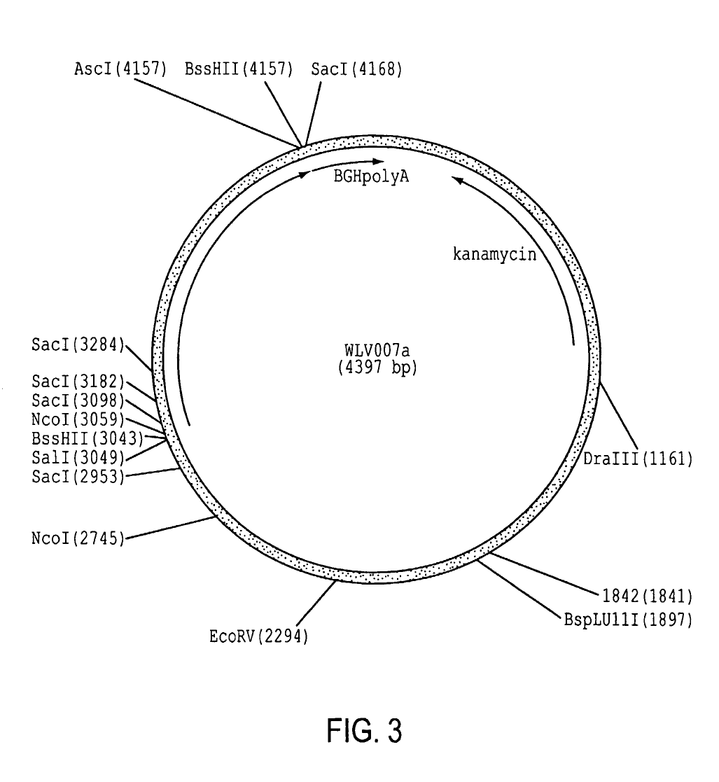

A plasmid map of the plasmid construct set forth in SEQ ID NO:7 is provided as FIG. 2 and a plasmid map of the plasmid construct as set forth in SEQ ID NO:8 is provided as FIG. 3.

The results of two experiments to compare the levels of gag expression of the wild-type to the modified gene are provided in Table III.

TABLE III

Experiment 1:
Expression from wild-type gag (plasmid construct of SEQ ID
NO: 7) = 8 ng/ml
Expression from modified gag (plasmid construct of SEQ ID
NO: 8) = 88 ng/ml
Experiment 2:
Expression from wild-type gag (SEQ ID NO: 7) = 0.6 ng/ml
Expression from modified gag (SEQ ID NO: 8) = 10 ng/ml

As indicated by the experimental results provided in Table III, the modified polynucleotide prepared in accordance with an embodiment of the present invention provided at least a ten fold increase in expression over its corresponding wild-type polynucleotide.

### Example 3

### Enhancement of Expression of HIV-1 gp160 Envelope Primary Isolate 6101

a. A third example illustrating the unexpected benefits of using “surrogate” codons in lieu of wild-type codons in a nucleic acid sequence was found for an HIV-1 gp160 envelope gene derived from a primary isolate 6101. The sequences (SEQ ID NO:5, the modified polynucleotide, and SEQ ID NO:6, the protein) are provided below.

| SEQ ID NO:5 (polypeptide) and SEQ ID NO:6 (protein) | |
|---|---|
| 1 | ATGCGGGCGAAGGAGATGCGGAAGTCCTGTCAGCACCTCCGGAAATGGGGGATTCTCCTCTTTGGGGTCCTCATGATTTGT |
| 1 | M R A K E M R K S C Q K L R K W G I L L F G V L M I C |
| 82 | TCCGCGGAGGAGAAGCTCTGGGTCACGGTCTATTATGGGGTCCCGGTCTGGAAAGAGGCGACGACGACGCTCTTTTGTGCG |
| 28 | S A E E K L W V T V Y Y G V P V W K E A T T T L F C A |
| 163 | TCCGATGCGAAGGCGCATCATGCGGAGGCGCATAATGTCTGGGCGACGCATGCGTGTGTCCCGACGGACCCGAACCCGCAA |
| 56 | S D A K A H H A E A M N V W A T K A C V P T D P N P Q |
| 244 | GAGGTCATTCTCGAGAATGTCACGGAGAAATATAACATGTGGAAAAATAACATGGTAGACCAGATGCATGAGGATATTATT |
| 82 | E V I L E N V T E K Y N M W K N N M V D Q M H E D I I |
| 325 | TCCCTCTGGGATCAATCCCTCAAGCCGTGTGTCAAACTCACGCCGCTCTGTGTCACGCTCAATTGCACGAATGCGACGTAT |
| 108 | S L W D Q S L K P C V K L T P L C V T L N C T N A T Y |
| 406 | ACGAATTCCGACTCCAAGAATTCCACTAGTAATTCCTCCCTCGAGGACTCCGGGAAAGGGGACATGAACTGCTCCTTCGAT |
| 136 | T N S D S K N S T S N S S L E D S G K G D M N C S F D |
| 487 | GTCACGACGTCCATTGATAAAAAGAAGAAGACGGAGTATGCGATTTTTGATAAACTCGATGTCATGAATATTGGGAATGGG |
| 163 | V T T S I D K K K K T E Y A I F D K L D V M N I G N G |
| 568 | CGGTATACGCTCCTCAATTGTAACACGTCCGTCATTACGCAGGCGTGTCCGAAGATGTCCTTTGAGCCGATTCCGATTCAT |
| 190 | R Y T L L N C N T S V I T Q A C P K M S F E P I P I H |
| 649 | TATTGTACGCCGGCGGGGTATGCGATTCTCAAGTGTAATGATAATAAGTTCAATGGGACGGGGCCGTGTACGAATGTCTCC |
| 217 | Y C T P A G Y A I L K C N D N K F N G T G P C T N V S |
| 730 | ACGATTCAATGTACGCATGGGATTAAGCCGGTCGTCTCCACGCAACTCCTCCTCAATGGATCCCTCGCGGAGGGGGGGGAG |
| 244 | T I Q C T H G I K P V V S T Q L L L N G S L A E G G E |
| 811 | GTCATTATTCGGTCCGAGAATCTCACGGACAATGCGAAAACGATTATTGTCCAGCTCAAGGAGCCGGTCGAGATTAATTGT |
| 271 | V I I R S E N L T D N A K T I I V Q L K E P V E I N C |
| 892 | ACGCGGCCGAACAACAATACGCGGAAATCCATTCATATGGGGCCGGGGGCGGCGTTTTATGCGCGGGGGGAGGTCATTGGG |
| 298 | T R P N N N T R K S I H M G P G A A F Y A R G E V I G |
| 973 | GATATTCGGCAAGCGCATTGCAACATTTCCCGGGGGCGGTGGAATGACACGCTCAAACAGATTGCGAAAAAACTCCGGGAG |
| 325 | D I R Q A H C N I S R G R W N D T L K Q I A K K L R E |
| 1054 | CAATTTAATAAAACGATTTCCCTCAACCAATCCTCCGGGGGGGACCTCGAGATTGTCATGCACACGTTTAATTGTGGGGGG |
| 352 | Q F N K T I S L N Q S S G G D L E I V M H T F N C G G |
| 1135 | GAGTTTTTCTACTGTAATACGACGCAGCTCTTTAATTCCACGTGGAATGAGAATGATACGACGTGGAATAATACGGCGGGG |
| 379 | E F F Y C N T T Q L F N S T W N E N D T T W N N T A G |
| 1216 | TCCAATAACAATGAGACGATTACGCTCCCGTGTCGGATTAAACAAATTATTAACCGGTGGCAGGAGGTCGGGAAAGCGATG |
| 406 | S N N N E T I T L P C R I K Q I I N R W Q E V G K A M |
| 1297 | TATGCGCCGCCGATTTCCGGGCCGATTAATTGTCTCTCCAATATTACGGGGCTCCTCCTCACGCGTGATGGGGGGGACAAC |
| 433 | Y A P P I S G P I N C L S N I T G L L L T R D G G D N |
| 1378 | AATAATACGATTGAGACGTTCCGGCCGGGGGGGGGGGATATGCGGGACAATTGGCGGTCCGAGCTCTATAAATATAAAGTC |
| 460 | N N T I E T F R P G G G D M R D N W R S E L Y K Y K V |
| 1459 | GTCCGGATTGAGCCGCTCGGGATTGCGCCGACGAACGCGAAGCGGCGGGTCGTCCAACGGGAGAAACGGGCGGTCGGGATT |
| 487 | V R I E P L G I A P T K A K R R V V Q R E K R A V G I |
| 1540 | GGGGCGATGTTCCTCGGGTTCCTCGGGGCGGCGGGGTCCACGATGGGGGCGGCGTCCGTCACGCTCACGGTCCAGGCGCGG |
| 514 | G A M F L G F L G A A G S T M G A A S V T L T V Q A R |
| 1621 | CTCCTCCTCTCCGGGATTGTCCAACAGCAAAACAATCTCCTCCGGGCGATTGAGGCGCAACAGCATCTCCTCCAACTCACG |
| 541 | L L L S G I V Q Q Q N N L L R A I E A Q Q H L L Q L T |
| 1702 | GTCTGGGGGATTAAGCAGCTCCAGGCGCGGGTCCTCGCGATGGAGCGGTACCTCAAGGATCAACAGCTCCTCGGGATTTGG |
| 568 | V W G I K Q L Q A R V L A M E R Y L K D Q Q L L G I W |
| 1788 | GGGTGCTCCGGGAAACTCATTTGCACGACGAATGTCCCGTGGAATGCGTCCTGGTCCAATAAATCCCTCGACAAGATTTGG |
| 595 | G C S G K L I C T T N V P W N A S W S N K S L D K I W |
| 1864 | CATAACATGACGTGGATGGAGTGGGACCGGGAGATTGACAATTACACGAAACTCATTTACACGCTCATTGAGGCGTCCCAG |
| 622 | H N M T W M E W D R E I D N Y T K L I Y T L I E A S Q |
| 1945 | ATTCAGCAGGAGAAGAATGAGCAAGAGCTCCTCGAGCTCGATTCCTGGGCGTCCCTCTGGTCCTGGTTTGACATTTCCAAA |
| 649 | I Q Q E K N E Q E L L E L D S W A S L W S W F D I S K |
| 2026 | TGGCTCTGGTATATTGGGGTCTTCATTATTGTCATTGGGGGGCTCGTCGGGCTCAAAATTGTCTTTGCGGTCCTCTCCATT |
| 676 | W L W Y I G V F I I V I G G L V G L K I V F A V L S I |
| 2107 | GTCAATCGGGTCCGGCAGGGGTACTCCCCGCTCTCCTTTCAGACGCGGCTCCCGGCGCCGCGGGGGCCGGACCGGCCGGAG |
| 703 | V N R V R Q G Y S P L S F Q T R L P A P R G P D R P E |
| 2188 | GGGATTGAGGAGGGGGGGGGGGAGCGGGACCGGGACAGATCTGATCAACTCGTCACGGGGTTCCTCGCGCTCATTTGGGAC |
| 730 | G I E E G G G E R D R D R S D Q L V T G F L A L I W D |
| 2269 | GATCTCCGGTCCCTCTGCCTCTTCTCCTACCACCGGCTCCGGGACCTCCTCCTCATTGTCGCGCGGATTGTCGAGCTCCTC |
| 757 | D L R S L C L F S Y H R L R D L L L I V A R I V E L L |
| 2350 | GGGCGGCGGGGGTGGGAGGCGCTCAAGTATTGGTGGAATCTCCTCCAATATTGGATTCAGGAGCTCAAGAATTCCGCGGTC |
| 784 | G R R G W E A L K Y W W N L L Q Y W I Q E L K N S A V |
| 2431 | TCCCTCCTCAACGCGACGGCGATTGCGGTCGCGGAGGGGACGGATCGGATTATTGAGGTCGTCCAACGGATTGGGCGGGCG |
| 811 | S L L N A T A I A V A E G T D R I I E V V Q R I G R A |
| 2512 | ATTCTCCACATTCCGCGGCGGATTCGGCAGGGGCTCGAGCGGGCGCTCCTCTAATGA |
| 833 | I L H I P R R I R Q G L E R A L L |

Gene assembly methods were identical to those employed above for HIV-1 gag. Since this gp160 gene exceeds 2.5 kb, it was assembled in 3 segments (each of approximately 800 bp-900 bp). A person skilled in the art would readily be able to select and assemble suitable segments.

The plasmid construct set forth in SEQ ID NO:9 was used as the vector for transfection of the modified polynucleotide prepared in accordance with an embodiment of the present invention.

| SEQ ID NO:9: | |
|---|---|
| aaatgggggc gctgaggtct gcctcgtgaa gaaggtgttg ctgactcata ccaggcctga | 60 |
| atcgccccat catccagcca gaaagtgagg gagccacggt tgatgagagc tttgttgtag | 120 |
| gtggaccagt tggtgatttt gaacttttgc tttgccacgg aacggtctgc gttgtcggga | 180 |
| agatgcgtga tctgatcctt caactcagca aaagttcgat ttattcaaca aagccgccgt | 240 |
| cccgtcaagt cagcgtaatg ctctgccagt gttacaacca attaaccaat tctgcgttca | 300 |
| aaatggtatg cgttttgaca catccactat atatccgtgt cgttctgtcc actcctgaat | 360 |
| cccattccag aaattctcta gcgattccag aagtttctca gagtcggaaa gttgaccaga | 420 |
| cattacgaac tggcacagat ggtcataacc tgaaggaaga tctgattgct taactgcttc | 480 |
| agttaagacc gacgcgctcg tcgtataaca gatgcgatga tgcagaccaa tcaacatggc | 540 |
| acctgccatt gctacctgta cagtcaagga tggtagaaat gttgtcggtc cttgcacacg | 600 |
| aatattacgc catttgcctg catattcaaa cagctcttct acgataaggg cacaaatcgc | 660 |
| atcgtggaac gtttgggctt ctaccgattt agcagtttga tacactttct ctaagtatcc | 720 |
| acctgaatca taaatcggca aaatagagaa aaattgacca tgtgtaagcg gccaatctga | 780 |
| ttccacctga gatgcataat ctagtagaat ctcttcgcta tcaaaattca cttccacctt | 840 |
| ccactcaccg gttgtccatt catggctgaa ctctgcttcc tctgttgaca tgacacacat | 900 |
| catctcaata tccgaatacg gaccatcagt ctgacgacca agagagccat aaacaccaat | 960 |
| agccttaaca tcatccccat atttatccaa tattcgttcc ttaatttcat gaacaatctt | 1020 |
| cattctttct tctctagtca ttattattgg tccgttcata acaccccttg tattactgtt | 1080 |
| tatgtaagca gacagtttta ttgttcatga tgatatattt ttatcttgtg caatgtaaca | 1140 |
| tcagagattt tgagacacaa cgtggctttc cccggcccat gaccaaaatc ccttaacgtg | 1200 |
| agttttcgtt ccactgagcg tcagaccccg tagaaaagat caaaggatct tcttgagatc | 1260 |
| ctttttttct gcgcgtaatc tgctgcttgc aaacaaaaaa accaccgcta ccagcggtgg | 1320 |
| tttgtttgcc ggatcaagag ctaccaactc tttttccgaa ggtaactggc ttcagcagag | 1380 |
| cgcagatacc aaatactgtc cttctagtgt agccgtagtt aggccaccac ttcaagaact | 1440 |
| ctgtagcacc gcctacatac ctcgctctgc taatcctgtt accagtggct gctgccagtg | 1500 |
| gcgataagtc gtgtcttacc gggttggact caagacgata gttaccggat aaggcgcagc | 1560 |
| ggtcgggctg aacggggggt tcgtgcacac agcccagctt ggagcgaacg acctacaccg | 1620 |
| aactgagata cctacagcgt gagctatgag aaagcgccac gcttcccgaa gggagaaagg | 1680 |
| cggacaggta tccggtaagc ggcagggtcg gaacaggaga gcgcacgagg gagcttccag | 1740 |
| ggggaaacgc ctggtatctt tatagtcctg tcgggtttcg ccacctctga cttgagcgtc | 1800 |
| gatttttgtg atgctcgtca ggggggcgga gcctatggaa aaacgccagc aacgcggcct | 1860 |
| ttttacggtt cctggccttt tgctggcctt ttgctcacat gttctttcct gcgttatccc | 1920 |
| ctgattctgt ggataaccgt attaccgcct ttgagtgagc tgataccgct cgccgcagcc | 1980 |
| gaacgaccga gcgcagcgag tcagtgagcg aggaagcgga agagcgcctg atgcggtatt | 2040 |
| ttctccttac gcatctgtgc ggtatttcac accgcatatg gtgcactctc agtacaatct | 2100 |
| gctctgatgc cgcatagtta agccagtatc tgctccctgc ttgtgtgttg gaggtcgctg | 2160 |
| agtagtgcgc gagcaaaatt taagctacaa caaggcaagg cttgaccgac aattgcatga | 2220 |
| agaatctgct tagggttagg cgttttgcgc tgcttcgcga tgtacgggcc agatatagcc | 2280 |
| gcggcatcga tgatatccat tgcatacgtt gtatctatat cataatatgt acatttatat | 2340 |
| tggctcatgt ccaatatgac cgccatgttg acattgatta ttgactagtt attaatagta | 2400 |
| atcaattacg gggtcattag ttcatagccc atatatggag ttccgcgtta cataacttac | 2460 |
| ggtaaatggc ccgcctggct gaccgcccaa cgacccccgc ccattgacgt caataatgac | 2520 |
| gtatgttccc atagtaacgc caatagggac tttccattga cgtcaatggg tggagtattt | 2580 |
| acggtaaact gcccacttgg cagtacatca agtgtatcat atgccaagtc cgccccctat | 2640 |
| tgacgtcaat gacggtaaat ggcccgcctg gcattatgcc cagtacatga ccttacggga | 2700 |
| ctttcctact tggcagtaca tctacgtatt agtcatcgct attaccatgg tgatgcggtt | 2760 |
| ttggcagtac atcaatgggc gtggatagcg gtttgactca cggggatttc caagtctcca | 2820 |
| ccccattgac gtcaatggga gtttgttttg gcaccaaaat caacgggact ttccaaaatg | 2880 |
| tcgtaacaac tccgccccat tgacgcaaat gggcggtagg cgtgtacggt gggaggtcta | 2940 |
| tataagcaga gctcgtttag tgaaccgtca gatcgcctgg agacgccatc cacgctgttt | 3000 |
| tgacctccat agaagacacc gggaccgatc cagcctccgc gggcgcgcgt cgacgccacc | 3060 |
| atgcgggcga aggagatgcg gaagtcctgt cagcacctcc ggaaatgggg gattctcctc | 3120 |
| tttggggtcc tcatgatttg ttccgcggag gagaagctct gggtcacggt ctattatggg | 3180 |
| gtcccggtct ggaaagaggc gacgacgacg ctcttttgtg cgtccgatgc gaaggcgcat | 3240 |
| catgcggagg cgcataatgt ctgggcgacg catgcgtgtg tcccgacgga cccgaacccg | 3300 |
| caagaggtca ttctcgagaa tgtcacggag aaatataaca tgtggaaaaa taacatggta | 3360 |
| gaccagatgc atgaggatat tatttccctc tgggatcaat ccctcaagcc gtgtgtcaaa | 3420 |
| ctcacgccgc tctgtgtcac gctcaattgc acgaatgcga cgtatacgaa ttccgactcc | 3480 |
| aagaattcca ctagtaattc ctccctcgag gactccggga aaggggacat gaactgctcc | 3540 |
| ttcgatgtca cgacgtccat tgataaaaag aagaagacgg agtatgcgat ttttgataaa | 3600 |
| ctcgatgtca tgaatattgg gaatgggcgg tatacgctcc tcaattgtaa cacgtccgtc | 3660 |
| attacgcagg cgtgtccgaa gatgtccttt gagccgattc cgattcatta ttgtacgccg | 3720 |
| gcggggtatg cgattctcaa gtgtaatgat aataagttca atgggacggg gccgtgtacg | 3780 |
| aatgtctcca cgattcaatg tacgcatggg attaagccgg tcgtctccac gcaactcctc | 3840 |
| ctcaatggat ccctcgcgga ggggggggag gtcattattc ggtccgagaa tctcacggac | 3900 |
| aatgcgaaaa cgattattgt ccagctcaag gagccggtcg agattaattg tacgcggccg | 3960 |
| aacaacaata cgcggaaatc cattcatatg gggccggggg cggcgtttta tgcgcggggg | 4020 |
| gaggtcattg gggatattcg gcaagcgcat tgcaacattt cccgggggcg gtggaatgac | 4080 |
| acgctcaaac agattgcgaa aaaactccgg gagcaattta ataaaacgat ttccctcaac | 4140 |
| caatcctccg ggggggacct cgagattgtc atgcacacgt ttaattgtgg gggggagttt | 4200 |
| ttctactgta atacgacgca gctctttaat tccacgtgga atgagaatga tacgacgtgg | 4260 |
| aataatacgg cggggtccaa taacaatgag acgattacgc tcccgtgtcg gattaaacaa | 4320 |
| attattaacc ggtggcagga ggtcgggaaa gcgatgtatg cgccgccgat ttccgggccg | 4380 |
| attaattgtc tctccaatat tacggggctc ctcctcacgc gtgatggggg ggacaacaat | 4440 |
| aatacgattg agacgttccg gccggggggg ggggatatgc gggacaattg gcggtccgag | 4500 |
| ctctataaat ataaagtcgt ccggattgag ccgctcggga ttgcgccgac gaaggcgaag | 4560 |
| cggcgggtcg tccaacggga gaaacgggcg gtcgggattg gggcgatgtt cctcgggttc | 4620 |
| ctcggggcgg cggggtccac gatgggggcg gcgtccgtca cgctcacggt ccaggcgcgg | 4680 |
| ctcctcctct ccgggattgt ccaacagcaa aacaatctcc tccgggcgat tgaggcgcaa | 4740 |
| cagcatctcc tccaactcac ggtctggggg attaagcagc tccaggcgcg ggtcctcgcg | 4800 |
| atggagcggt acctcaagga tcaacagctc ctcgggattt gggggtgctc cgggaaactc | 4860 |
| atttgcacga cgaatgtccc gtggaatgcg tcctggtcca ataaatccct cgacaagatt | 4920 |
| tggcataaca tgacgtggat ggagtgggac cgggagattg acaattacac gaaactcatt | 4980 |
| tacacgctca ttgaggcgtc ccagattcag caggagaaga atgagcaaga gctcctcgag | 5040 |
| ctcgattcct gggcgtccct ctggtcctgg tttgacattt ccaaatggct ctggtatatt | 5100 |
| ggggtcttca ttattgtcat tggggggctc gtcgggctca aaattgtctt tgcggtcctc | 5160 |
| tccattgtca atcgggtccg gcaggggtac tccccgctct cctttcagac gcggctcccg | 5220 |
| gcgccgcggg ggccggaccg gccggagggg attgaggagg ggggggggga gcgggaccgg | 5280 |
| gacagatctg atcaactcgt cacggggttc ctcgcgctca tttgggacga tctccggtcc | 5340 |
| ctctgcctct tctcctacca ccggctccgg gacctcctcc tcattgtcgc gcggattgtc | 5400 |
| gagctcctcg ggcggcgggg gtgggaggcg ctcaagtatt ggtggaatct cctccaatat | 5460 |
| tggattcagg agctcaagaa ttccgcggtc tccctcctca acgcgacggc gattgcggtc | 5520 |
| gcggagggga cggatcggat tattgaggtc gtccaacgga ttgggcgggc gattctccac | 5580 |
| attccgcggc ggattcggca ggggctcgag cgggcgctcc tctaatgagg cgcgccgagc | 5640 |
| tcgctgatca gcctcgactg tgccttctag ttgccagcca tctgttgttt gcccctcccc | 5700 |
| cgtgccttcc ttgaccctgg aaggtgccac tcccactgtc ctttcctaat aaaatgagga | 5760 |
| aattgcatcg cattgtctga gtaggtgtca ttctattctg gggggtgggg tggggcagga | 5820 |
| cagcaagggg gaggattggg aagacaatag caggcatgct ggggaattt | 5869 |

The plasmid construct set forth in SEQ ID NO:10 is the vector for the transfection of the wild-type gene.

| SEQ ID NO:10: | |
|---|---|
| aaatgggggc gctgaggtct gcctcgtgaa gaaggtgttg ctgactcata ccaggcctga | 60 |
| atcgccccat catccagcca gaaagtgagg gagccacggt tgatgagagc tttgttgtag | 120 |
| gtggaccagt tggtgatttt gaacttttgc tttgccacgg aacggtctgc gttgtcggga | 180 |
| agatgcgtga tctgatcctt caactcagca aaagttcgat ttattcaaca aagccgccgt | 240 |
| cccgtcaagt cagcgtaatg ctctgccagt gttacaacca attaaccaat tctgcgttca | 300 |
| aaatggtatg cgttttgaca catccactat atatccgtgt cgttctgtcc actcctgaat | 360 |
| cccattccag aaattctcta gcgattccag aagtttctca gagtcggaaa gttgaccaga | 420 |
| cattacgaac tggcacagat ggtcataacc tgaaggaaga tctgattgct taactgcttc | 480 |
| agttaagacc gacgcgctcg tcgtataaca gatgcgatga tgcagaccaa tcaacatggc | 540 |
| acctgccatt gctacctgta cagtcaagga tggtagaaat gttgtcggtc cttgcacacg | 600 |
| aatattacgc catttgcctg catattcaaa cagctcttct acgataaggg cacaaatcgc | 660 |
| atcgtggaac gtttgggctt ctaccgattt agcagtttga tacactttct ctaagtatcc | 720 |
| acctgaatca taaatcggca aaatagagaa aaattgacca tgtgtaagcg gccaatctga | 780 |
| ttccacctga gatgcataat ctagtagaat ctcttcgcta tcaaaattca cttccacctt | 840 |
| ccactcaccg gttgtccatt catggctgaa ctctgcttcc tctgttgaca tgacacacat | 900 |
| catctcaata tccgaatacg gaccatcagt ctgacgacca agagagccat aaacaccaat | 960 |
| agccttaaca tcatccccat atttatccaa tattcgttcc ttaatttcat gaacaatctt | 1020 |
| cattctttct tctctagtca ttattattgg tccgttcata acaccccttg tattactgtt | 1080 |
| tatgtaagca gacagtttta ttgttcatga tgatatattt ttatcttgtg caatgtaaca | 1140 |
| tcagagattt tgagacacaa cgtggctttc cccggcccat gaccaaaatc ccttaacgtg | 1200 |
| agttttcgtt ccactgagcg tcagaccccg tagaaaagat caaaggatct tcttgagatc | 1260 |
| ctttttttct gcgcgtaatc tgctgcttgc aaacaaaaaa accaccgcta ccagcggtgg | 1320 |
| tttgtttgcc ggatcaagag ctaccaactc tttttccgaa ggtaactggc ttcagcagag | 1380 |
| cgcagatacc aaatactgtc cttctagtgt agccgtagtt aggccaccac ttcaagaact | 1440 |
| ctgtagcacc gcctacatac ctcgctctgc taatcctgtt accagtggct gctgccagtg | 1500 |
| gcgataagtc gtgtcttacc gggttggact caagacgata gttaccggat aaggcgcagc | 1560 |
| ggtcgggctg aacggggggt tcgtgcacac agcccagctt ggagcgaacg acctacaccg | 1620 |
| aactgagata cctacagcgt gagctatgag aaagcgccac gcttcccgaa gggagaaagg | 1680 |
| cggacaggta tccggtaagc ggcagggtcg gaacaggaga gcgcacgagg gagcttccag | 1740 |
| ggggaaacgc ctggtatctt tatagtcctg tcgggtttcg ccacctctga cttgagcgtc | 1800 |
| gatttttgtg atgctcgtca ggggggcgga gcctatggaa aaacgccagc aacgcggcct | 1860 |
| ttttacggtt cctggccttt tgctggcctt ttgctcacat gttctttcct gcgttatccc | 1920 |
| ctgattctgt ggataaccgt attaccgcct ttgagtgagc tgataccgct cgccgcagcc | 1980 |
| gaacgaccga gcgcagcgag tcagtgagcg aggaagcgga agagcgcctg atgcggtatt | 2040 |
| ttctccttac gcatctgtgc ggtatttcac accgcatatg gtgcactctc agtacaatct | 2100 |
| gctctgatgc cgcatagtta agccagtatc tgctccctgc ttgtgtgttg gaggtcgctg | 2160 |
| agtagtgcgc gagcaaaatt taagctacaa caaggcaagg cttgaccgac aattgcatga | 2220 |
| agaatctgct tagggttagg cgttttgcgc tgcttcgcga tgtacgggcc agatatagcc | 2280 |
| gcggcatcga tgatatcgcg gctatctgag gggactaggg tgtgtttagg cgaaaagcgg | 2340 |
| ggcttcggtt gtacgcggtt aggagtcccc tcaccattgc atacgttgta tctatatcat | 2400 |
| aatatgtaca tttatattgg ctcatgtcca atatgaccgc catgttgaca ttgattattg | 2460 |
| actagttatt aatagtaatc aattacgggg tcattagttc atagcccata tatggagttc | 2520 |
| cgcgttacat aacttacggt aaatggcccg cctggctgac cgcccaacga cccccgccca | 2580 |
| ttgacgtcaa taatgacgta tgttcccata gtaacgccaa tagggacttt ccattgacgt | 2640 |
| caatgggtgg agtatttacg gtaaactgcc cacttggcag tacatcaagt gtatcatatg | 2700 |
| ccaagtccgc cccctattga cgtcaatgac ggtaaatggc ccgcctggca ttatgcccag | 2760 |
| tacatgacct tacgggactt tcctacttgg cagtacatct acgtattagt catcgctatt | 2820 |
| accatggtga tgcggttttg gcagtacatc aatgggcgtg gatagcggtt tgactcacgg | 2880 |
| ggatttccaa gtctccaccc cattgacgtc aatgggagtt tgttttggca ccaaaatcaa | 2940 |
| cgggactttc caaaatgtcg taacaactcc gccccattga cgcaaatggg cggtaggcgt | 3000 |
| gtacggtggg aggtctatat aagcagagct cgtttagtga accgtcagat cgcctggaga | 3060 |
| cgccatccac gctgttttga cctccataga agacaccggg accgatccag cctccgcggg | 3120 |
| cgcgcgtcga cgccaccatg agagcgaagg agatgaggaa gagttgtcag cacttgagga | 3180 |
| aatggggcat cttgctcttt ggagtgttga tgatctgtag tgctgaagaa aagttgtggg | 3240 |
| tcacagtcta ttatggggta cctgtgtgga aagaagcaac caccactcta ttttgtgcat | 3300 |
| cagatgctaa ggcacatcat gcagaggcac ataatgtttg ggccacacat gcctgtgtac | 3360 |
| ccacagaccc taacccacaa gaagtaatat tggaaaatgt gacagaaaaa tataacatgt | 3420 |
| ggaaaaataa catggtagac cagatgcatg aggatataat cagtttatgg gatcaaagcc | 3480 |
| taaagccatg tgtaaaatta accccactct gtgttacttt aaattgcact aatgcgacgt | 3540 |
| atactaatag tgacagtaag aatagtacca gtaatagtag tttggaagac agtgggaaag | 3600 |
| gagacatgaa ctgctctttc gatgtcacca caagcataga taaaaagaag aagacagaat | 3660 |
| atgcaatttt tgataaactt gatgtaatga atataggtaa tggaagatat acattactaa | 3720 |
| attgtaacac ctcagtcatt acacaggcct gtccaaagat gtcctttgaa ccaattccca | 3780 |
| tacattattg taccccggct ggttatgcga ttctaaagtg taatgataat aagttcaatg | 3840 |
| gaacaggacc atgtacaaat gtcagcacaa tacaatgtac acatggaatt aagccagtag | 3900 |
| tgtcaactca actgctgtta aatggcagtc tagcagaagg aggagaggta ataattagat | 3960 |
| ctgaaaatct cacagacaat gctaaaacca taatagtaca gctcaaggaa cctgtagaaa | 4020 |
| tcaattgtac aagacccaac aacaatacaa gaaaaagtat acatatggga ccaggagcag | 4080 |
| cattttatgc aagaggagaa gtaataggag atataagaca agcacattgc aacattagta | 4140 |
| gaggaagatg gaatgacact ttaaaacaga tagctaaaaa attaagagaa caatttaata | 4200 |
| aaacaataag ccttaaccaa tcctcaggag gggacctaga aattgtaatg cacactttta | 4260 |
| attgtggagg ggaatttttc tactgtaata caacacagct gtttaatagt acttggaatg | 4320 |
| agaatgatac tacctggaat aatacagcag ggtcaaataa caatgaaact atcacactcc | 4380 |
| catgtagaat aaaacaaatt ataaacaggt ggcaggaagt aggaaaagca atgtatgccc | 4440 |
| ctcccatcag tggaccaatt aattgtttat caaatatcac agggctatta ttaacaagag | 4500 |
| atggtggtga caacaataat acaatagaga ccttcagacc tggaggagga gatatgaggg | 4560 |
| acaattggag aagtgaatta tataaatata aagtagtaag aattgagcca ttaggaatag | 4620 |
| cacccaccaa ggcaaagaga agagtggtgc aaagagaaaa aagagcagtg ggaataggag | 4680 |
| ctatgttcct tgggttcttg ggagcagcag gaagcactat gggcgcagcg tcagtgacgc | 4740 |
| tgacggtaca ggccagacta ttattgtctg gtatagtgca acagcaaaac aatttgctga | 4800 |
| gagctatcga ggcgcaacag catctgttgc aactcacagt ctggggcatc aagcagctcc | 4860 |
| aggctagagt cctggctatg gaaagatacc taaaggatca acagctccta gggatttggg | 4920 |
| gttgctctgg aaaactcatt tgcaccacta atgtgccttg gaatgctagt tggagtaata | 4980 |
| aatctctgga caagatttgg cataacatga cctggatgga gtgggacaga gaaattgaca | 5040 |
| attacacaaa attaatatac accttaattg aagcatcgca gatccagcag gaaaagaatg | 5100 |
| aacaagaatt attggaattg gatagttggg caagtttgtg gagttggttt gacatctcaa | 5160 |
| aatggctgtg gtatatagga gtattcataa tagtaatagg aggtttagta ggtttaaaaa | 5220 |
| tagtttttgc tgtactttct atagtaaata gagttaggca gggatactca ccattatcat | 5280 |
| ttcagacccg cctcccagcc ccgcggggac ccgacaggcc cgaaggaatc gaagaaggag | 5340 |
| gtggagagag agacagagac agatccgatc aattagtgac tggattctta gcactcatct | 5400 |
| gggacgatct gcggagcctg tgcctcttca gctaccaccg cttgagagac ttactcttga | 5460 |
| ttgtagcgag gattgtggaa cttctgggac gcagggggtg ggaagccctg aagtattggt | 5520 |
| ggaatctcct gcaatattgg attcaggaac taaagaatag tgctgttagt ttgcttaacg | 5580 |
| ccacagctat agcagtagcc gaggggacag ataggattat agaagtagta caaaggattg | 5640 |
| gtagagctat tctccacata cctagaagaa taagacaggg cttagaaagg gctttgctat | 5700 |
| aatagggcgc gccgagctcg ctgatcagcc tcgactgtgc cttctagttg ccagccatct | 5760 |
| gttgtttgcc cctcccccgt gccttccttg accctggaag gtgccactcc cactgtcctt | 5820 |
| tcctaataaa atgaggaaat tgcatcgcat tgtctgagta ggtgtcattc tattctgggg | 5880 |
| ggtggggtgg ggcaggacag caagggggag gattgggaag acaatagcag gcatgctggg | 5940 |
| gaattt | 5946 |

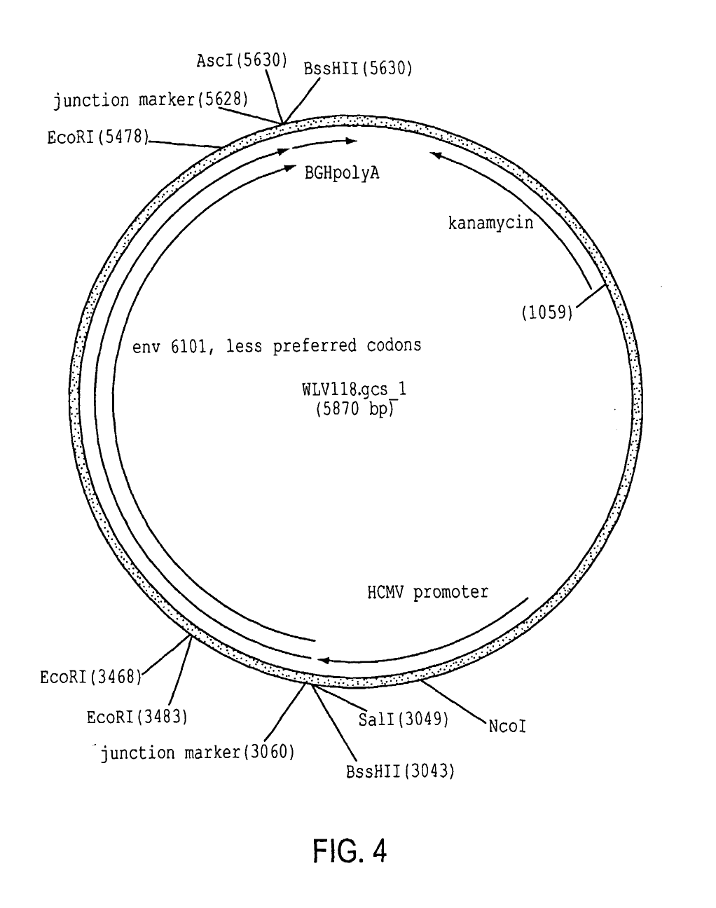

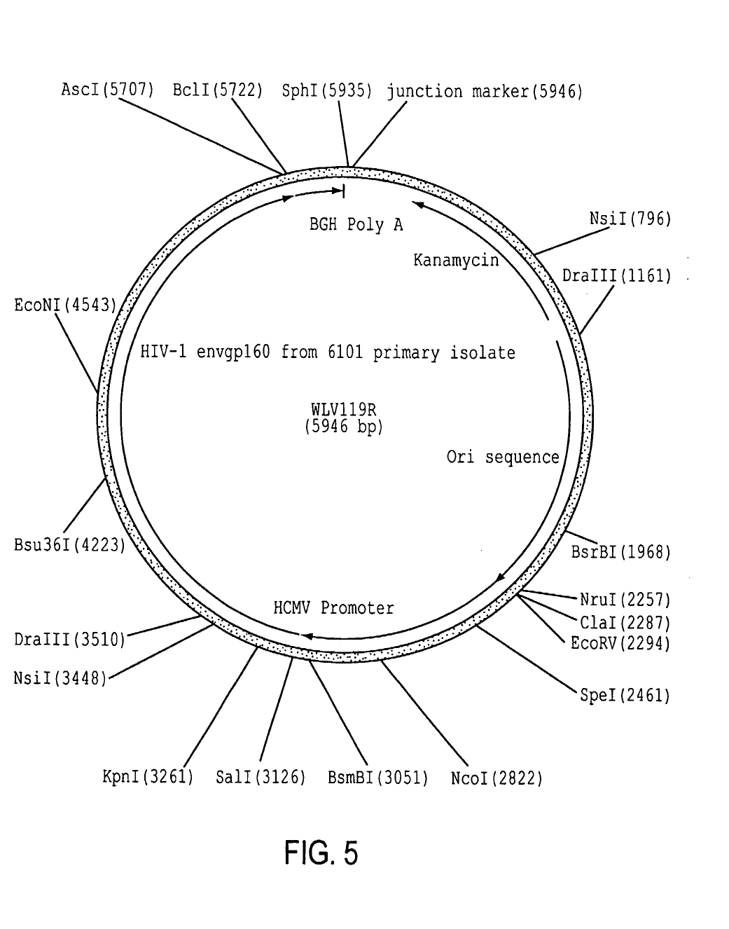

A plasmid map of the plasmid construct set forth in SEQ ID NO:9 is provided as FIG. 4 and a plasmid map of the plasmid construct set forth in SEQ ID NO:10 is provided as FIG. 5.

Western blot detection and ELISA methods were employed to compare transfected cells expressing the wild type or the modified gp160 genes.

Two Western blots confirmed gp160 antigen specificity from SEQ ID NO:9 plasmid construct-transfected 293 cells forty eight hours later (data not shown). Initial studies tested two SEQ ID NO:9 plasmid construct clones with later focus on clone 6, hereafter just denoted SEQ ID NO:9. These Western blots demonstrated recognition of SEQ ID NO: 9 plasmid construct-transfected lysates by both an anti IIIB gp120 polyclonal rabbit serum as well as an anti-MN gp41 monoclonal antibody (data not shown). Each blot revealed reactivity with their respective positive control recombinant proteins (451 for gp160 and MN expressed in E. coli for gp41. Since the amino acid sequences differ between the 6101 primary isolate (encoded by the SEQ ID NO:9 plasmid construct) and the MN strain, no direct quantitative comparisons can be made between these envelopes in these Western blots or in the ELISA assays listed below.

Enhanced expression levels of the 6101 gp160 envelope gene according to an embodiment of the present invention was observed. The plasmid construct for the gene modified in accordance with an embodiment of the present invention (SEQ ID NO:9) expressed substantially higher levels of gp160 compared to the wild-type 6101 gene (which was undetectable by Western blot). Envelope 6101 gp160 expression levels were quantified for 293 as well as for COS-7, Hela, and RD cell lines after transient transfection from total cell lysates using an anti-gp120 ELISA capture kit (ABI, Cat No. 15-102-000).

TABLE IV
HIV-1 Gp160 6101 protein levels (in ng/ml) from total cell lysates

| | Cells | | | |
|---|---|---|---|---|
| Constructs | COS-7 | Hela | RD | 293 |
| construct for modified polynucleotide | 4 | 5.4 | 0.8 | 80 |
| (SEQ ID NO: 9) | | | | |
| construct for wild-type | ** | ** | ** | ** |
| (SEQ ID NO: 10) | | | | |

*Lower limit of standard curve = 78 pg/ml
**not detected

From these studies it can be concluded that the construct for the modified gene (SEQ ID NO: 9) expresses the altered 6101 gp160 protein at levels far superior (almost 100 times) to its wild-type counterpart (SEQ ID NO:10) in several cell lines (as shown in Table IV). Quantification of this primary isolate can be achieved by an ABI anti-gp120 ELISA kit and is at substantially lower levels than observed for p37 gag (in the ug/ml range in cell lysates).

### Example 4

### Modification of the Env Gene Increased gp160 Protein Levels Relative to Wild-Type

A further study comparing the expression of a modified polynucleotide of an embodiment of the present invention for gp160 to the wild-type version of the gene was conducted.

For the purposes of the study, a modified polynucleotide of an embodiment of the present invention for gp160 was prepared as described in Example 3 above. A wild-type gp160 polynucleotide for the gene was also obtained for the study.

Expression of the two types of polynucleotides was measured using the systems described in Examples 1-3 above.

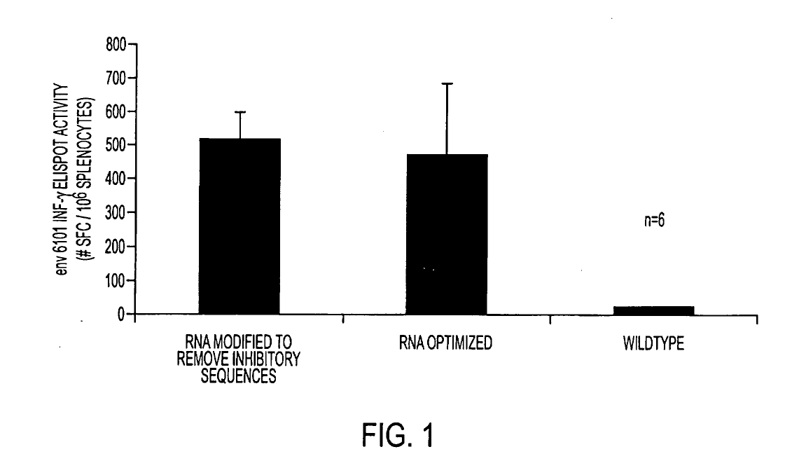

Referring to FIG. 1, the results of the study are illustrated by the graph. As is clearly shown, the modified polynucleotide of an embodiment of the present invention for the gp160 (“optimized”) gene provides substantially better expression than the wild-type gene.

### Example 5

### Enhanced Expression of Human IL-15

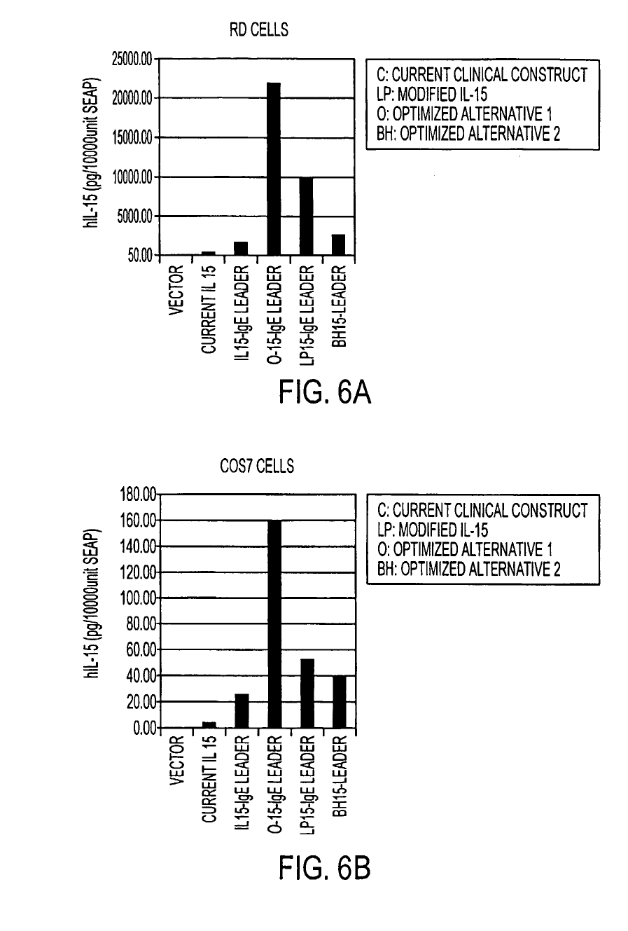

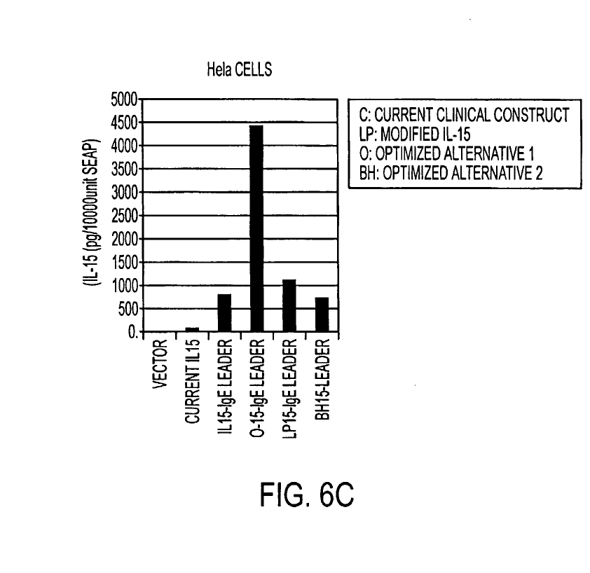

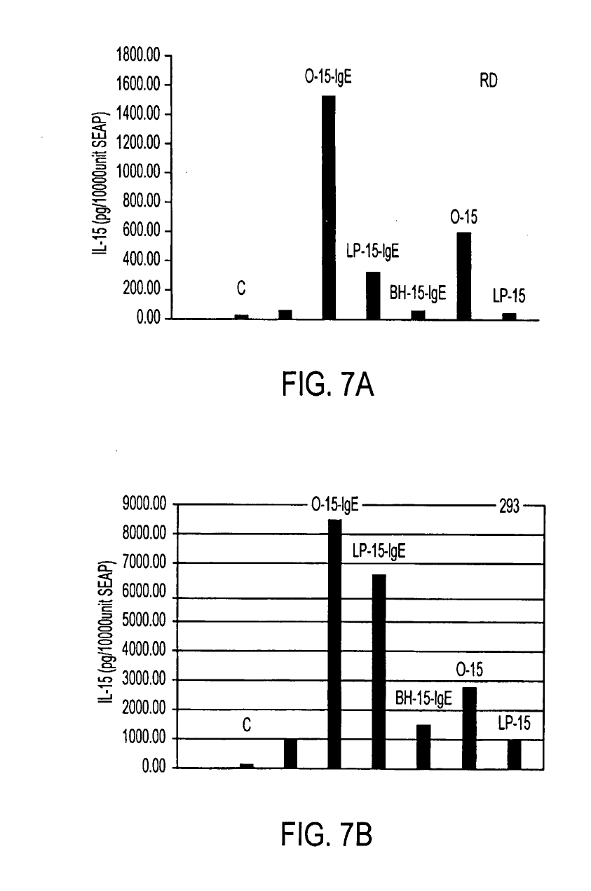

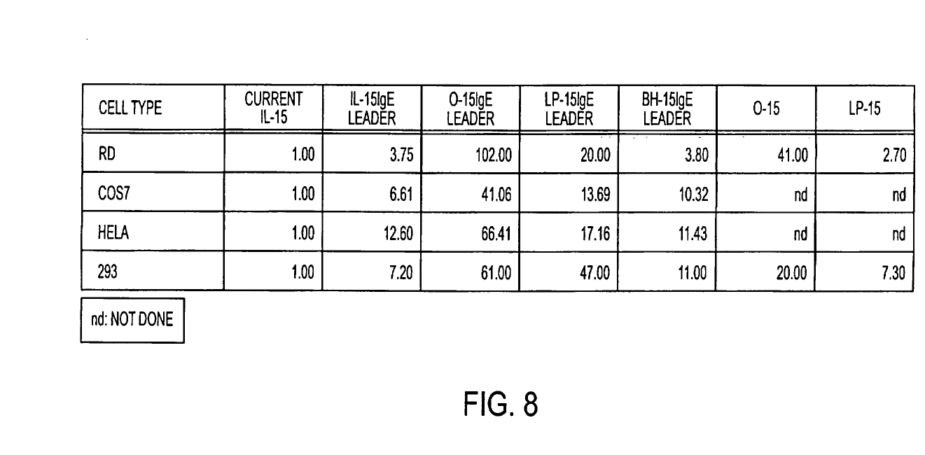

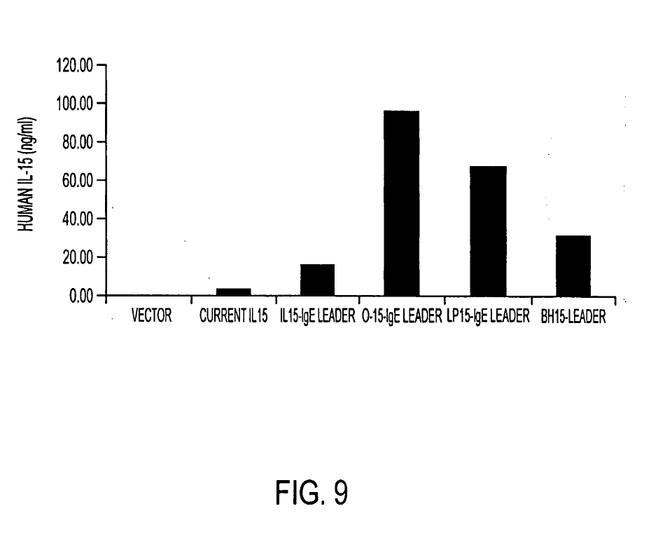

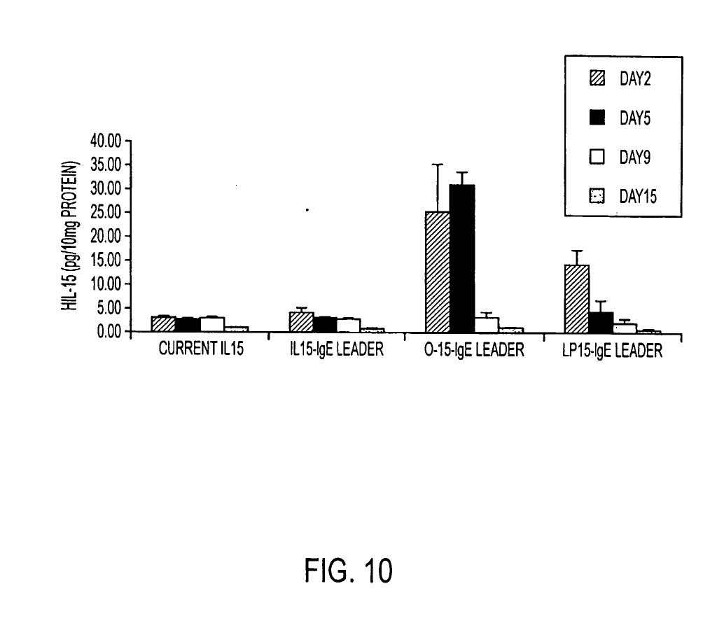

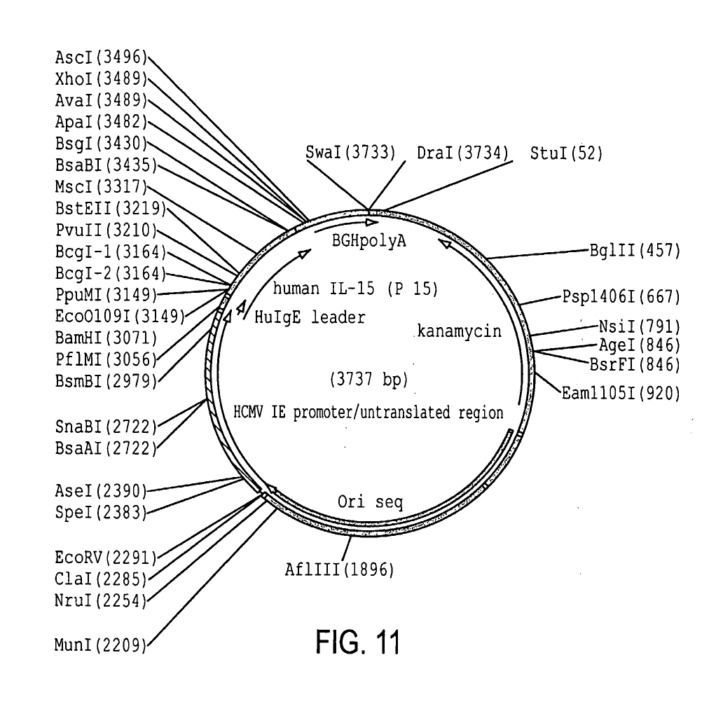

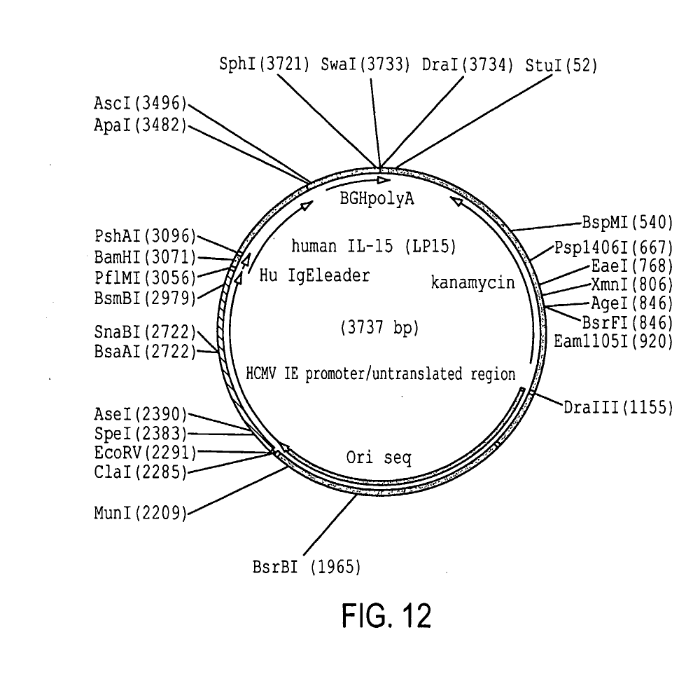

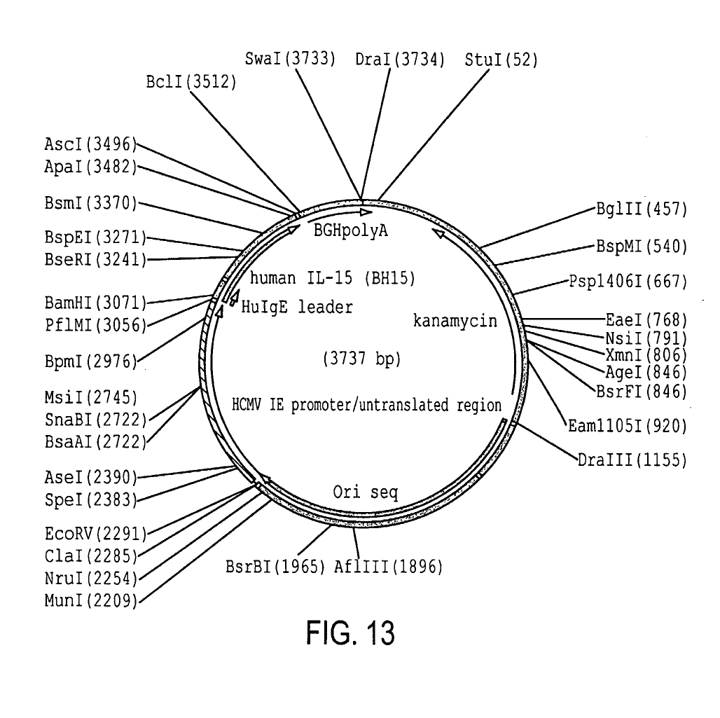

A study was conducted to compare IL-15 expression by various IL-15 constructs in accordance with embodiments of the present invention, such as an IL-15 recombinant construct (modified with surrogate codons) with a human IgE leader sequence or with the long leader sequence, unmodified IL-15 with an IgE leader, and two alternative optimized IL-15 constructs with IgE leader against expression by other IL-15 constructs. The results of the study show that the constructs of the present invention provide unexpectedly improved expression of IL-15. In particular, the IgE leader sequence in combination with the less intensive modified surrogate codon approach provides synergistically improved expression over currently used IL-15 constructs and comparable results to codon optimized or “preferred codon” approaches with a lower intensive and thus highly efficient and accurate approach. The experimental procedures and results are described below and illustrated in the following Tables and in FIGS. 6-10.

Various constructs were used for comparative purposes, as follows:

1. IL-15 constructs with the native IL-15 signal peptide replaced by the human IgE leader sequence.

2. IL-15 constructs with optimized codons (codon optimization alternative 1.

3. IL-15 constructs with the IL-15 nucleotide sequence optimized to reduce mRNA secondary structure (codon optimization alternative 2).

4. IL-15 constructs with combinations of IgE leader sequences and gene optimization techniques.

### Cloning:

All gene sequences were designed based upon published codon tables and synthesized from Blue Heron Technologies. Genes were then subcloned into the DNA vaccine vector backbone.

### Cell Culture and Transfection:

RD, 293, Hela and COS-7 cells were used in transient transfections. All transfections were carried out using Fugene-6 (Roche) according to the manufacturer's instructions. A total of 0.25 mg of human IL-15 plasmid and 0.5 mg of SEAP (a secreted form of human placental alkaline phosphatase) control vector with 4 ml of Fugene-6 was used for each transfection. For dose titration, 0.25-2.0 mg of the test plasmid was used along with the control DNA and the total DNA was made up to a final concentration of 2.0 mg per transfection. Dose titration was performed to identify an appropriate concentration of plasmid to be used for comparative analysis. Forty-eight hours after transfection, cell culture media and cells were harvested and analyzed for IL-15 by ELISA (R&D Systems) and CTLL2 proliferation assay. The cell lysates were tested for total protein concentration by Micro BCA protein assay. Data is depicted as pg of IL-15 per mg of protein in cell lysates and pg of IL-15 per 10,000 units of seap activity.

### Intramuscular Immunization of Mice:

Six to eight-week-old female BALB/c mice were used in this study. Each group consisted of 2 animals and mice were immunized intramuscularly in both quadriceps muscles with a total of 200 mg plasmid DNA (formulated with 0.25% bupivacaine) in a 50 ml volume using a 28-gauge needle. In all 4 muscles were analysed at each time point. The quadriceps muscles were taken at 2, 5, 9 and 15 days post-immunization and homogenized in cell lysis buffer (50 mM Tris, pH8.0-50 mM NaCl-1% Triton-X100) containing proteinase inhibitor mixture (Roche). The cell lysates were subjected to three freeze and thaw cycles, centrifuged and supernatants were evaluated for IL-15 protein by ELISA (R&D Systems). Data are represented as average expression in 4 muscle samples per group.

### CTLL2 Cell Proliferation Assay

Mouse CTLL2 cells were washed twice with PBS and incubated in a 96 well-plate at a density of 100000 cells/well in complete medium with either different amounts of human recombinant IL-15 (R&D Systems) as standard controls or indicated media of cells transfected with hIL-15 expression construct. Forty eight hours post-incubation, MTT reagent (3-(4,5-dimethylthiazolyl-2)-2,5-diphenyltetrazolium bromide) was added and further incubated for four hours. Conversion of the tetrazolium salt to the purple formazon product by mitochondrial enzymes in viable cells allows a visual assessment of the reaction. When the purple formazon precipitate was clearly visible in the microscope the cells were lysed with the detergent and absorbances read at 570 nm. Final concentration is based upon the known standards used in the assay and data are represented as pg of IL-15 per ml of supernatant from transfected cells.

### Results:

### Human IL-15 Constructs:

The following seven human IL-15 inserts were subcloned into a vector backbone, which contains human CMV promoter. All the constructs were confirmed by sequencing and used for in vitro and in vivo human IL-15 expression assays.

| | |
|---|---|
| +++++ | LP-IL-15-IgE leader (surrogate codons) |
| −−−−−−−−− | Current clinical IL-15 (native IL-15 with long |
| | signal peptide) |
| +++++ | Native IL-15 with IL-15-IgE leader that replaces |
| | the long signal peptide |
| +++++ | O-IL-15-IgE leader (preffered codons) |
| +++++ | BH-IL-15-IgE leader (secondary structure |
| | optimization) |
| −−−−−−−−− | O-15 with a long signal peptide |
| −−−−−−−−− | LP-15 with a long signal peptide |
| −−−−−−−−− | RNA optimization with a long signal peptide |

−−−−−− Native Leader Sequence
+++++ IgE Leader Sequence

As shown in Table V(A) and V(B), constructs according to embodiments of the present invention significantly improve IL-15 expression in vitro. In particular, Table V(A) shows expression in cells and supernatants of 293 cells. Table V(B) shows expression in cells and supernatants of RD cells

(A)
Human IL-15 expression in 293 cell lysates (ELISA)

| | | Fold increase |
|---|---|---|
| | human IL15 (pg/mg | compared to |
| Group | protein) | WLV125M |
| WLV125M | 7139.83 | 1.00 |
| WLV134M | 23893.23 | 3.35 |
| WLV186M | 123002.31 | 17.23 |
| WLV187M | 80523.75 | 11.28 |
| WLV188M | 29772.71 | 4.17 |
| WLV211M | 33000.66 | 4.62 |
| WLV217M | 11403.65 | 1.60 |
| WLV225M | 29103.13 | 4.08 |
| WLV001AM | 0.00 | 0 |

Human IL-15 expression in 293 cell supernatants (ELISA)

| | human IL15 | Fold increase |
|---|---|---|
| | (pg/ml/10000 unit | compared to |
| Group | SEAP) | WLV125M |
| WLV125M | 64.24 | 1.00 |
| WLV134M | 928.76 | 14.46 |
| WLV186M | 6807.04 | 105.96 |
| WLV187M | 4389.32 | 68.33 |
| WLV188M | 1327.20 | 20.66 |
| WLV211M | 967.94 | 15.07 |
| WLV217M | 217.81 | 3.39 |
| WLV225M | 1556.50 | 24.23 |
| WLV001AM | 0.00 | 0 |

(B)

| Human IL-15 expression in | | | Human IL-15 expression | | |
|---|---|---|---|---|---|
| RD cell supernatants (ELISA) | | | in RD cell lysates (ELISA) | | |
| | human IL15 | Fold increase | | | Fold increase |
| | (pg/ml/10000 unit | compared to | | human IL15 (pg/mg | compared to |
| Group | SEAP) | WLV125M | Group | protein) | WLV125M |
| WLV125M | 72.97 | 1.00 | WLV125M | 1056.64 | 1 |
| WLV134M | 528.40 | 7.24 | WLV134M | 2786.32 | 2 |
| WLV186M | 9544.01 | 130.79 | WLV186M | 20877.53 | 19 |
| WLV187M | 4102.73 | 56.22 | WLV187M | 7287.57 | 6 |
| WLV188M | 1548.02 | 21.21 | WLV188M | 3275.43 | 3 |
| WLV211M | 6287.93 | 86.17 | WLV211M | 6183.53 | 5 |
| WLV217M | 407.16 | 5.58 | WLV217M | 1409.34 | 1 |
| WLV225M | 1958.41 | 26.84 | WLV225M | 4443.84 | 4 |
| WLV001AM | 0.00 | 0 | WLV001AM | 0.00 | |

Table VI shows in vivo gene expression from IL-15 constructs in accordance with the invention as well as previously used IL-15 constructs for purposes of comparison. Codon engineering in addition to the replacement of the native signal peptide with human IgE leader significantly improved IL-15 expression in vivo. Four mice per group received 200 mg of plasmid DNA. Animals were sacrificed and analyzed at 2, 5, 9 and 15 days after immunization. Data summarized are an average IL-15 protein expression from a group of 4 muscles per time point.

Human IL-15 expression in the mouse
muscles(pg/10 mg of protein)

| Groups | Day 2 | Day 5 | Day 9 | Day 15 |
|---|---|---|---|---|
| WLV125M | 2.959 | 2.714 | 2.889 | 0.845 |
| WLV134M | 4.134 | 3.028 | 2.927 | 0.811 |
| WLV186M | 25.846 | 31.830 | 3.403 | 1.220 |
| WLV187M | 15.072 | 4.826 | 2.499 | 0.829 |

Table VII shows the results of the CTLL2 assay. Supernatants from RD cells transfected with optimized constructs induced 5-30 fold higher functional IL-15 than the native plasmid in a MTT cell proliferation bioassay (see materials and methods for details). The proliferation rate was estimated from a standard curve obtained with purified recombinant human IL-15 (pg/ml).

Human IL-15 expression in 293 cell lysates (CTLL2 Assay)

| | | Fold increase |
|---|---|---|
| | human IL15 (ng/ml of | compared to |
| Group | supernatant) | WLV125M |
| WLV125M | 3.12 | 1.00 |
| WLV134M | 16.22 | 5.19 |
| WLV186M | 98.95 | 31.69 |
| WLV187M | 71.42 | 22.87 |
| WLV188M | 34.36 | 11.01 |
| WLV001AM | 0.00 | 0.00 |

The foregoing study demonstrates that various gene modification strategies significantly improve human IL-15 expression. Replacement of native IL-15 signal peptide sequence with that of human IgE leader up-regulated its expression by 5-8 fold demonstrating the negative regulatory feature of the IL-15 leader. Not only did optimized further enhance the expression by 4-15 fold, but even more suprisingly, the less intensive surrogate codon approach as described herein did so as well.

Codon engineering in addition to secretary signal substitution resulted in as much as 40-100 fold increase in IL-15 gene expression in various cell lines tested. The functionality of IL-15 produced from constructs was demonstrated by CTLL2 cell proliferation assay.

Consistent with ‘in vitro’ data, ‘in vivo’ gene expression from the IL-15 constructs according to embodiments of the invention was considerably elevated. Taken together, this data suggest that this combined method represents a novel and unexpected approach for enhancing IL-15 gene expression.

The IgE leader sequence for use in certain embodiments of the invention is provided below.

### IgE Leader Sequence (SEQ ID NO: 11)

ATGGATTGGACTTGGATCTTATTTTTAGTTGCTGCTGCTACTAGAGTTCA
TTCT

The following are the nucleic acid sequences of constructs in accordance with embodiments of the present invention. Leader sequences are indicated by underlining.

Surrogate codon usage HuIL-15 sequence
(SEQ ID NO:12)
ATGCGGATTTCCAAACCTCATCTCAGGTCCATTTCCATCCAGTGCTACCT
CTGTCTCCTCCTCAACTCCCATTTTCTCACGGAAGCTGGCATTCATGTCT
TCATTGTCGGCTGTTTCTCCGCGGGGCTCCCTAAAACGGAAGCCAACTGG
GTGAATGTCATTTCCGATCTCAAAAAAATTGAAGATCTCATTCAATCCAT
GCATATTGATGCGACGCTCTATACGGAATCCGATGTCCACCCCTCCTGCA
AAGTCACCGCGATGAAGTGCTTTCTCCTCGAGCTCCAAGTCATTTCCCTC
GAGTCCGGGGATGCGTCCATTCATGATACGGTCGAAAATCTGATCATCCT
CGCGAACAACTCCCTCTCCTCCAATGGGAATGTCACGGAATCCGGGTGCA
AAGAATGTGAGGAACTGGAGGAAAAAAATATTAAAGAATTTCTCCAGTCC
TTTGTCCATATTGTCCAAATGTTCATCAACACGTCCTAG
IgE leader Human IL-15 sequence (SEQ ID NO:13)
ATGGATTGGACTTGGATCTTATTTTTAGTTGCTGCTGCTACTAGAGTTCA
TTCTAACTGGGTGAATGTAATAAGTGATTTGAAAAAAATTGAAGATCTTA
TTCAATCTATGCATATTGATGCTACTTTATATACGGAAAGTGATGTTCAC
CCCAGTTGCAAAGTAACAGCAATGAAGTGCTTTCTCTTGGAGTTACAAGT
TATTTCACTTGAGTCCGGAGATGCAAGTATTCATGATACAGTAGAAAATC
TGATCATCCTAGCAAACAACAGTTTGTCTTCTAATGGGAATGTAACAGAA
TCTGGATGCAAAGAATGTGAGGAACTGGAGGAAAAAAATATTAAAGAATT
TTTGCAGAGTTTTGTACATATTGTCCAAATGTTCATCAACACTTCTTGA
IgE leader + surrogate codon usage HuIL-15
sequence (SEQ ID NO:14)
ATGGATTGGACGTGGATCCTCTTTCTCGTCGCGGCGGCGACGCGGGTCCA
TTCCAACTGGGTGAATGTCATTTCCGATCTCAAAAAAATTGAAGATCTCA
TTCAATCCATGCATATTGATGCGACGCTCTATACGGAATCCGATGTCCAC
CCCTCCTGCAAAGTCACCGCGATGAAGTGCTTTCTCCTCGAGCTCCAAGT
CATTTCCCTCGAGTCCGGGGATGCGTCCATTCATGATACGGTCGAAAATC
TGATCATCCTCGCGAACAACTCCCTCTCCTCCAATGGGAATGTCACGGAA
TCCGGGTGCAAAGAATGTGAGGAACTGGAGGAAAAAAATATTAAAGAATT
TCTCCAGTCCTTTGTCCATATTGTCCAAATGTTCATCAACACGTCCTAG
IgE leader + optimized HuIL-15 sequence (optimized
alternative 1) (SEQ ID NO:15)
ATGGACTGGACCTGGATCCTGTTCCTGGTGGCCGCCGCCACCCGCGTGCA
CTCCAACTGGGTGAACGTGATCAGCGACCTGAAGAAGATCGAGGACCTGA
TCCAGAGCATGCACATCGACGCCACCCTGTACACCGAGAGCGACGTGCAC
CCCAGCTGCAAGGTGACCGCCATGAAGTGCTTCCTGCTGGAGCTGCAGGT
GATCAGCCTGGAGAGCGGCGACGCCAGCATCCACGACACCGTGGAGAACC
TGATCATCCTGGCCAACAACAGCCTGAGCAGCAACGGCAACGTGACCGAG
AGCGGCTGCAAGGAGTGCGAGGAGCTGGAGGAGAAGAACATCAAGGAGTT
CCTGCAGAGCTTCGTGCACATCGTGCAGATGTTCATCAACACCAGCTAG
IgE leader + Secondary structure optimized HuIL-15
sequence (Optimized Alternative 2) (SEQ ID NO: 16)
ATGGATTGGACCTGGATCCTCTTTCTTGTCGCCGCTGCCACTCGAGTACA
TTCAAACTGGGTAAATGTGATTTCCGACCTTAAAAAAATTGAAGACCTTA
TCCAAAGCATGCACATAGACGCCACCCTTTATACTGAATCCGACGTACAC
CCCTCCTGCAAAGTTACCGCCATGAAATGTTTTCTCCTCGAACTCCAAGT
AATTAGCCTCGAATCCGGAGACGCCTCTATCCACGACACAGTTGAAAACC
TCATAATCCTTGCAAATAACTCTCTTAGCTCAAACGGAAATGTTACTGAA
TCTGGTTGTAAAGAATGCGAAGAACTTGAAGAAAAAAATATAAAAGAATT
TCTGCAATCATTTGTCCACATCGTTCAAATGTTTATCAATACCTCTTAG
The following is the sequence of naturally-
occurring human IL-15 sequence provided herein for
comparative purposes.
Human IL-15 sequence (SEQ ID NO:17)
ATGAGAATTTCGAAACCACATTTGAGAAGTATTTCCATCCAGTGCTACTT
GTGTTTACTTCTAAACAGTCATTTTCTAACTGAAGCTGGCATTCATGTCT
TCATTTTGGGCTGTTTCAGTGCAGGGCTTCCTAAAACAGAAGCCAACTGG
GTGAATGTAATAAGTGATTTGAAAAAAATTGAAGATCTTATTCAATCTAT
GCATATTGATGCTACTTTATATACGGAAAGTGATGTTCACCCCAGTTGCA
AAGTAACAGCAATGAAGTGCTTTCTCTTGGAGTTACAAGTTATTTCACTT
GAGTCTGGAGATGCAAGTATTCATGATACAGTAGAAAATCTGATCATCCT
AGCAAACAACAGTTTGTCTTCTAATGGGAATGTAACAGAATCTGGATGCA
AAGAATGTGAGGAACTGGAGGAAAAAAATATTAAAGAATTTTTGCAGAGT
TTTGTACATATTGTCCAAATGTTCATCAACACTTCTTGA
The following is the nucleic acid sequence for the
O-IL-15-IgE leader plasmid construct
(SEQ ID NO:18):
AAATGGGGGCGCTGAGGTCTGCCTCGTGAAGAAGGTGTTGCTGACTCATA
CCAGGCCTGAATCGCCCCATCATCCAGCCAGAAAGTGAGGGAGCCACGGT
TGATGAGAGCTTTGTTGTAGGTGGACCAGTTGGTGATTTTGAACTTTTGC
TTTGCCACGGAACGGTCTGCGTTGTCGGGAAGATGCGTGATCTGATCCTT
CAACTCAGCAAAAGTTCGATTTATTCAACAAAGCCGCCGTCCCGTCAAGT
CAGCGTAATGCTCTGCCAGTGTTACAACCAATTAACCAATTCTGCGTTCA
AAATGGTATGCGTTTTGACACATCCACTATATATCCGTGTCGTTCTGTCC
ACTCCTGAATCCCATTCCAGAAATTCTCTAGCGATTCCAGAAGTTTCTCA
GAGTCGGAAAGTTGACCAGACATTACGAACTGGCACAGATGGTCATAACC
TGAAGGAAGATCTGATTGCTTAACTGCTTCAGTTAAGACCGACGCGCTCG
TCGTATAACAGATGCGATGATGCAGACCAATCAACATGGCACCTGCCATT
GCTACCTGTACAGTCAAGGATGGTAGAAATGTTGTCGGTCCTTGCACACG
AATATTACGCCATTTGCCTGCATATTCAAACAGCTCTTCTACGATAAGGG
CACAAATCGCATCGTGGAACGTTTGGGCTTCTACCGATTTAGCAGTTTGA
TACACTTTCTCTAAGTATCCACCTGAATCATAAATCGGCAAAATAGAGAA
AAATTGACCATGTGTAAGCGGCCAATCTGATTCCACCTGAGATGCATAAT
CTAGTAGAATCTCTTCGCTATCAAAATTCACTTCCACCTTCCACTCACCG
GTTGTCCATTCATGGCTGAACTCTGCTTCCTCTGTTGACATGACACACAT
CATCTCAATATCCGAATACGGACCATCAGTCTGACGACCAAGAGAGCCAT
AAACACCAATAGCCTTAACATCATCCCCATATTTATCCAATATTCGTTCC
TTAATTTCATGAACAATCTTCATTCTTTCTTCTCTAGTCATTATTATTGG
TCCGTTCATAACACCCCTTGTATTACTGTTTATGTAAGCAGACAGTTTTA
TTGTTCATGATGATATATTTTTATCTTGTGCAATGTAACATCAGAGATTT
TGAGACACAACGTGGCTTTCCCCGGCCCATGACCAAAATCCCTTAACGTG
AGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTG
GCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTT
GGAGCGAACGACCTACACCGAACTGAGATACCTACAGCGTGAGCTATGAG
AAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCT
TTGAGTGAGCTGATACCGCTCGCCGCAGCCGAACGACCGAGCGCAGCGAG
TCAGTGAGCGAGGAAGCGGAAGAGCGCCTGATGCGGTATTTTCTCCTTAC
GCATCTGTGCGGTATTTCACACCGCATATGGTGCACTCTCAGTACAATCT
GCTCTGATGCCGCATAGTTAAGCCAGTATCTGCTCCCTGCTTGTGTGTTG
GAGGTCGCTGAGTAGTGCGCGAGCAAAATTTAAGCTACAACAAGGCAAGG
CTTGACCGACAATTGCATGAAGAATCTGCTTAGGGTTAGGCGTTTTGCGC
TGCTTCGCGATGTACGGGCCAGATATAGCCGCGGCATCGATGATATCCAT
TGCATACGTTGTATCTATATCATAATATGTACATTTATATTGGCTCATGT
CCAATATGACCGCCATGTTGACATTGATTATTGACTAGTTATTAATAGTA
ATCAATTACGGGGTCATTAGTTCATAGCCCATATATGGAGTTCCGCGTTA
CATAACTTACGGTAAATGGCCCGCCTGGCTGACCGCCCAACGACCCCCGC
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTCCGCCCCCTATTGACGTCAAT
GACGGTAAATGGCCCGCCTGGCATTATGCCCAGTACATGACCTTACGGGA
CTTTCCTACTTGGCAGTACATCTACGTATTAGTCATCGCTATTACCATGG
TGATGCGGTTTTGGCAGTACATCAATGGGCGTGGATAGCGGTTTGACTCA
CGGGGATTTCCAAGTCTCCACCCCATTGACGTCAATGGGAGTTTGTTTTG
GCACCAAAATCAACGGGACTTTCCAAAATGTCGTAACAACTCCGCCCCAT
TGACGCAAATGGGCGGTAGGCGTGTACGGTGGGAGGTCTATATAAGCAGA
GCTCGTTTAGTGAACCGTCAGATCGCCTGGAGACGCCATCCACGCTGTTT
TGACCTCCATAGAAGACACCGGGACCGATCCAGCCTCCGCGGGCGCGCGT
CGACCACCATGGACTGGACCTGGATCCTGTTCCTGGTGGCCGCCGCCACC
CGCGTGCACTCCAACTGGGTGAACGTGATCAGCGACCTGAAGAAGATCGA
GGACCTGATCCAGAGCATGCACATCGACGCCACCCTGTACACCGAGAGCG
ACGTGCACCCCAGCTGCAAGGTGACCGCCATGAAGTGCTTCCTGCTGGAG
CTGCAGGTGATCAGCCTGGAGAGCGGCGACGCCAGCATCCACGACACCGT
GGAGAACCTGATCATCCTGGCCAACAACAGCCTGAGCAGCAACGGCAACG
TGACCGAGAGCGGCTGCAAGGAGTGCGAGGAGCTGGAGGAGAAGAACATC
AAGGAGTTCCTGCAGAGCTTCGTGCACATCGTGCAGATGTTCATCAACAC
CAGCTAGTGAGTCGACGGGCGACGCGAAACTTGGGCCCACTCGAGAGGCG
CGCCGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGA
GGATTGGGAAGACAATAGCAGGCATGCTGGGGAATTT
The following is the nucleic acid sequence for the
:LP-IL-15-IgE leader plasmid construct
(SEQ ID NO:19)
AAATGGGGGCGCTGAGGTCTGCCTCGTGAAGAAGGTGTTGCTGACTCATA
CCAGGCCTGAATCGCCCCATCATCCAGCCAGAAAGTGAGGGAGCCACGGT
TGATGAGAGCTTTGTTGTAGGTGGACCAGTTGGTGATTTTGAACTTTTGC
TTTGCCACGGAACGGTCTGCGTTGTCGGGAAGATGCGTGATCTGATCCTT
CAACTCAGCAAAAGTTCGATTTATTCAACAAAGCCGCCGTCCCGTCAAGT
CAGCGTAATGCTCTGCCAGTGTTACAACCAATTAACCAATTCTGCGTTCA
AAATGGTATGCGTTTTGACACATCCACTATATATCCGTGTCGTTCTGTCC
ACTCCTGAATCCCATTCCAGAAATTCTCTAGCGATTCCAGAAGTTTCTCA
GAGTCGGAAAGTTGACCAGACATTACGAACTGGCACAGATGGTCATAACC
TGAAGGAAGATCTGATTGCTTAACTGCTTCAGTTAAGACCGACGCGCTCG
TCGTATAACAGATGCGATGATGCAGACCAATCAACATGGCACCTGCCATT
GCTACCTGTACAGTCAAGGATGGTAGAAATGTTGTCGGTCCTTGCACACG
AATATTACGCCATTTGCCTGCATATTCAAACAGCTCTTCTACGATAAGGG
CACAAATCGCATCGTGGAACGTTTGGGCTTCTACCGATTTAGCAGTTTGA
TACACTTTCTCTAAGTATCCACCTGAATCATAAATCGGCAAAATAGAGAA
AAATTGACCATGTGTAAGCGGCCAATCTGATTCCACCTGAGATGCATAAT
CTAGTAGAATCTCTTCGCTATCAAAATTCACTTCCACCTTCCACTCACCG
GTTGTCCATTCATGGCTGAACTCTGCTTCCTCTGTTGACATGACACACAT
CATCTCAATATCCGAATACGGACCATCAGTCTGACGACCAAGAGAGCCAT
AAACACCAATAGCCTTAACATCATCCCCATATTTATCCAATATTCGTTCC
TTAATTTCATGAACAATCTTCATTCTTTCTTCTCTAGTCATTATTATTGG
TCCGTTCATAACACCCCTTGTATTACTGTTTATGTAAGCAGACAGTTTTA
TTGTTCATGATGATATATTTTTATCTTGTGCAATGTAACATCAGAGATTT
TGAGACACAACGTGGCTTTCCCCGGCCCATGACCAAAATCCCTTAACGTG
AGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTG
GCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTT
GGAGCGAACGACCTACACCGAACTGAGATACCTACAGCGTGAGCTATGAG
AAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCT
TTGAGTGAGCTGATACCGCTCGCCGCAGCCGAACGACCGAGCGCAGCGAG
TCAGTGAGCGAGGAAGCGGAAGAGCGCCTGATGCGGTATTTTCTCCTTAC
GCATCTGTGCGGTATTTCACACCGCATATGGTGCACTCTCAGTACAATCT
GCTCTGATGCCGCATAGTTAAGCCAGTATCTGCTCCCTGCTTGTGTGTTG
GAGGTCGCTGAGTAGTGCGCGAGCAAAATTTAAGCTACAACAAGGCAAGG
CTTGACCGACAATTGCATGAAGAATCTGCTTAGGGTTAGGCGTTTTGCGC
TGCTTCGCGATGTACGGGCCAGATATAGCCGCGGCATCGATGATATCCAT
TGCATACGTTGTATCTATATCATAATATGTACATTTATATTGGCTCATGT
CCAATATGACCGCCATGTTGACATTGATTATTGACTAGTTATTAATAGTA
ATCAATTACGGGGTCATTAGTTCATAGCCCATATATGGAGTTCCGCGTTA
CATAACTTACGGTAAATGGCCCGCCTGGCTGACCGCCCAACGACCCCCGC
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTCCGCCCCCTATTGACGTCAAT
GACGGTAAATGGCCCGCCTGGCATTATGCCCAGTACATGACCTTACGGGA
CTTTCCTACTTGGCAGTACATCTACGTATTAGTCATCGCTATTACCATGG
TGATGCGGTTTTGGCAGTACATCAATGGGCGTGGATAGCGGTTTGACTCA
GGGGGATTTCCAAGTCTCCACCCCATTGACGTCAATGGGAGTTTGTTTTG
GCACCAAAATCAACGGGACTTTCCAAAATGTCGTAACAACTCCGCCCCAT
TGACGCAAATGGGCGGTAGGCGTGTACGGTGGGAGGTCTATATAAGCAGA
GCTCGTTTAGTGAACCGTCAGATCGCCTGGAGACGCCATCCACGCTGTTT
TGACCTCCATAGAAGACACCGGGACCGATCCAGCCTCCGCGGGCGCGCGT
CGACCACCATGGATTGGACGTGGATCCTCTTTCTCGTCGCGGCGGCGACG
CGGGTCCATTCCAACTGGGTGAATGTCATTTCCGATCTCAAAAAAATTGA
AGATCTCATTCAATCCATGCATATTGATGCGACGCTCTATACGGAATCCG
ATGTCCACCCCTCCTGCAAAGTCACCGCGATGAAGTGCTTTCTCCTCGAG
CTCCAAGTCATTTCCCTCGAGTCCGGGGATGCGTCCATTCATGATACGGT
CGAAAATCTGATCATCCTCGCGAACAACTCCCTCTCCTCCAATGGGAATG
TCACGGAATCCGGGTGCAAAGAATGTGAGGAACTGGAGGAAAAAAATATT
AAAGAATTTCTCCAGTCCTTTGTCCATATTGTCCAAATGTTCATCAACAC
GTCCTAGTGAGTCGACGGGCGACGCGAAACTTGGGCCCACTCGAGAGGCG
CGCCGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGA
GGATTGGGAAGACAATAGCAGGCATGCTGGGGAATTT
The following is the nucleic acid sequence for the
BH-IL-15-IgE leader plasmid construct
(SEQ ID NO:20)
AAATGGGGGCGCTGAGGTCTGCCTCGTGAAGAAGGTGTTGCTGACTCATA
CCAGGCCTGAATCGCCCCATCATCCAGCCAGAAAGTGAGGGAGCCACGGT
TGATGAGAGCTTTGTTGTAGGTGGACCAGTTGGTGATTTTGAACTTTTGC
TTTGCCACGGAACGGTCTGCGTTGTCGGGAAGATGCGTGATCTGATCCTT
CAACTCAGCAAAAGTTCGATTTATTCAACAAAGCCGCCGTCCCGTCAAGT
CAGCGTAATGCTCTGCCAGTGTTACAACCAATTAACCAATTCTGCGTTCA
AAATGGTATGCGTTTTGACACATCCACTATATATCCGTGTCGTTCTGTCC
ACTCCTGAATCCCATTCCAGAAATTCTCTAGCGATTCCAGAAGTTTCTCA
GAGTCGGAAAGTTGACCAGACATTACGAACTGGCACAGATGGTCATAACC
TGAAGGAAGATCTGATTGCTTAACTGCTTCAGTTAAGACCGACGCGCTCG
TCGTATAACAGATGCGATGATGCAGACCAATCAACATGGCACCTGCCATT
GCTACCTGTACAGTCAAGGATGGTAGAAATGTTGTCGGTCCTTGCACACG
AATATTACGCCATTTGCCTGCATATTCAAACAGCTCTTCTACGATAAGGG
CACAAATCGCATCGTGGAACGTTTGGGCTTCTACCGATTTAGCAGTTTGA
TACACTTTCTCTAAGTATCCACCTGAATCATAAATCGGCAAAATAGAGAA
AAATTGACCATGTGTAAGCGGCCAATCTGATTCCACCTGAGATGCATAAT
CTAGTAGAATCTCTTCGCTATCAAAATTCACTTCCACCTTCCACTCACCG
GTTGTCCATTCATGGCTGAACTCTGCTTCCTCTGTTGACATGACACACAT
CATCTCAATATCCGAATACGGACCATCAGTCTGACGACCAAGAGAGCCAT
AAACACCAATAGCCTTAACATCATCCCCATATTTATCCAATATTCGTTCC
TTAATTTCATGAACAATCTTCATTCTTTCTTCTCTAGTCATTATTATTGG
TCCGTTCATAACACCCCTTGTATTACTGTTTATGTAAGCAGACAGTTTTA
TTGTTCATGATGATATATTTTTATCTTGTGCAATGTAACATCAGAGATTT
TGAGACACAACGTGGCTTTCCCCGGCCCATGACCAAAATCCCTTAACGTG
AGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTG
GCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTT
GGAGCGAACGACCTACACCGAACTGAGATACCTACAGCGTGAGCTATGAG
AAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCT
TTGAGTGAGCTGATACCGCTCGCCGCAGCCGAACGACCGAGCGCAGCGAG
TCAGTGAGCGAGGAAGCGGAAGAGCGCCTGATGCGGTATTTTCTCCTTAC
GCATCTGTGCGGTATTTCACACCGCATATGGTGCACTCTCAGTACAATCT
GCTCTGATGCCGCATAGTTAAGCCAGTATCTGCTCCCTGCTTGTGTGTTG
GAGGTCGCTGAGTAGTGCGCGAGCAAAATTTAAGCTACAACAAGGCAAGG
CTTGACCGACAATTGCATGAAGAATCTGCTTAGGGTTAGGCGTTTTGCGC
TGCTTCGCGATGTACGGGCCAGATATAGCCGCGGCATCGATGATATCCAT
TGCATACGTTGTATCTATATCATAATATGTACATTTATATTGGCTCATGT
CCAATATGACCGCCATGTTGACATTGATTATTGACTAGTTATTAATAGTA
ATCAATTACGGGGTCATTAGTTCATAGCCCATATATGGAGTTCCGCGTTA
CATAACTTACGGTAAATGGCCCGCCTGGCTGACCGCCCAACGACCCCCGC
CCATTGACGTCAATAATGACGTATGTTCCCATAGTAACGCCAATAGGGAC
TTTCCATTGACGTCAATGGGTGGAGTATTTACGGTAAACTGCCCACTTGG
CAGTACATCAAGTGTATCATATGCCAAGTCCGCCCCCTATTGACGTCAAT
GACGGTAAATGGCCCGCCTGGCATTATGCCCAGTACATGACCTTACGGGA
CTTTCCTACTTGGCAGTACATCTACGTATTAGTCATCGCTATTACCATGG
TGATGCGGTTTTGGCAGTACATCAATGGGCGTGGATAGCGGTTTGACTCA
CGGGGATTTCCAAGTCTCCACCCCATTGACGTCAATGGGAGTTTGTTTTG
GCACCAAAATCAACGGGACTTTCCAAAATGTCGTAACAACTCCGCCCCAT
TGACGCAAATGGGCGGTAGGCGTGTACGGTGGGAGGTCTATATAAGCAGA
GCTCGTTTAGTGAACCGTCAGATCGCCTGGAGACGCCATCCACGCTGTTT
TGACCTCCATAGAAGACACCGGGACCGATCCAGCCTCCGCGGGCGCGCGT
CGACCACCATGGATTGGACCTGGATCCTCTTTCTTGTCGCCGCTGCCACT
CGAGTACATTCAAACTGGGTAAATGTGATTTCCGACCTTAAAAAAATTGA
AGACCTTATCCAAAGCATGCACATAGACGCCACCCTTTATACTGAATCCG
ACGTACACCCCTCCTGCAAAGTTACCGCCATGAAATGTTTTCTCCTCGAA
CTCCAAGTAATTAGCCTCGAATCCGGAGACGCCTCTATCCACGACACAGT
TGAAAACCTCATAATCCTTGCAAATAACTCTCTTAGCTCAAACGGAAATG
TTACTGAATCTGGTTGTAAAGAATGCGAAGAACTTGAAGAAAAAAATATA
AAAGAATTTCTGCAATCATTTGTCCACATCGTTCAAATGTTTATCAATAC
CTCTTAGTGAGTCGACGGGCGACGCGAAACTTGGGCCCACTCGAGAGGCG
CGCCGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGT
AGGTGTCATTCTATTCTGGGGGGTGGGGTGGGGCAGGACAGCAAGGGGG
AGGATTGGGAAGACAATAGCAGGCATGCTGGGGAATTT

The invention now being fully described, it will be apparent to one of ordinary skill in the art that many changes and modifications can be made thereto without departing from the spirit or scope of the invention as set forth herein. The foregoing describes the preferred embodiments of the present invention along with a number of possible alternatives. These embodiments, however, are merely for example and the invention is not restricted thereto.

1-397. (canceled)

398. A modified polynucleotide comprising: a nucleic acid sequence comprising one or more surrogate codons in place of a corresponding naturally-occurring codon having adenine (A), thymine (T), or uracil (U) in the wobble position; wherein the surrogate codon encodes the same amino acid as the naturally-occurring codon.

399. The modified polynucleotide of claim 398, wherein the surrogate codons encode any of the amino acids alanine, arginine, leucine, proline, glutamic acid, glycine, isoleucine, serine, threonine, or valine.

400. The modified polynucleotide of claim 399, wherein the surrogate codons comprise cytosine (C) or guanine (G) at the wobble position.

401. The modified polynucleotide of claim 399, wherein the surrogate codon encoding alanine is GCG, encoding arginine is CGG or AGG, encoding leucine is CTC, encoding proline is CCT or CCG, encoding glutamic acid is GAG, encoding glycine is GGG, encoding isoleucine is ATT, encoding serine is TCC, encoding threonine is ACG, and encoding valine is GTC.

402. The modified polynucleotide of claim 398, additionally comprising a non-native leader sequence.

403. The modified polynucleotide of claim 398, additionally comprising a human non-native leader sequence.

404. The modified polynucleotide of claim 398, additionally comprising an immunoglobulin leader sequence.

405. The modified polynucleotide of claim 398, additionally comprising (a) an IgE leader sequence or (b) a leader sequence that hybridizes to an IgE leader sequence under stringent conditions.

406. The modified polynucleotide of claim 398, additionally comprising a leader sequence comprising SEQ ID NO: 11.

407. The modified polynucleotide of claim 406, wherein the leader sequence has at least 90% sequence identity to the nucleic acid sequence of SEQ ID NO: 11.

408. The modified polynucleotide of claim 406, wherein the leader sequence has at least 95% sequence identity to the nucleic acid sequence of SEQ ID NO: 11.

409. The modified polynucleotide of claim 406, wherein the leader sequence is the nucleic acid sequence of SEQ ID NO: 11.

410. The modified polynucleotide of claim 398, wherein the modified polynucleotide encodes a viral, bacterial, protist, fungal, plant, or animal polypeptide.

411. The modified polynucleotide of claim 410, wherein the modified polynucleotide encodes a mammalian polypeptide.

412. The modified polynucleotide of claim 410. wherein the viral polypeptide is an HPV16 polypeptide or an HIV-1 polypeptide.

413. The modified polynucleotide of claim 398, wherein the modified polynucleotide comprises the open reading frame (ORF) for the HPV16 E7 gene, HIV-1 gag gene, or gp160 envelope gene.

414. The modified polynucleotide of claim 398, wherein the surrogate codons are a randomized selection of at least about 10% of the codons in said modified polynucleotide that encode for any of the amino acids alanine, arginine, leucine, proline, glutamic acid, glycine, isoleucine, serine, threonine and valine.

415. The modified polynucleotide of claim 398, wherein the surrogate codons are a randomized selection of at least about 50% of the codons in said modified polynucleotide that encode for any of the amino acids alanine, arginine, leucine, proline, glycine, isoleucine, serine, threonine and valine.

416. The modified polynucleotide of claim 398, wherein the surrogate codons are a randomized selection of at least about 90% of the codons in said modified polynucleotide that encode for any of the amino acids alanine, arginine, leucine, proline, glycine, isoleucine, serine, threonine and valine.

417. The modified polynucleotide of claim 398, wherein the modified polynucleotide is a DNA molecule.

418. The modified polynucleotide of claim 398, wherein the modified polynucleotide is an RNA molecule.

419. The modified polynucleotide of claim 398, wherein the nucleic acid sequence comprises any of:
(a) the nucleic acid sequence encoding any of SEQ ID NOS: 2,4, or 6;
(b) an immunogenic encoding portion of SEQ ID NOS: 2, 4 or 6; or
(c) a nucleic acid sequence that hybridizes under stringent conditions to the nucleic acid sequence encoding any of SEQ ID NOS: 2,4, or 6.

420. The modified polynucleotide of claim 398, wherein the nucleic acid sequence comprises any of:
(a) a nucleic acid sequence having at least about 70% sequence identity to the nucleic acid sequence of SEQ ID NO: 14; or
(b) a nucleic acid sequence that hybridizes to SEQ ID NO: 14 under stringent conditions.

421. The modified polynucleotide of claim 398, wherein the nucleic acid sequence comprises any of:
(a) the nucleic acid sequence encoding any of SEQ ID NOS: 12-16;
(b) an immunogenic encoding portion of SEQ ID NOS: 12-16; or
(c) a nucleic acid sequence that hybridizes under stringent conditions to the nucleic acid sequence encoding any of SEQ ID NOS: 12-16.

422. The modified polynucleotide of claim 398, wherein the modified polynucleotide sequence has at least 90% sequence identity to the nucleic acid sequence of any of SEQ ID NOS: 12-16.

423. The modified polynucleotide of claim 398, wherein the modified polynucleotide sequence has at least 95% sequence identity to the nucleic acid sequence of any of SEQ ID NOS: 12-16.

424. A composition comprising the modified polynucleotide of claim 398 and a pharmaceutically acceptable vector.

425. A composition comprising the nucleic acid sequence of any of SEQ ID NOS: 1, 3, 5, 12, 13, 14, 15, or 16.

426. A method for preparing a polynucleotide that provides enhanced expression of a gene comprising:
assembling oligonucleotides comprising surrogate codons to form a modified polynucleotide comprising one or more surrogate codons in place of a corresponding naturally-occurring codon having adenine (A), thymine (T), or uracil (U) in the wobble position; wherein the surrogate codon encodes the same amino acid as the naturally-occurring codon.

427. The method of claim 426, wherein the surrogate codon encodes any of the amino acids alanine, arginine, leucine, proline, glutamic acid, glycine, isoleucine, serine, threonine and valine.

428. The method of claim 426, wherein the surrogate codons comprises cytosine (C) or guanine (G) at the wobble position.

429. The method of claim 426, wherein the surrogate codon encoding alanine is GCG, encoding arginine is CGG or AGG, encoding leucine is CTC, encoding proline is CCT or CCG, encoding glutamic acid is GAG, encoding glycine is GGG, encoding iso is ATT, encoding serine is TCC, encoding threonine is ACG, and encoding valine is GTC.

430. The method of claim 426, additionally comprising adding a non-native leader sequence to the modified polynucleotide.

431. The method of claim 426, additionally comprising adding a human non-native leader sequence to the modified polynucleotide.

432. The method of claim 426, additionally comprising adding an immunoglobulin leader sequence to the modified polynucleotide.

433. The method of claim 432, wherein the immunoglobulin leader sequence is: (a) an IgE leader sequence or (b) a leader sequence that hybridizes to an IgE leader sequence under stringent conditions.

434. The method of claim 433, wherein the immunoglobulin leader sequence is an IgE leader sequence.

435. The method of claim 432, additionally comprising adding to the modified polynucleotide a leader sequence comprising SEQ ID NO: 11.

436. The method of claim 432, additionally comprising adding to the modified polynucleotide a leader sequence having at least 95% sequence identity to the nucleic acid sequence of SEQ ID NO: 11.

437. A method for preparing a modified polynucleotide that provides enhanced expression of a polynucleotide sequence comprising:
providing a polynucleotide sequence having a plurality of codons having the nucleotides adenine (A) or uracil (U) or thymine (T) at the wobble position; substituting one or more codons having the nucleotides adenine (A) or uracil (U) or thymine (T) at the wobble position with a surrogate codon having the nucleotides cytosine (C) or guanine (G) at the wobble position; wherein the surrogate codon encodes the same amino acid as the codons having the nucleotides adenine (A) or uracil (U) or thymine (T) at the wobble position; and attaching a leader sequence to the polynucleotide sequence, wherein the leader sequence is a non-native leader sequence to the polynucleotide sequence.

438. A method for enhancing expression of a gene comprising:
expressing in vivo or in vitro the modified polynucleotide modified polynucleotide comprising: a nucleic acid sequence comprising one or more surrogate codons in place of a corresponding naturally-occurring codon having adenine (A), thymine (T), or uracil (U) in the wobble position; wherein the surrogate codon encodes the same amino acid as the naturally-occurring codon.

439. A method of preventing or treating a disease in a mammal comprising: administering to the mammal an effective amount of a composition comprising a nucleic acid sequence comprising one or more surrogate codons in place of a corresponding naturally-occurring codon having adenine (A), thymine (T), or uracil (U) in the wobble position; wherein the surrogate codon encodes the same amino acid as the naturally-occurring codon.

440. The method of claim 439, wherein the composition is administered parenterally, mucosally, subcutaneously, or intramuscularly.


SEQUENCE LISTING (20 sequences)

SEQ ID NO: 1 - DNA, 300 bp, Human papillomavirus
  CDS (1)..(294)
atgcatggggatacgcctacgctccatgaatatatgctcgatctccaacctgagacgacggatctctactgttatgagcaactcaatgacagctccgaggaggaggatgaaattgatgggcctgcggggcaagcggaacctgaccgggcccattacaatattgtcaccttttgttgcaagtgtgactccacgctccggctctgcgtccaaagcacgcacgtcgacattcggacgctcgaagacctgctcatgggcacgctcgggattgtgtgccccatctgttcccagaaaccttaatag

SEQ ID NO: 2 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - DNA, 1092 bp, Human immunodeficiency virus type 1
  CDS (1)..(1089)
atgggggcgcgggcgtccgtcctctccgggggggagctcgatcggtgggagaaaattcggctccggccgggggggaagaaaaaatataaactcaaacatattgtctgggcgtcccgggagctcgagcggttcgcggtcaatccggggctgctcgagacgtccgagggctgtcggcaaattctcgggcagctccaaccgtccctccagacggggtccgaggagctccggtccctctataatacggtcgcgacgctctattgtgtccatcaacggattgagattaaagacacgaaggaggcgctcgacaagattgaggaggagcaaaacaaatccaagaaaaaagcgcagcaagcggcggcggacacggggcactccaatcaggtctcccaaaattacccgattgtccagaacattcaggggcaaatggtccatcaggcgatttccccgcggacgctcaatgcgtgggtcaaagtcgtcgaggagaaggcgttctccccggaggtcattccgatgttttcagcgctctccgagggggcgacgccgcaagatctcaacacgatgctcaacacggtcggggggcatcaagcggcgatgcaaatgctcaaagagacgattaatgaggaggcggcggagtgggatcgggtccatccggtccatgcggggccgattgcgccggggcagatgcgggagccgcgggggtccgacattgcggggacgacgtccacgctccaggagcaaattgggtggatgacgaataatccgccgattccggtcggggagatttataaacggtggattattctcgggctcaataaaattgtccggatgtattccccgacgtccattctcgacattcggcaagggcccaaggagccgtttcgggactatgtagaccggttctataaaacgctccgggcggagcaagcgtcccaggaggtcaaaaattggatgacggagacgctcctcgtccaaaatgcgaacccggattgtaagacgattctcaaagcgctcgggccggcggctacgctcgaggagatgatgacggcgtgtcagggggtcggggggccggggcataaggcgcgggtcctctaa

SEQ ID NO: 4 - protein, 363 aa, Human immunodeficiency virus type 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 5 - DNA, 2568 bp, Human immunodeficiency virus type 1
  CDS (1)..(2562)
atgcgggcgaaggagatgcggaagtcctgtcagcacctccggaaatgggggattctcctctttggggtcctcatgatttgttccgcggaggagaagctctgggtcacggtctattatggggtcccggtctggaaagaggcgacgacgacgctcttttgtgcgtccgatgcgaaggcgcatcatgcggaggcgcataatgtctgggcgacgcatgcgtgtgtcccgacggacccgaacccgcaagaggtcattctcgagaatgtcacggagaaatataacatgtggaaaaataacatggtagaccagatgcatgaggatattatttccctctgggatcaatccctcaagccgtgtgtcaaactcacgccgctctgtgtcacgctcaattgcacgaatgcgacgtatacgaattccgactccaagaattccactagtaattcctccctcgaggactccgggaaaggggacatgaactgctccttcgatgtcacgacgtccattgataaaaagaagaagacggagtatgcgatttttgataaactcgatgtcatgaatattgggaatgggcggtatacgctcctcaattgtaacaggtccgtcattacgcaggcgtgtccgaagatgtcctttgagccgattccgattcattattgtacgccggcggggtatgcgattctcaagtgtaatgataataagttcaatgggacggggccgtgtacgaatgtctccacgattcaatgtacgcatgggattaagccggtcgtctccacgcaactcctcctcaatggatccctcgcggaggggggggaggtcattattcggtccgagaatctcacggacaatgcgaaaacgattattgtccagctcaaggagccggtcgagattaattgtacgcggccgaacaacaatacgcggaaatccattcatatggggccgggggcggcgttttatgcgcggggggaggtcattggggatattcggcaagcgcattgcaacatttcccgggggcggtggaatgacacgctcaaacagattgcgaaaaaactccgggagcaatttaataaaacgatttccctcaaccaatcctccgggggggacctcgagattgtcatgcacacgtttaattgtgggggggagtttttctactgtaatacgacgcagctctttaattccacgtggaatgagaatgatacgacgtggaataatacggcggggtccaataacaatgagacgattacgctcccgtgtcggattaaacaaattattaaccggtggcaggaggtcgggaaagcgatgtatgcgccgccgatttccgggccgattaattgtctctccaatattacggggctcctcctcacgcgtgatgggggggacaataataatacgattgagacgttccggccgggggggggggatatgcgggacaattggcggtccgagctctataaatataaagtcgtccggattgagccgctcgggattgcgccgacgaaggcgaagcggcgggtcgtccaacgggagaaacgggcggtcgggattggggcgatgttcctcgggttcctcggggcggcggggtccacgatgggggcggcgtccgtcacgctcacggtccaggcgcggctcctcctctccgggattgtccaacagcaaaacaatctcctcggggcgattgaggcgcaacagcatctcctccaactcacggtctgggggattaagcagctccaggcgcgggtcctcgcgatggagcggtacctcaaggatcaacagctcctcgggatttgggggtgctccgggaaactcatttgcacgacgaatgtcccgtggaatgcgtcctggtccaataaatccctcgacaagatttggcataacatgacgtggatggagtgggaccgggagattgacaattacacgaaactcatttacacgctcattgaggcgtcccagattcagcaggagaagaatgagcaagagctcctcgagctcgattcctgggcgtccctctggtcctggtttgacatttccaaatggctctggtatattggggtcttcattattgtcattggggggctcgtcgggctcaaaattgtctttgcggtcctctccattgtcaatcgggtccggcaggggtactccccgctctcctttcagacgcggctcccggcgccgcgggggccggaccggccggaggggattgaggagggggggggggagcgggaccgggacagatctgatcaactcgtcacggggttcctcgcgctcatttgggacgatctccggtccctctgcctcttctcctaccaccggctccgggacctcctcctcattgtcgcgcggattgtcgagctcctcgggcggcgggggtgggaggcgctcaagtattggtggaatctcctccaatattggattcaggagctcaagaattccgcggtctccctcctcaacgcgacggcgattgcggtcgcggaggggacggatcggattattgaggtcgtccaacggattgggcgggcgattctccacattccgcggcggattccgcagggggtccagcgggcgctcctctaatga

SEQ ID NO: 6 - protein, 854 aa, Human immunodeficiency virus type 1
MRAKEMRKSCQHLRKWGILLFGVLMICSAEEKLWVTVYYGVPVWKEATTTLFCASDAKAHHAEAHNVWATHACVPTDPNPQEVILENVTEKYNMWKNNMVDQMHEDIISLWDQSLKPCVKLTPLCVTLNCTNATYTNSDSKNSTSNSSLEDSGKGDMNCSFDVTTSIDKKKKTEYAIFDKLDVMNIGNGRYTLLNCNRSVITQACPKMSFEPIPIHYCTPAGYAILKCNDNKFNGTGPCTNVSTIQCTHGIKPVVSTQLLLNGSLAEGGEVIIRSENLTDNAKTIIVQLKEPVEINCTRPNNNTRKSIHMGPGAAFYARGEVIGDIRQAHCNISRGRWNDTLKQIAKKLREQFNKTISLNQSSGGDLEIVMHTFNCGGEFFYCNTTQLFNSTWNENDTTWNNTAGSNNNETITLPCRIKQIINRWQEVGKAMYAPPISGPINCLSNITGLLLTRDGGDNNNTIETFRPGGGDMRDNWRSELYKYKVVRIEPLGIAPTKAKRRVVQREKRAVGIGAMFLGFLGAAGSTMGAASVTLTVQARLLLSGIVQQQNNLLGAIEAQQHLLQLTVWGIKQLQARVLAMERYLKDQQLLGIWGCSGKLICTTNVPWNASWSNKSLDKIWHNMTWMEWDREIDNYTKLIYTLIEASQIQQEKNEQELLELDSWASLWSWFDISKWLWYIGVFIIVIGGLVGLKIVFAVLSIVNRVRQGYSPLSFQTRLPAPRGPDRPEGIEEGGGERDRDRSDQLVTGFLALIWDDLRSLCLFSYHRLRDLLLIVARIVELLGRRGWEALKYWWNLLQYWIQELKNSAVSLLNATAIAVAEGTDRIIEVVQRIGRAILHIPRRIPQGVQRALL

SEQ ID NO: 7 - DNA, 4418 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttgtaggtttaaactaagccgaattctgcagatcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 8 - DNA, 4396 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgccaccatgggggcgcgggcgtccgtcctctccgggggggagctcgatcggtgggagaaaattcggctccggccgggggggaagaaaaaatataaactcaaacatattgtctgggcgtcccgggagctcgagcggttcgcggtcaatccggggctgctcgagacgtccgagggctgtgcgcaaattctcgggcagctccaaccgtccctccagacggggtccgaggagctccggtccctctataatacggtcgcgacgctctattgtgtccatcaacggattgagattaaagacacgaaggaggcgctcgacaagattgaggaggagcaaaacaaatccaagaaaaaagcgcagcaagcggcggcggacacggggcactccaatcaggtctcccaaaattacccgattgtccagaacattcaggggcaaatggtccatcaggcgatttccccgcggacgctcaatgcgtgggtcaaagtcgtcgaggagaaggcgttctccccggaggtcattccgatgttttcagcgctctccgagggggcgacgccgcaagatctcaacacgatgctcaacacggtcggggggcatcaagcggcgatgcaaatgctcaaagagacgattaatgaggaggcggcggagtgggatcgggtccatccggtccatgcggggccgattgcgccggggcagatgcgggagccgcgggggtccgacattgcggggacgacgtccacgctccaggagcaaattgggtggatgacgaataatccgccgattccggtcggggagatttataaacggtggattattctcgggctcaataaaattgtccggatgtattccccgacgtccattctcgacattcggcaagggccgaaggagccgtttcgggactatgtagaccggttctataaaacgctccgggcggagcaagcgtcccaggaggtcaaaaattggatgacggagacgctcctcgtccaaaatgcgaacccggattgtaagacgattctcaaagcgctcgggccggcggctacgctcgaggagatgatgacggcgtgtcagggggtcggggggccggggcataaggcgcgggtcctctaatgaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 9 - DNA, 5869 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgccaccatgcgggcgaaggagatgcggaagtcctgtcagcacctccggaaatgggggattctcctctttggggtcctcatgatttgttccgcggaggagaagctctgggtcacggtctattatggggtcccggtctggaaagaggcgacgacgacgctcttttgtgcgtccgatgcgaaggcgcatcatgcggaggcgcataatgtctgggcgacgcatgcgtgtgtcccgacggacccgaacccgcaagaggtcattctcgagaatgtcacggagaaatataacatgtggaaaaataacatggtagaccagatgcatgaggatattatttccctctgggatcaatccctcaagccgtgtgtcaaactcacgccgctctgtgtcacgctcaattgcacgaatgcgacgtatacgaattccgactccaagaattccactagtaattcctccctcgaggactccgggaaaggggacatgaactgctccttcgatgtcacgacgtccattgataaaaagaagaagacggagtatgcgatttttgataaactcgatgtcatgaatattgggaatgggcggtatacgctcctcaattgtaacacgtccgtcattacgcaggcgtgtccgaagatgtcctttgagccgattccgattcattattgtacgccggcggggtatgcgattctcaagtgtaatgataataagttcaatgggacggggccgtgtacgaatgtctccacgattcaatgtacgcatgggattaagccggtcgtctccacgcaactcctcctcaatggatccctcgcggaggggggggaggtcattattcggtccgagaatctcacggacaatgcgaaaacgattattgtccagctcaaggagccggtcgagattaattgtacgcggccgaacaacaatacgcggaaatccattcatatggggccgggggcggcgttttatgcgcggggggaggtcattggggatattcggcaagcgcattgcaacatttcccgggggcggtggaatgacacgctcaaacagattgcgaaaaaactccgggagcaatttaataaaacgatttccctcaaccaatcctccgggggggacctcgagattgtcatgcacacgtttaattgtgggggggagtttttctactgtaatacgacgcagctctttaattccacgtggaatgagaatgatacgacgtggaataatacggcggggtccaataacaatgagacgattacgctcccgtgtcggattaaacaaattattaaccggtggcaggaggtcgggaaagcgatgtatgcgccgccgatttccgggccgattaattgtctctccaatattacggggctcctcctcacgcgtgatgggggggacaacaataatacgattgagacgttccggccgggggggggggatatgcgggacaattggcggtccgagctctataaatataaagtcgtccggattgagccgctcgggattgcgccgacgaaggcgaagcggcgggtcgtccaacgggagaaacgggcggtcgggattggggcgatgttcctcgggttcctcggggcggcggggtccacgatgggggcggcgtccgtcacgctcacggtccaggcgcggctcctcctctccgggattgtccaacagcaaaacaatctcctccgggcgattgaggcgcaacagcatctcctccaactcacggtctgggggattaagcagctccaggcgcgggtcctcgcgatggagcggtacctcaaggatcaacagctcctcgggatttgggggtgctccgggaaactcatttgcacgacgaatgtcccgtggaatgcgtcctggtccaataaatccctcgacaagatttggcataacatgacgtggatggagtgggaccgggagattgacaattacacgaaactcatttacacgctcattgaggcgtcccagattcagcaggagaagaatgagcaagagctcctcgagctcgattcctgggcgtccctctggtcctggtttgacatttccaaatggctctggtatattggggtcttcattattgtcattggggggctcgtcgggctcaaaattgtctttgcggtcctctccattgtcaatcgggtccggcaggggtactccccgctctcctttcagacgcggctcccggcgccgcgggggccggaccggccggaggggattgaggagggggggggggagcgggaccgggacagatctgatcaactcgtcacggggttcctcgcgctcatttgggacgatctccggtccctctgcctcttctcctaccaccggctccgggacctcctcctcattgtcgcgcggattgtcgagctcctcgggcggcgggggtgggaggcgctcaagtattggtggaatctcctccaatattggattcaggagctcaagaattccgcggtctccctcctcaacgcgacggcgattgcggtcgcggaggggacggatcggattattgaggtcgtccaacggattgggcgggcgattctccacattccgcggcggattcggcaggggctcgagcgggcgctcctctaatgaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 10 - DNA, 5946 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatcgcggctatctgaggggactagggtgtgtttaggcgaaaagcggggcttcggttgtacgcggttaggagtcccctcaccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgccaccatgagagcgaaggagatgaggaagagttgtcagcacttgaggaaatggggcatcttgctctttggagtgttgatgatctgtagtgctgaagaaaagttgtgggtcacagtctattatggggtacctgtgtggaaagaagcaaccaccactctattttgtgcatcagatgctaaggcacatcatgcagaggcacataatgtttgggccacacatgcctgtgtacccacagaccctaacccacaagaagtaatattggaaaatgtgacagaaaaatataacatgtggaaaaataacatggtagaccagatgcatgaggatataatcagtttatgggatcaaagcctaaagccatgtgtaaaattaaccccactctgtgttactttaaattgcactaatgcgacgtatactaatagtgacagtaagaatagtaccagtaatagtagtttggaagacagtgggaaaggagacatgaactgctctttcgatgtcaccacaagcatagataaaaagaagaagacagaatatgcaatttttgataaacttgatgtaatgaatataggtaatggaagatatacattactaaattgtaacacctcagtcattacacaggcctgtccaaagatgtcctttgaaccaattcccatacattattgtaccccggctggttatgcgattctaaagtgtaatgataataagttcaatggaacaggaccatgtacaaatgtcagcacaatacaatgtacacatggaattaagccagtagtgtcaactcaactgctgttaaatggcagtctagcagaaggaggagaggtaataattagatctgaaaatctcacagacaatgctaaaaccataatagtacagctcaaggaacctgtagaaatcaattgtacaagacccaacaacaatacaagaaaaagtatacatatgggaccaggagcagcattttatgcaagaggagaagtaataggagatataagacaagcacattgcaacattagtagaggaagatggaatgacactttaaaacagatagctaaaaaattaagagaacaatttaataaaacaataagccttaaccaatcctcaggaggggacctagaaattgtaatgcacacttttaattgtggaggggaatttttctactgtaatacaacacagctgtttaatagtacttggaatgagaatgatactacctggaataatacagcagggtcaaataacaatgaaactatcacactcccatgtagaataaaacaaattataaacaggtggcaggaagtaggaaaagcaatgtatgcccctcccatcagtggaccaattaattgtttatcaaatatcacagggctattattaacaagagatggtggtgacaacaataatacaatagagaccttcagacctggaggaggagatatgagggacaattggagaagtgaattatataaatataaagtagtaagaattgagccattaggaatagcacccaccaaggcaaagagaagagtggtgcaaagagaaaaaagagcagtgggaataggagctatgttccttgggttcttgggagcagcaggaagcactatgggcgcagcgtcagtgacgctgacggtacaggccagactattattgtctggtatagtgcaacagcaaaacaatttgctgagagctatcgaggcgcaacagcatctgttgcaactcacagtctggggcatcaagcagctccaggctagagtcctggctatggaaagatacctaaaggatcaacagctcctagggatttggggttgctctggaaaactcatttgcaccactaatgtgccttggaatgctagttggagtaataaatctctggacaagatttggcataacatgacctggatggagtgggacagagaaattgacaattacacaaaattaatatacaccttaattgaagcatcgcagatccagcaggaaaagaatgaacaagaattattggaattggatagttgggcaagtttgtggagttggtttgacatctcaaaatggctgtggtatataggagtattcataatagtaataggaggtttagtaggtttaaaaatagtttttgctgtactttctatagtaaatagagttaggcagggatactcaccattatcatttcagacccgcctcccagccccgcggggacccgacaggcccgaaggaatcgaagaaggaggtggagagagagacagagacagatccgatcaattagtgactggattcttagcactcatctgggacgatctgcggagcctgtgcctcttcagctaccaccgcttgagagacttactcttgattgtagcgaggattgtggaacttctgggacgcagggggtgggaagccctgaagtattggtggaatctcctgcaatattggattcaggaactaaagaatagtgctgttagtttgcttaacgccacagctatagcagtagccgaggggacagataggattatagaagtagtacaaaggattggtagagctattctccacatacctagaagaataagacagggcttagaaagggctttgctataatagggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 11 - DNA, 54 bp, Artificial Sequence
  Description of Artificial Sequence Synthetic oligonucleotide
atggattggacttggatcttatttttagttgctgctgctactagagttcattct

SEQ ID NO: 12 - DNA, 489 bp, Artificial Sequence
  Description of Artificial Sequence Synthetic construct
atgcggatttccaaacctcatctcaggtccatttccatccagtgctacctctgtctcctcctcaactcccattttctcacggaagctggcattcatgtcttcattgtcggctgtttctccgcggggctccctaaaacggaagccaactgggtgaatgtcatttccgatctcaaaaaaattgaagatctcattcaatccatgcatattgatgcgacgctctatacggaatccgatgtccacccctcctgcaaagtcaccgcgatgaagtgctttctcctcgagctccaagtcatttccctcgagtccggggatgcgtccattcatgatacggtcgaaaatctgatcatcctcgcgaacaactccctctcctccaatgggaatgtcacggaatccgggtgcaaagaatgtgaggaactggaggaaaaaaatattaaagaatttctccagtcctttgtccatattgtccaaatgttcatcaacacgtcctag

SEQ ID NO: 13 - DNA, 399 bp, Artificial Sequence
  Description of Artificial Sequence Synthetic construct
atggattggacttggatcttatttttagttgctgctgctactagagttcattctaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 14 - DNA, 399 bp, Artificial Sequence
  Description of Artificial Sequence Synthetic construct
atggattggacgtggatcctctttctcgtcgcggcggcgacgcgggtccattccaactgggtgaatgtcatttccgatctcaaaaaaattgaagatctcattcaatccatgcatattgatgcgacgctctatacggaatccgatgtccacccctcctgcaaagtcaccgcgatgaagtgctttctcctcgagctccaagtcatttccctcgagtccggggatgcgtccattcatgatacggtcgaaaatctgatcatcctcgcgaacaactccctctcctccaatgggaatgtcacggaatccgggtgcaaagaatgtgaggaactggaggaaaaaaatattaaagaatttctccagtcctttgtccatattgtccaaatgttcatcaacacgtcctag

SEQ ID NO: 15 - DNA, 399 bp, Artificial Sequence
  Description of Artificial Sequence Synthetic construct
atggactggacctggatcctgttcctggtggccgccgccacccgcgtgcactccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctag

SEQ ID NO: 16 - DNA, 399 bp, Artificial Sequence
  Description of Artificial Sequence Synthetic construct
atggattggacctggatcctctttcttgtcgccgctgccactcgagtacattcaaactgggtaaatgtgatttccgaccttaaaaaaattgaagaccttatccaaagcatgcacatagacgccaccctttatactgaatccgacgtacacccctcctgcaaagttaccgccatgaaatgttttctcctcgaactccaagtaattagcctcgaatccggagacgcctctatccacgacacagttgaaaacctcataatccttgcaaataactctcttagctcaaacggaaatgttactgaatctggttgtaaagaatgcgaagaacttgaagaaaaaaatataaaagaatttctgcaatcatttgtccacatcgttcaaatgtttatcaatacctcttag

SEQ ID NO: 17 - DNA, 489 bp, Artificial Sequence
  Description of Artificial Sequence Synthetic construct
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtctggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 18 - DNA, 3737 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaccaccatggactggacctggatcctgttcctggtggccgccgccacccgcgtgcactccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctagtgagtcgacgggcgacgcgaaacttgggcccactcgagaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 19 - DNA, 3737 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaccaccatggattggacgtggatcctctttctcgtcgcggcggcgacgcgggtccattccaactgggtgaatgtcatttccgatctcaaaaaaattgaagatctcattcaatccatgcatattgatgcgacgctctatacggaatccgatgtccacccctcctgcaaagtcaccgcgatgaagtgctttctcctcgagctccaagtcatttccctcgagtccggggatgcgtccattcatgatacggtcgaaaatctgatcatcctcgcgaacaactccctctcctccaatgggaatgtcacggaatccgggtgcaaagaatgtgaggaactggaggaaaaaaatattaaagaatttctccagtcctttgtccatattgtccaaatgttcatcaacacgtcctagtgagtcgacgggcgacgcgaaacttgggcccactcgagaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt

SEQ ID NO: 20 - DNA, 3737 bp, Artificial Sequence
  Description of Artificial Sequence Synthetic construct
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgatatccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaccaccatggattggacctggatcctctttcttgtcgccgctgccactcgagtacattcaaactgggtaaatgtgatttccgaccttaaaaaaattgaagaccttatccaaagcatgcacatagacgccaccctttatactgaatccgacgtacacccctcctgcaaagttaccgccatgaaatgttttctcctcgaactccaagtaattagcctcgaatccggagacgcctctatccacgacacagttgaaaacctcataatccttgcaaataactctcttagctcaaacggaaatgttactgaatctggttgtaaagaatgcgaagaacttgaagaaaaaaatataaaagaatttctgcaatcatttgtccacatcgttcaaatgtttatcaatacctcttagtgagtcgacgggcgacgcgaaacttgggcccactcgagaggcgcgccgagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaattt